(19) United States
(12) Patent Application Publication
(10) Pub. No.: US 2024/0263190 A1
(43) Pub. Date: Aug. 8, 2024

(54) HUMAN IMMUNOGLOBULIN BINARY LIGHT CHAIN TRANSGENE CONSTRUCTS AND USES THEREOF

(71) Applicant: Gilead Sciences, Inc., Foster City, CA (US)

(72) Inventors: Daniel Rohrer, Los Gatos, CA (US); Peter Brams, Sacramento, CA (US)

(21) Appl. No.: 18/414,151

(22) Filed: Jan. 16, 2024

Related U.S. Application Data
(60) Provisional application No. 63/439,795, filed on Jan. 18, 2023.

(51) Int. Cl. C12N 15/85; C07K 16/18

(52) U.S. Cl. CPC C12N 15/8509; C07K 16/18; A01K 2207/15; A01K 2227/105

(57) ABSTRACT

Human immunoglobulin light chain transgene constructs are provided that encode two different rearranged light chain V-J regions arranged in such a manner that one of the two alternate light chains is expressed from the construct upon recombination in B cells. Transgenic animals comprising the transgene are also provided. The binary light chain transgene thus allows for expression of two alternate fixed light chains in the animals. Methods of using the transgenic animals are also provided.

### CROSS-REFERENCE TO RELATED APPLICATIONS

This application claims the benefit under 35 U.S.C. § 119(c) of U.S. Provisional Application No. 63/439,795, filed on Jan. 18, 2023, which is hereby incorporated herein by reference in its entirety for all purposes.

### SEQUENCE LISTING

The instant application contains a Sequence Listing which has been submitted electronically in .XML file format and is hereby incorporated by reference in its entirety. Said .XML copy, created on created on Dec. 13, 2023, is named ZL8016-WO-PCT_SL.xml and is 43,080 bytes in size.

### BACKGROUND OF THE DISCLOSURE

Bispecific antibodies (bsAb) combine two different antigen-binding sites in a single molecule. BsAbs can be advantageous over monospecific antibodies due to increased specificity in targeting and different mechanisms of action, potentially leading to higher clinical efficacies (reviewed in, e.g., Sedykh et al. (2018) Drug Design Devel. Ther. 12:195-208; Labrijn et al. (2019) Nat. Rev. Drug Discov. 18:585-608). The generation of bsAbs remains challenging, however, based on the necessity for correct pairing between two different heavy and light chains and related manufacturability issues. One approach that has been tried to reduce the complexities of bsAb generation and manufacturing is the use of a common light chain that combines with the two different heavy chains (e.g., as illustrated schematically in FIG. 1). This approach takes advantage of the well-established fact that for many antibodies the main driver for affinity and specificity is the heavy chain. Thus, use of a common light chain avoids the risk of improper heavy/light chain pairing and limits manufacturing to only three peptide chains while still retaining the desired antigen binding specificity for the bsAb.

Approaches to preparing common light chain transgenes and use of common light chains in bsAbs have been described (see e.g., Merchant et al. (1998) Nat. Biotech. 16:677-681; Jackman et al. (2010) J. Biol. Chem. 285:20850-20859; DeNardis et al. (2017) J. Biol. Chem. 292:14706-14717; Sharkey et al. (2017) MABS 9:257-268; Van Blarcom et al. (2018) MABS 10:256-268; PCT Publication No. WO 2011/097603; PCT Publication No. WO 2013/134263; PCT Publication No. WO 2015/153765; PCT Publication No. WO 2020/132557; PCT Publication No. WO 2020/205504).

While some advances have been made, additional approaches and compositions are needed for designing, preparing and using common light chain transgenes, in particular for use in bispecific antibodies.

### SUMMARY OF THE DISCLOSURE

The disclosure provides human immunoglobulin light chain transgene constructs that encode two different rearranged V-J regions positioned in opposite orientations in the construct, referred to herein as binary fixed light chain constructs. The constructs co-opt recombination signal sequences (RSSs) and RAG-mediated gene activation to silence the locus prior to recombination and to create functional expression of one V-J region post-recombination. More specifically, upon RAG-mediated recombination in B cells of an animal (e.g., mouse) carrying the transgene, one or other of the two alternate V-J regions is expressed. Thus, the binary fixed light chain transgene leads to a stochastic choice between two different pre-rearranged light chain V-J regions. The resultant immunoglobulin repertoire in the transgenic animal includes both fixed light chains, thereby providing two different common light chain options in the animal. This is advantageous in increasing the likelihood of successful antibody production to an antigen of interest in the animal.

Accordingly, in one aspect, the disclosure pertains to a transgene construct comprising:
(a) a first immunoglobulin light chain variable cassette (VL1) and a second immunoglobulin light chain variable cassette (VL2), wherein each of VL1 and VL2 comprise a promoter, a light chain V region, a light chain J region and a splice donor site;
(b) a stop cassette (SC) comprising a splice acceptor site and a polyadenylation signal; and
(c) a first recombination signal sequence (RSS) 12 mer (RSS1), a second recombination signal sequence 12 mer (RSS2) and an RSS 23mer (RSS3);
wherein the transgene construct comprises, from 5′ to 3′
VL1-RSS1-SC—RSS2-VL2-RSS3
and wherein VL2 is in antisense orientation relative to VL1.
   When the transgene construct is carried by a B cell, prior to RAG-mediated recombination VL1 and VL2 are inactive and after RAG-mediated recombination either VL1 or VL2 is active.

In embodiments, the light chain V regions and J regions are human kappa sequences. Non-limiting examples of suitable V and J regions are disclosed herein. In an embodiment, VL1 or VL2 comprises a Vk 1-39 region. In an embodiment, VL1 or VL2 comprises a Jk JK2 region. In an embodiment, VL1 or VL2 comprises a Vk 1-39 region and a Jk JK2 region. In an embodiment, VL1 or VL2 comprises a Vk 4-1 region. In an embodiment, VL1 or VL2 comprises a Jk JK4 region. In an embodiment, VL1 or VL2 comprises a Vk 4-1 region and a Jk JK4 region. In an embodiment, VL1 comprises a Vk 1-39 region and VL2 comprises a Vk 4-1 region. In an embodiment, VL1 comprises a Vk 1-39 region and a Jk JK2 region and VL2 comprises a Vk 4-1 region and Jk JK4 region. In an embodiment, VL1 comprises a Vk 4-1 region and VL2 comprises a Vk 1-39 region. In an embodiment, VL1 comprises a Vk 4-1 region and a Jk JK4 region and VL2 comprises a Vk 1-39 region and a Jk JK2 region.

In embodiments, the light chain V regions and J regions are human lambda sequences. Non-limiting examples of suitable lambda V and J regions are disclosed herein. In an embodiment, VL1 or VL2 comprises a VA 2-14 region. In an embodiment, VL1 or VL2 comprises a Jλ JL2 region. In an embodiment, VL1 or VL2 comprises a VA 2-14 region and a Jλ JL2 region. In an embodiment, VL1 or VL2 comprises a Vλ 1-40 region. In an embodiment, VL1 or VL2 comprises a Jλ JL1 region. In an embodiment, VL1 or VL2 comprises a VA 1-40 region and a Jλ JL1 region. In an embodiment, VL1 comprises a Vλ 2-14 region and VL2 comprises a Vλ 1-40 region. In an embodiment, VL1 comprises a VA 2-14 region and a Jλ JL2 region and VL2 comprises a Vλ 1-40 region and Jλ JL1 region. In an embodiment, VL1 comprises a VA 1-40 region and VL2 comprises a VA 2-14 region. In an embodiment, VL1 comprises a VA 1-40 region and a Jλ JL1 region and VL2 comprises a Vλ 2-14 region and a Jλ JL2 region.

In embodiments, the transgene construct further comprises a light chain constant region downstream of RSS3. In an embodiment, the light chain constant region is a human kappa constant region. In an embodiment, the transgene construct further comprises an enhancer downstream of RSS3 and upstream of the light chain constant region. In an embodiment, the enhancer comprises an intronic human kappa enhancer (hEKi).

In an embodiment, VL1 or VL2 of the transgene construct comprises a CDR3 comprising the sequence shown in SEQ ID NO: 1. In an embodiment, VL1 or VL2 of the transgene construct comprises a CDR3 comprising the sequence shown in SEQ ID NO: 2. In an embodiment, VL1 or VL2 of the transgene construct comprises the sequence shown in SEQ ID NO: 3. In an embodiment, VL1 or VL2 of the transgene construct comprises the sequence shown in SEQ ID NO: 4. In an embodiment, the transgene construct comprises the sequence shown in SEQ ID NO: 5.

In an embodiment, VL1 or VL2 of the transgene construct comprises a CDR3 comprising the sequence shown in SEQ ID NO: 6. In an embodiment, VL1 or VL2 of the transgene construct comprises a CDR3 comprising the sequence shown in SEQ ID NO: 7.

In another aspect, the disclosure pertains to a transgenic animal comprising a transgene construct of the disclosure. In an embodiment, the transgenic animal is a mouse. In an embodiment, the transgenic mouse further comprises a transgene construct encoding an immunoglobulin heavy chain such that the mouse expresses antibodies comprising the heavy chain paired with a light chain comprising the light chain V region of either the VL1 or VL2.

In another aspect, the disclosure pertains to a method of generating antibodies to an antigen of interest, the method comprising administering the antigen of interest to the transgenic animal (e.g., mouse) of the disclosure, such that antibodies that bind to the antigen of interest are generated. In an embodiment, the method further comprises isolating an antibody of interest from the animal and determining whether the antibody uses the light chain V region of VL1 or VL2.

### BRIEF DESCRIPTION OF THE DRAWINGS

FIG. 1 is a schematic diagram of a bispecific antibody approach that uses two distinct heavy chains and one fixed light chain to create two different binding specificities.

FIG. 2 is a schematic diagram of a binary light chain transgene construct, showing the inactive state prior to RAG activation and the alternate activation of VJK1 or VJK2 after RAG activation. PR>=promoter; SD=splice donor; SA=splice acceptor; pA=polyA/STOP cassette; hEKi=intronic human Kappa enhancer; SA/pA=cassette derived from the human IGLambda C2 locus and containing 2 consensus pA signals.

FIG. 3 is a schematic diagram illustrating how a binary fixed light chain model increases hit frequency as compared to a single fixed light chain model.

FIG. 4 is a schematic diagram illustrating construction elements of a representative binary fixed light chain construct of the disclosure.

FIG. 5 is a schematic diagram illustrating the structure of a representative binary fixed light chain construct of the disclosure.

FIG. 6. is a schematic diagram illustrating the insertion of a representative binary fixed light chain construct into the mouse kappa locus via recombination. LP=landing pad; FLC=fixed (AKA binary) light chain; neo^{R} and puro^{R} refer to neomycin and puromycin resistance cassettes, respectively.

FIG. 7 is a schematic diagram of a plasmid map of a representative transgene construct of the disclosure.

FIG. 8 shows the results of PCR amplicon and DNA sequencing analysis of recombined transgene alleles from transgenic mice.

FIG. 9 shows the results of RT-PCR analysis of spleen cell RNA from transgenic mice, showing expression of correctly-spliced transgene mRNA.

FIG. 10A-10C shows the results of ELISA assays for transgenic mice. FIG. 10A is a schematic illustration of the antibody format to be detected carrying the fixed human kappa LC encoded by the transgene. FIG. 10B shows the IgK levels in naïve (unimmunized mice). FIG. 10C shows the ELISA results for mice immunized with an COVID-19 spike protein antigen preparation.

FIG. 11 is a schematic diagram of a binary lambda fixed light chain (λ FLC) transgene construct, showing the inactive state of both light chain variable regions prior to RAG-mediated recombination and the activation of either λFLC1 or λFLC2 after RAG-mediated recombination.

### DETAILED DESCRIPTION

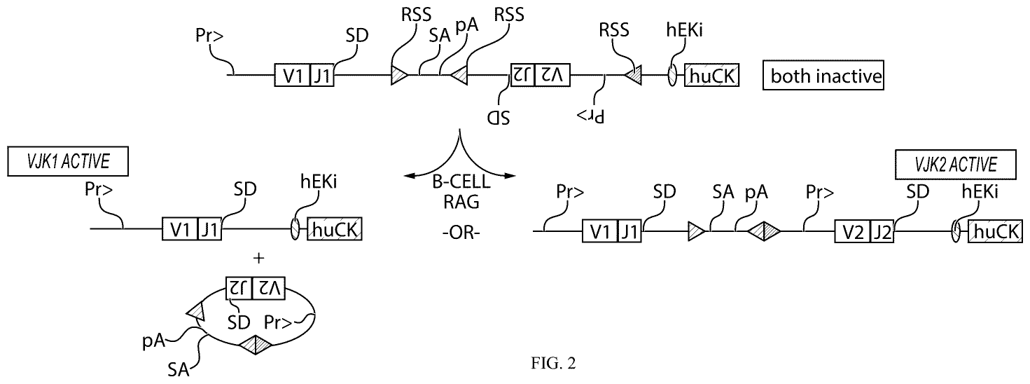

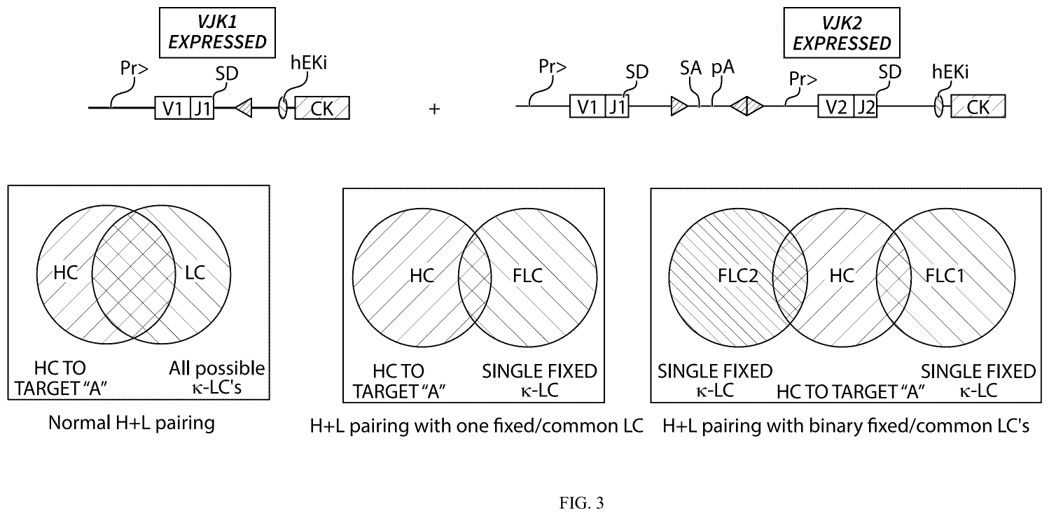

The binary fixed light chain transgene constructs of the disclosure encode two different V-J regions in opposite orientation, as illustrated schematically in FIG. 2. The constructs utilize the Recombination Signal Sequences (RSSs) and the endogenous Recombination Activation Gene (RAG) gene activation system to silence the expression of both V-J regions prior to recombination and to create functional expression of one or the other V-J region post-recombination. The design ensures that the pre-rearranged human light chains are inactive prior to B cell recombination, to help ensure that normal B cell development is not perturbed by having a transcriptionally active and functional light chain too early in development. Since typical B-cell differentiation proceeds by having the heavy chain rearrange prior to the light chain (see e.g., Yancopoulos and Alt (1986) Annu Rev Immunol. 4:339-68), all attempts were made to preserve this natural biology inherent to B-cell development. The overall intention behind this approach is thus to give the transgenic animal one of two alternate choices in terms of the human light chain that it will express, as illustrated schematically in FIG. 3. Thus, for antigens wherein a particular HC:LC pair is precluded or not antigen reactive, another choice is available, increasing the likelihood of obtaining an antibody of interest.

Various aspects of the disclosure are described in further detail below. Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer defined protocols and/or parameters unless otherwise noted.

### I. Construct Design

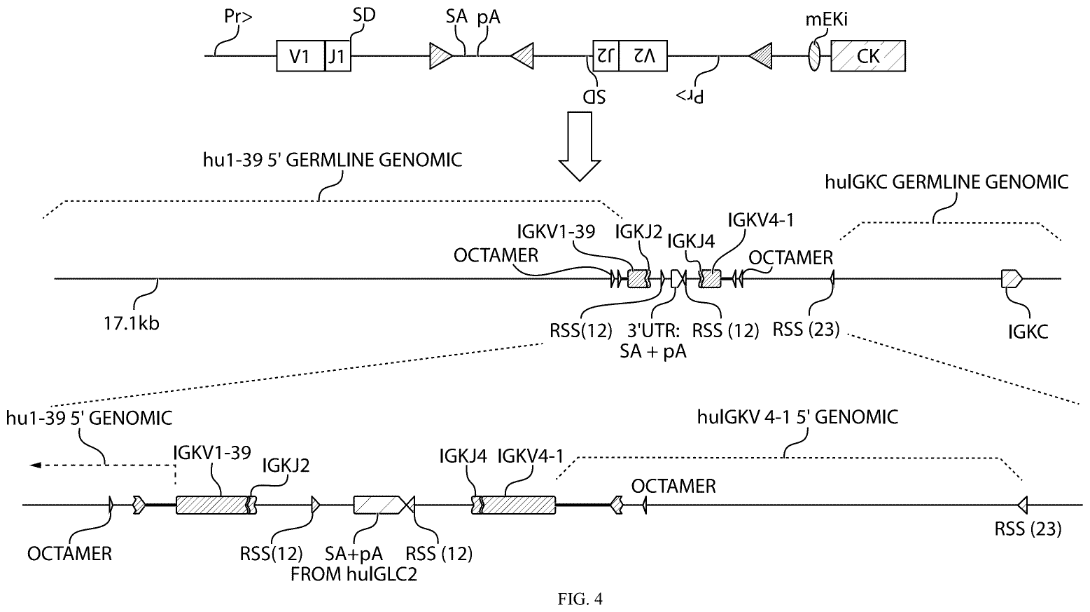

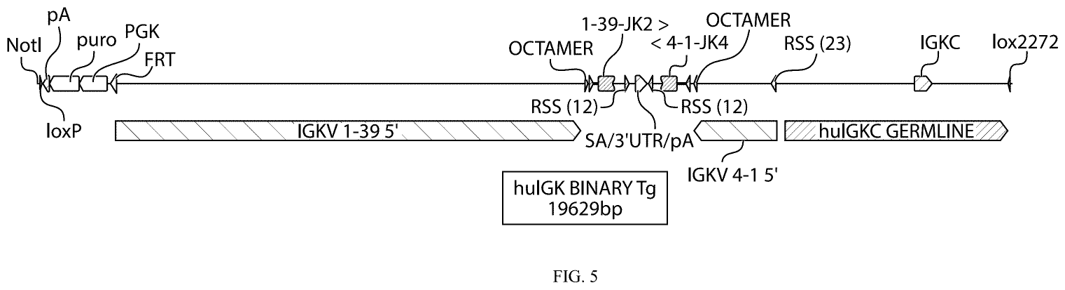

The design and construction of a binary fixed light chain transgene is described in detail in Example 1. As illustrated in FIG. 2, FIG. 4 and FIG. 5, the construct comprises two V-J region cassettes in opposite orientation (i.e., antisense) to each other, with a stop cassette between them. The construct also comprises intervening RSS sequences for facilitating recombination.

Thus, in an embodiment, the transgene construct comprising:
(a) a first immunoglobulin light chain variable cassette (VL1) and a second immunoglobulin light chain variable cassette (VL2), wherein each of VL1 and VL2 comprise a promoter, a light chain V region, a light chain J region and a splice donor site;
(b) a stop cassette (SC) comprising a splice acceptor site and a polyadenylation signal; and
(c) a first recombination signal sequence (RSS) 12 mer (RSS1), a second recombination signal sequence 12 mer (RSS2) and an RSS 23mer (RSS3);
wherein the transgene construct comprises, from 5′ to 3′
VL1-RSS1-SC—RSS2-VL2-RSS3
and wherein VL2 is in antisense (i.e., reversed or opposite) orientation relative to VL1.

With this structure of the transgene, neither VL cassette is capable of producing functional light chain transcripts prior to RAG-mediated recombination since the artificial splice/pA stop signal prevents the upstream VL1 from proper splicing and expression and the downstream VL2 is in the improper antisense orientation. Moreover, the RSSs are out of their normal context in that they are not joining coding sequences (the non-homologous end-joining that results occurs within the VL and kappa constant region intron and should not have an impact).

As further illustrated in FIG. 2, FIG. 4 and FIG. 5, following RAG-mediated recombination, either: (i) VL1 becomes active, through excision of the SA/pA stop cassette and the VL2 cassette and operative linkage of the VL1 promoter and coding sequences with the downstream enhancer and constant region sequences, or (ii) VL2 becomes active through inversion of the VL2 cassette, leading to operative linkage of the VL2 promoter and coding sequences with the downstream enhancer and constant region sequences.

Thus, when a transgene construct of the disclosure is carried by a B cell, prior to RAG-mediated recombination VL1 and VL2 are inactive and after RAG-mediated recombination either VL1 or VL2 is active.

In an embodiment, the light chain V and J regions used in the VL1 and VL2 cassettes are kappa regions (e.g., human kappa regions). A non-limiting examples of a human kappa light chain construct is described in detail in Example 1. In another embodiment, the light chain V and J regions used in the VL1 and VL2 cassettes are lambda regions (e.g., human lambda regions). A non-limiting example of a human lambda light chain construct is described in detail in Example 2. In another embodiment, one of the VL1/VL2 cassettes uses kappa regions and the other of VL1/VL2 cassette uses lambda regions. For example, a human kappa VL1 or VL2 cassette as described in Example 1 can be combined with a human lambda VL1 or VL2 cassette as described in Example 2 to create a binary fixed light chain construct comprising a kappa cassette and a lambda cassette.

Selection of the light chain V and J regions to be used in VL1 and VL2 can be based on one or more of several possible criteria. For example, if particular V/J regions are known to show propensity for binding to an antigen of interest, such regions may be selected for use with that intended antigen. Alternatively, for use with a wide range of antigens, V regions can be selected based on: (i) the frequency of expression of in the normal human population; (ii) the ability to pair with a diversity of human heavy chain families, if known; and/or (iii) the ability to pair with known J domains. Information on V region usage frequency and preferred VK/VH pairings is available in the art and can be used in the design of the construct. For example, VK regions may be selected based on their preferred pairings with VH regions. Non-limiting examples of such preferred pairings are described in DeKosky et al. (2015) Nat. Med. 21:86-91, the entire contents of which are expressly incorporated herein by reference.

In an embodiment, the V regions used in VL1 and VL2 are human Vk regions independently selected from the group consisting of Vk regions 1-5, 1-6, 1-8, 1D-8, 1-9, 1-12, 1D-12, 1-13, 1D-13, 1-16, 1D-16, 1-17, 1D-17, 1-27, 1-33, 1D-33, 1-37, 1D-37, 1-39, 1D-39, 1D-42, 1D-43, 1-NL1, 2-24, 2-28, 2D-28, 2-29, 2D-29, 2-30, 2D-30, 2-40, 2D-40, 3-7, 3D-7, 3-11, 3D-11, 3-15, 3D-15, 3-20, 3D-20, 4-1, 5-2, 6-21, 6D-21 and 6D-41.

In an embodiment, the V regions used in VL1 and VL2 are human Vk regions independently selected from the group consisting of Vk regions 1-5, 1-39, 3-11, 3-15, 3-20, 3-28 and 4-1.

In an embodiment, the V regions used in VL1 and VL2 are human Vk regions independently selected from the group consisting of Vk regions 1-39, 3-20 and 4-1. In an embodiment, VL1 or VL2 comprises a Vk 1-39 region. In an embodiment, VL1 or VL2 comprises a Vk 4-1 region. In an embodiment, VL1 comprises a Vk 1-39 region and VL2 comprises a Vk 4-1 region. In an embodiment, VL1 comprises a Vk 4-1 region and VL2 comprises a Vk 1-39 region.

In an embodiment, the J regions used in VL1 and VL2 are human Jk regions (e.g., when Vk regions are used). In an embodiment, the J regions used in VL1 and VL2 are human Jλ regions (e.g., when Vλ regions are used). In an embodiment, the J regions used are human Jk regions selected from the group consisting of Jk JK1, JK2, JK3, JK4 and JK5. Information available in the art on commonly observed IGKV-IGKJ pairings can be used in the design of the VL1 and VL2 regions. For example, Jk regions may be selected based on their preferred pairings with Vk regions. Non-limiting examples of such preferred pairings are described in Collins et al. (2008) Immunogenetics 60:669-676, the entire contents of which are expressly incorporated herein by reference. In an embodiment, VL1 or VL2 comprises a Jk JK2 region. In an embodiment, VL1 or VL2 comprises a Vk 1-39 region and a Jk JK2 region. In an embodiment, VL1 or VL2 comprises a Jk JK4 region. In an embodiment, VL1 or VL2 comprises a Vk 4-1 region and a Jk JK4 region. In an embodiment, VL1 comprises a Vk 1-39 region and a Jk JK2 region and VL2 comprises a Vk 4-1 region and Jk JK4 region. In an embodiment, VL1 comprises a Vk 4-1 region and a Jk JK4 region and VL2 comprises a Vk 1-39 region and a Jk JK2 region.

In an embodiment, the V regions used in VL1 and VL2 are human Vλ regions, for example independently selected from the group consisting of Vλ regions 2-14, 3-19, 3-21, 3-1, 1-51 and 1-40.

In an embodiment, the V regions used in VL1 and VL2 are human Vλ regions independently selected from the group consisting of Vλ regions 2-14, 3-19 and 1-40. In an embodiment, VL1 or VL2 comprises a Vλ 2-14 region. In an embodiment, VL1 or VL2 comprises a Vλ 1-40 region. In an embodiment, VL1 comprises a Vλ 2-14 region and VL2 comprises a Vλ 1-40 region. In an embodiment, VL1 comprises a Vλ 1-40 region and VL2 comprises a Vλ 2-14 region.

In an embodiment, the J regions used in VL1 and VL2 are human Jλ regions (e.g., when Vλ regions are used). In an embodiment, the J region used is human Jλ JL1 or JL2. In an embodiment, VL1 or VL2 comprises a Jλ JL2 region. In an embodiment, VL1 or VL2 comprises a Vλ 2-14 region and a Jλ JL2 region. In an embodiment, VL1 or VL2 comprises a Jλ JL1 region. In an embodiment, VL1 or VL2 comprises a Vλ 1-40 region and a Jλ JL1 region. In an embodiment, VL1 comprises a Vλ 2-14 region and a Jλ JL2 region and VL2 comprises a Vλ 1-40 region and Jλ JL1 region. In an embodiment, VL1 comprises a Vλ 1-40 region and a Jλ JL1 region and VL2 comprises a Vλ 2-14 region and a Jλ JL2 region.

Each VL1 and VL2 cassette also contains a promoter operatively linked to the V-J coding sequences so as to drive expression of the V-J coding region upon activation of the cassette. Suitable promoters are well-established in the art, including endogenous mouse or human immunoglobulin promoters, as well as heterologous promoters. In an embodiment, the VL1 and VL2 cassettes use the endogenous promoter of the V region incorporated into the cassette.

Each VL1 and VL2 cassette also comprises an operatively linked splice donor site, whereas the intervening stop cassette (SC) comprises an operatively linked splice acceptor site. Standard splice donor and acceptor site sequences, well established in the art, are used. The SC also comprises a polyadenylation signal(s), standard sequences for which are used and are well established in the art. In an embodiment, a stop cassette is derived from the human Ig lambda C2 locus and contains two consensus polyadenylation signals.

Recombination Signal Sequences (RSS) are included in the construct to facilitate RAG-mediated recombination. Two operatively linked RSS 12mer sequences (RSS1 and RSS2) are positioned upstream and downstream, respectively, of the SC (in opposite orientations). An operatively linked RSS 23mer sequence (RSS3) is positioned downstream of VL2. Standard RSS 12mer and 23mer sequences, well established in the art, are used. In an embodiment, the two RSS 12mer sequences are derived from the human VK 1-39*01 allele and are used in two opposite orientations. In an embodiment, the RSS 23mer is from the human IGKJ1*01 allele.

In embodiments, the transgene construct further comprises a light chain constant region downstream of RSS3. In an embodiment, the light chain constant region is a human Ig kappa constant region. In an embodiment, the light chain constant region is a human Ig lambda constant region. In an embodiment, the light chain constant region is a mouse kappa constant region. In an embodiment, the light chain constant region is a mouse lambda constant region.

In an embodiment, the transgene construct further comprises an enhancer downstream of RSS3 and upstream of the light chain constant region. Suitable enhancers are well-established in the art, including endogenous mouse or human immunoglobulin enhancers, as well as heterologous enhancers. In an embodiment, the enhancer comprises an intronic human kappa enhancer (hEKi).

The transgene construct can further include immunoglobulin locus genomic sequences upstream (5′) of the VL1 and VL2 cassettes to act as a genetic “buffer” of sorts and to include minor or subtle regulatory elements from the Ig locus. For example, in an embodiment, the VL1 cassette uses a VK 1-39 variable region and approximately 9.7 kb of 5′ genomic sequence from VK 1-39 is incorporated upstream of the VL1 cassette. Similarly, in an embodiment, the VL2 cassette uses a VK 4-1 variable region and approximately 1.6 kb of 5′ genomic sequence from VK 4-1 is incorporated upstream of the VL2 cassette.

The transgene construct can further include immunoglobulin locus genomic sequences upstream (5′) of constant region coding sequences. For example, in an embodiment, the construct incorporates a human Ig kappa constant region and includes approximately 2.8 kb of genomic DNA 5′ to the Ck region coding sequences, which region stays intact regardless of whether the VL1 or VL2 cassette gets functionally activated.

The nucleotide sequence of the transgene construct can be further optimized for intended purposes. For example, the construct can be altered for codon optimization (e.g., to increase expression of the encoded regions). Approaches for codon optimization are well established in the art.

The transgene construct can further comprise sequences that allow for targeted insertion of the transgene into a specific locus, e.g., an endogenous mouse light chain locus. Knock-in technology for replacing an endogenous locus with a targeted transgene is well established in the art. In a preferred embodiment, the transgene construct comprises recombination sequences allowing for the transgene to be knocked-in to the endogenous mouse kappa locus, thereby deleting all of the endogenous mouse Vk, Jk and Ck sequences. In an embodiment, the construct comprises an upstream loxP site and a downstream lox2272 site. Insertion of the transgene into the genome of a host via recombination is described further in Section III.

In an embodiment, VL1 or VL2 encodes a commonly observed CDR3 sequence of Vk1-39/JK2, the amino acid sequence of which CDR3 is shown in SEQ ID NO: 1. In an embodiment, VL1 or VL2 encodes a commonly observed CDR3 sequence of Vk4-1/JK4, the amino acid sequence of which CDR3 is shown in SEQ ID NO: 2. In an embodiment, VL1 or VL2 encodes the Vk1-39/JK2 amino acid sequence shown in SEQ ID NO: 3. In an embodiment, VL1 or VL2 encodes the Vk4-1/JK4 amino acid sequence shown in SEQ ID NO: 4.

In an embodiment, the binary fixed light chain construct comprises the nucleotide sequence shown in SEQ ID NO: 5.

In an embodiment, VL1 or VL2 encodes a CDR3 as shown in SEQ ID NO: 6, which represents a consensus Vλ 2-14/JL2 CDR3 sequence. In an embodiment, VL1 or VL2 encodes a CDR3 as shown in SEQ ID NO: 7, which represents a consensus Vλ 1-40/JL1 CDR3 sequence.

### II. Construct Preparation

The transgene constructs of the disclosure can be prepared using standard recombinant DNA techniques. Cloning vectors containing polylinkers are useful as starting vectors for insertion of DNA fragments of interest. Suitable cloning vectors are well established in the art. Moreover, plasmids or other vectors (e.g., YACs) carrying human unrearranged light chain immunoglobulin sequences have been described in the art (see e.g., U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; and U.S. Pat. Nos. 5,939,598; 6,075,181; 6,114,598; 6,150,584 and 6,162,963, all to Kucherlapati et al.) and can be used as a source of V and J region sequences. Alternatively, desired sequences can be synthesized by standard methods. The appropriate DNA fragments are then operatively linked through ligation into a cloning vector, followed by characterization of the vector (e.g., by restriction fragment analysis or sequencing or the like) to ensure proper arrangement of the fragments.

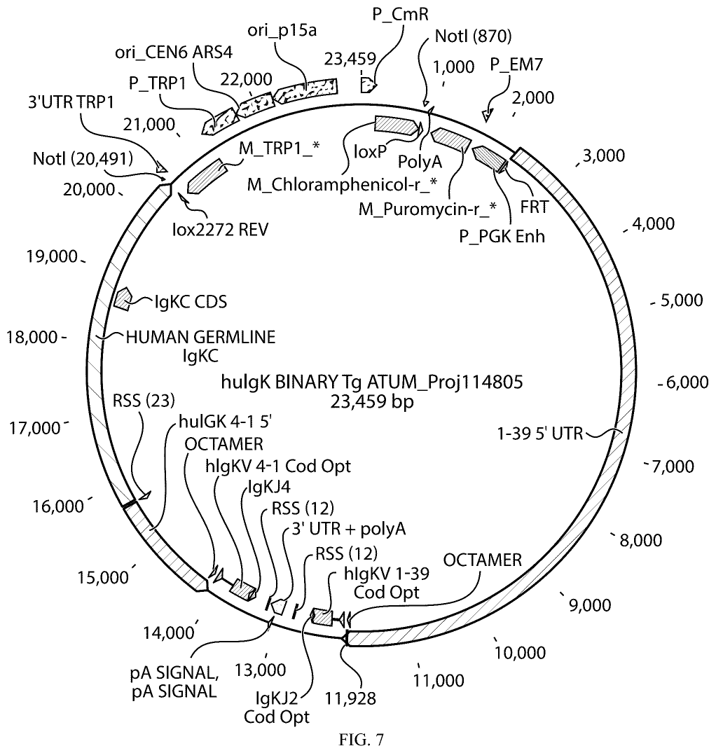

A non-limiting example of a binary fixed light chain vector of the disclosure is illustrated schematically in FIG. 7.

To prepare the transgene construct for microinjection or other technique for transgenesis, the transgene construct can be isolated from the vector in which it is carried by cleavage with appropriate restriction enzymes to release the transgene construct fragment. The fragment can be isolated using standard techniques, such as by pulse field gel electrophoresis on an agarose gel, followed by isolation of the fragment from the agarose gel, such as by [beta]-agarase digestion or by electroelution. For example, the agarose gel slice containing the transgene construct fragment can be excised from the gel and the agarose can be digested with [beta]-agarase (e.g., from Takara), using standard methodology. Alternatively, the transgene can be prepared as intact, supercoiled plasmid, making use of sequence-specific recombinases to effect recombination of the transgene insert with compatible recombinase sites already present in the mouse Ig kappa locus.

### III. Preparation of Transgenic Animals

Another aspect of the disclosure pertains to a transgenic non-human host animal that comprises a transgene construct of the disclosure (i.e., the transgene construct is integrated into the genome of the host animal), such that the animal expresses an immune repertoire that comprises antibodies using fixed light chains comprising the V-J region of the VL1 cassette or the V-J region of the VL2 cassette. The transgenic non-human host animals of the disclosure are prepared using standard methods known in the art for introducing exogenous nucleic acid into the genome of a non-human animal or cells of a non-human animal (e.g., embryonic stem cells). In a preferred embodiment, the transgene construct is inserted into the genome of the host animal or host cell using knock-in technology to replace all or a part of an endogenous light chain locus (e.g., an endogenous kappa chain locus) with the transgene (e.g., a kappa chain transgene).

For knock-in approaches, typically loxP flanking sites are included in the construct such that the sites enable site-specific recombination between the donor transgene and the host genome, which has been modified with similar loxP sites to facilitate site- and orientation-specific recombination upon expression of Cre recombinase (so-called Recombinase Mediated Cassette Exchange, or RMCE). Recombination is performed in embryonic stem cells (e.g., mouse embryonic stem cells) and then embryonic stem cells with the modification of interest are implanted into a viable blastocyst, which then grows into a mature chimeric animal (e.g., mouse) with some cells having the original blastocyst cell genetic information and other cells having the modifications introduced to the embryonic stem cells. Subsequent offspring of the chimeric animal will then have the gene knock-in. Knock-in technology is summarized in, for example, Manis (2007) New Engl. J. Med. 357:2426-2429 and Turan et al. (2011) J. Mol. Biol. 407:193-221.

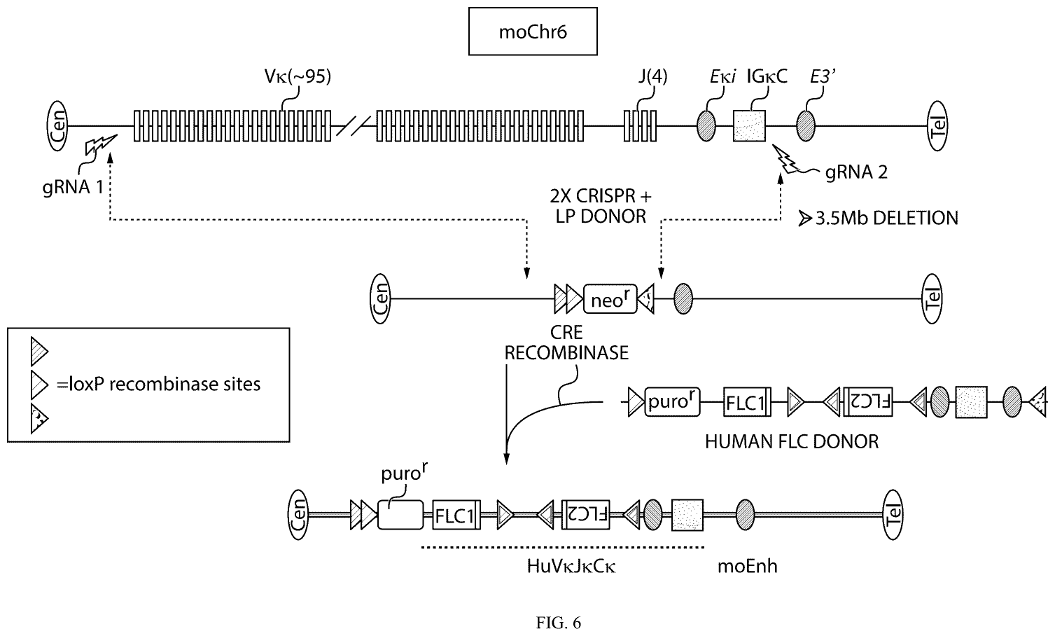

In a preferred embodiment, the transgene construct is inserted into the genome of a mouse, whose endogenous light chain locus has been modified to delete all Vk. Jk and Ck sequences, while simultaneously introducing loxP sites that are compatible with the donor transgene. In a preferred embodiment, the transgene construct is a kappa light chain construct that is inserted into the modified endogenous mouse kappa locus by homologous recombination, wherein all of the mouse Vk. Jk and Ck sequences have been deleted. In a preferred embodiment, the endogenous kappa locus is first engineered to delete VK through CK (˜3.5 million bp deletion) while leaving behind a region containing compatible loxP and lox2272 sites, referred to herein as a “landing pad” site. Subsequently the binary LC transgene construct is introduced into the landing pad site in ES cells with cre recombinase (referred to as Recombinase Mediate Cassette Exchange, or RMCE), as illustrated schematically in FIG. 6.

In another embodiment, a kappa light chain transgene that lacks a constant region is inserted into the endogenous kappa locus such that the Vk and Jk sequences are deleted but the Ck sequences are left intact and operatively linked to the functional variable region of the transgene, thereby producing chimeric antibodies in the mice (which can be reverse engineered to be fully human).

A standard method for preparing a transgenic non human animal, in particular a transgenic mouse, is to make a genetic modification, such as a knockin, in embryonic stem (ES) cells. Such modified stem cells can then be used to create chimeric mice via microinjection of pre-implantation stage mouse embryos, and the chimeras bred in turn to transmit the gene of interest or knockin allele. At this point, the knockin allele can be inerbred or cross-bred for line expansion and further study. Southern blot analysis, PCR or other such technique for analyzing genomic DNA is used to detect the presence of a unique nucleic acid fragment that would not be present in the non-transgenic animal or ES cell but would be present in the transgenic animal or ES cell. Selective breeding of transgenic offspring allows for homozygosity of the transgene to be achieved.

Although the preferred embodiment of the disclosure comprises transgenic mice, the invention encompasses other non-human host animals, including but not limited to rats, rabbits, pigs, goats, sheep, cows and chickens. Techniques for creating transgenic animals of each of these species have been described in the art. For example, preparation of transgenic rats is described in Tesson, L. et al. (2005) Transgenic Res. 14:531-546, including by techniques such as DNA microinjection, lentiviral vector mediated DNA transfer into early embryos and sperm-mediated transgenesis. Methods of transgenesis in rats are also described in Mullin, L. J. et al. (2002) Methods Mol. Biol. 180:255-270. Preparation of transgenic rabbits is described in, for example, Fan, J. et al. (1999) Pathol. Int. 49:583-594; Fan, J. and Watanabe, T. (2000) J. Atheroscler. Thromb. 7:26-32; Bosze, Z. et al (2003) Transgenic Res. 12:541-553. Preparation of transgenic pigs is described in, for example, Zhou, C Y. et al. (2002) Xenotransplantation 9:183-190; Vodicka, P. et al. (2005) Ann. N.Y. Acad. Sci. 1049:161-171.

Alternative transgenesis techniques to pronuclear microinjection in pigs include adenovirus mediated introduction of DNA into pig sperm (see e.g., Farre, L. et al. (1999) Mol. Reprod. Dev. 53:149-158) and linker-based sperm-mediated gene transfer (Chang, K. et al. (2002) BMC Biotechnol. 2:5). Preparation of transgenic goats is described in, for example, Ebert. K. M. et al. (1991) Biotechnology (NY) 9:835-838; Baldassarre, H. et al. (2004) Reprod. Fertil. Dev. 16:465-470. Somatic cell nuclear transfer in goats is described in, for example, Behboodi, E. et al. (2004) Transgenic Res. 11:215-224. Preparation of transgenic sheep is described in, for example, Ward, K. A. and Brown, B. W. (1998) Reprod. Fertil. Dev. 10:659-665. Preparation of transgenic cows is described in, for example, Donovan, D. M. et al. (2005) Transgenic Res. 14:563-567. Gene transfection of donor cells for nuclear transfer of bovine embryos is described in, for example, Lee S. L. et al. (2005) Mol. Reprod. Dev. 72:191-200. Preparation of transgenic domestic farm animals is also reviewed in Niemann, H. et al. (2005) Rev. Sci. Tech. 24:285-298. Preparation of transgenic chickens is described in, for example, Pain, B. et al. (1999) Cells Tissues Organs 165:212-219; Lillico, S. G. et al. (2005) Drug Discov. Today 10:191-196; and Ishii, Y. et al. (2004) Dev. Dyn. 229:630-642.

An animal of the disclosure (e.g., mouse) carrying a binary light chain transgene construct can be cross-bred with an animal (e.g., mouse) that carries an immunoglobulin heavy chain transgene to thereby produce an animal (e.g., mouse) that expresses antibodies comprising the heavy chain paired with a light chain comprising the light chain V region of either VL1 or VL2. Immunoglobulin heavy chain transgenic animals (e.g., mice) are well-established in the art.

In one embodiment, a transgenic animal (e.g., mouse) of the disclosure is heterozygous for the binary light chain construct and the other endogenous mouse light chain allele is inactivated such that either VL1 or VL2 is expressed in all light chains of transgenic cells after recombination of the transgene. In another embodiment, a transgenic animal (e.g., mouse) of the disclosure is homozygous for the binary light chain construct, in which case both alleles of the binary LC construct may be expressed in the animal (e.g., mouse) such that both VL1 and VL2 are utilized in the light chain repertoire.

### IV. Use of Transgenic Animals

The transgenic animals of the disclosure are useful for generating antibodies to a wide variety of antigens of interest. For animals carrying only the binary light chain transgene and an endogenous heavy chain locus, the animal will produce chimeric light chain/heavy chain antibodies that, if desired, can be reverse engineered to pair the light chain with a heavy chain of the same species. Alternatively, for animals (e.g., mice) carrying both a binary light chain Ig transgene (e.g., human) and a heavy chain Ig transgene (e.g., human, or human-mouse chimerics), partial or fully heterologous antibodies (e.g., fully human antibodies) can be prepared in the host transgenic animal.

Accordingly, in another aspect, the disclosure pertains to a method of generating antibodies to an antigen of interest, the method comprising administering the antigen of interest to a transgenic animal of the disclosure. In an embodiment, the animal is a transgenic mouse and the antigen is administered to the mouse such that antibodies that bind to the antigen of interest are generated in the mouse. In an embodiment, the animal is a transgenic mouse carrying both a human Ig binary light chain transgene and a human Ig heavy chain transgene and the antigen is administered to the mouse such that human antibodies that bind to the antigen of interest are generated in the mouse. In an embodiment, the method can further comprise isolating an antibody of interest from the host animal (e.g., mouse) and determining whether the antibody uses the light chain V region of VL1 or VL2.

Transgenic animals can be immunized with an antigen(s) of interest by standard methodologies known in the art and antibodies generated in the animals can be isolated and characterized also by standard established methods. Polyclonal antibodies can be directly isolated form the host animal and monoclonal antibodies can be prepared by standard methods, such as hybridoma technology or single B-cell cloning. Procedures for making monoclonal antibodies using hybridomas are well established in the art (see, e.g., U.S. Pat. No. 4,977,081, PCT Publication WO 97/16537, and European Patent No. 491057BJ, the disclosures of which are incorporated herein by reference). Alternatively, in vitro production of monoclonal antibodies from cloned cDNA molecules is also established in the art (see e.g., Andris-Widhopf et al. (2000) J. Immunol. Methods 242:159; and Burton (1995) Immunotechnology 1:87, the disclosures of which are incorporated herein by reference). Technologies to aid in the identification or retrieval of single B cells for cloning or cDNA capture are well established (for review, see e.g., Pedrioli and Oxenius (2021) Trends Immunol. 42:1143-1158). B cell clones from the immunized transgenic animals can be isolated and cDNAs encoding the antibodies can be isolated and cloned by standard molecular biology techniques into expression vectors. Further recombinant engineering of the cloned Ig cDNAs is also possible and well established in the art.

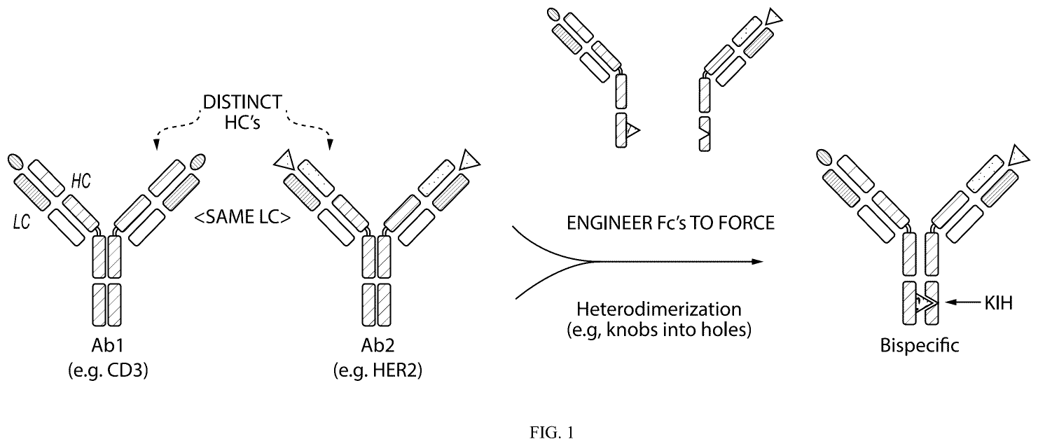

In an embodiment, a fixed light chain encoded by a transgene construct of the disclosure is identified as binding to a target of interest and then the fixed light chain is incorporated into a bispecific antibody (bsAb) that binds the target of interest. An exemplary bsAb approach that incorporates use of a fixed light chain with two different heavy chains is illustrated schematically in FIG. 1.

### V. Definitions

As used herein. “common light-chain” or “common immunoglobulin light-chain” or “single light chain” refers to a light chain variable region that can pair with multiple heavy chain variable regions to produce antibodies that bind to different antigens. For example, both arms of a bi-specific antibody can utilize the same light-chain (i.e., a “common” light chain) and different heavy chains (which largely determine the binding specificity of the arm).

As used herein, the term “operatively linked” is intended to describe the configuration of a nucleic acid sequence that is placed into a functional relationship with another nucleic acid sequence. For example, a promoter or enhancer is operatively linked to a coding sequence if it affects the transcription of the sequence. With respect to the joining of two protein coding regions, operatively linked means that the nucleic acid sequences being linked are contiguous and in reading frame. For splice donor/acceptor and RSS sequences, operatively linked means that the sequences are capable of effecting their functional purposes.

As used herein, “promoter” refers to a nucleic acid sequence which is required for expression of a gene product operably linked to the promoter/regulator sequence. In some embodiments, this sequence may be the core promoter sequence. In some embodiments, this sequence may also include an enhancer sequence and other regulatory element(s) required for expression of the gene product.

As used herein, the term “rearranged” in reference to an immunoglobulin V segment refers to the configuration wherein the V segment is positioned immediately adjacent to a J segment so as to encode essentially a complete VL domain. A rearranged variable region locus can be identified by comparison to germline DNA.

As used herein, the term “transgene” refers to a gene that is introduced as an exogenous source to a site within a host genome (e.g., mouse light chain Ig locus).

As used herein, the term “transgene construct” refers to a nucleic acid preparation suitable for introduction into the genome of a host animal.

As used herein, the term “transgenic mouse” refers to a mouse comprising cells having a transgene, as defined herein. The transgene may be present in all or some cells of the mouse.

The present invention is further illustrated by the following examples, which should not be construed as further limiting. The contents of figures and all references, patents and published patent applications cited throughout this application are expressly incorporated herein by reference.

### EXAMPLES

### Example 1: Preparation of Human Kappa Binary Common Light Chain Constructs

In this example, the preparation of transgene constructs for expressing one of two alternate “fixed” human kappa light chains is described. The fixed nature of the light chains means that the VK and J segments to be expressed are already recombined in a manner that precludes the normal and stochastic VK-J recombination that naturally occurs in early B cell development.

### Conceptual Framework

The intention behind the binary common light chain concept is to give the transgenic mouse one of two alternate choices in terms of the human kappa LC that it will express. By doing so, this helps enable a Common Light Chain (CLC) approach that can be used for a bispecific antibody (bsAb) platform. One of the more successful bsAb approaches is to make use of a CLC that is shared between two unrelated heavy chains that together with their CLC, encode two different antigen-specificities while making use of the same LC, as illustrated schematically in FIG. 1. In this bispecific antibody format, Ab1 and Ab2 come from transgenic mice with normal and unrearranged human or mouse VH repertoires, but with a fixed light chain that is known to be HC promiscuous. This format is known to exhibit low immunogenicity, expression levels are similar to regular antibodies, and the bispecific antibody product possesses normal Fc-receptor interactions. Moreover, this format does not suffer from some of the other more complicated bispecific antibody schemes in terms of HC:LC pairing combinations and the engineering required to force proper pairing or selectively purify the properly paired species (for a review of such schemes, see e.g., Kontermann and Brinkmann (2015) Drug Discovery Today 20(7).

An advantageous feature of the binary common light chain construct described herein is that two different CLC's are provided for the mouse to ‘choose’ from; so in those instances where a particular HC:LC pair is precluded or non-antigen reactive, another choice is available. The selection of kappa light chains from which to choose the fixed or common allele typically is based on: (i) the frequency of expression in normal human populations; (ii) the ability to pair with a diversity of human HC families (where known); and (iii) the ability to pair with known J domains.

### Binary Light Chain Construct Design

A representative schematic diagram of the binary light chain transgene construct is shown in FIG. 2. The binary transgene co-opts the Recombination Signal Sequences (RSSs) and Recombination Activation Gene (RAG) gene activation system to present with a silenced locus prior to recombination and to create functional expression post-recombination, leading to a stochastic choice between two different pre-rearranged kappa light chains. Neither cassette is active prior to RAG action; the artificial splice/pA stop signal prevents upstream and properly oriented sequences from functional light chain expression. The RSSs are out of their normal context in that they are not joining coding sequences; rather, they reside within non-coding introns so as not to interfere with coding sequences or RNA splicing behavior (non-homologous end-joining in this situation should thus not have an impact).

Following RAG-mediated recombination, either: (i) V1-J1 becomes active, through excision of the SA/pA stop cassette and the V2-J2 cassette, and operative linkage of the V1-J1 promoter and coding sequences with the downstream enhancer and constant region sequences, or (ii) V2-J2 becomes active through inversion of the V2-J2 cassette, leading to operative linkage of the V2-J2 promoter and coding sequences with the downstream enhancer and constant region sequences.

As illustrated schematically in FIG. 3, the use of a binary fixed light chain approach, giving the ability to express two possible light chains, should lead to increased hit frequency since having two light chain choices increases the odds of a successful pairing with any given heavy chain, providing more binding solutions to the two antigens of interest. Ultimately, the mouse ‘decides’ the best light chain fit.

### Light Chain V Region Selection

The frequency of expression of VK regions in human populations was used as a starting point for light chain V region selection. The IMGTO database provided information on VK frequency, leading to the determination that VK 1-39 and 3-20 were the most frequently used, although 4-1, 3-11, 3-15, 3-28 and 1-5 were also observed at high frequency. VH/VK pairings were also analyzed based on data disclosed in DeKosky et al. (2015) Nat. Med. 21:86-91. This analysis revealed that the combination of VK 1-39 and 4-1 should allow for diverse VH pairings across most VH families.

Further BLAST analysis was performed to identify a commonly used LCDR3 from VK 1-39+JK2 and from VK 4-1+JK4. For VK 1-39+JK2, a common LCDR3 was identified as having the sequence CQQSYSTPYTF (SEQ ID NO: 1). For VK 4-1+JK4, a common LCDR3 was identified as having the sequence CQQYYSTPLTF (SEQ ID NO: 2). Optimized alleles using these most common LCDR3s were then prepared for VK 1-39+JK2 and VK 4-1+JK4, having the amino acid sequences shown in SEQ ID NOs: 3 and 4, respectively.

Further design of the light chain sequence can include alteration of somatic hypermutation (SHM) sites and codon optimization for expression. When balancing considerations for sequence modifications, optimizing for good expression was chosen over alteration of SHM sites.

### Transgene Construction and Mouse Preparation

Construction of the binary fixed light chain transgene construct is illustrated schematically in FIG. 4. The RSS (12) elements are derived from the huVK 1-39*01 allele (in 2 opposite orientations). The RSS (23) element is from the huIGKJ1*01 allele. The cassette which incorporates the splice acceptor (SA) and polyadenylation (pA) sequences is derived from the human IGLambda C2 locus and contains two consensus pA signals. Approximately 9.7 kb of genomic 5′ sequence from VK 1-39 is included at the 5′ end to provide both a genetic “buffer” of sorts, but also to include potential minor or subtle regulatory elements from this region. The VK 4-1 element has a smaller upstream regulatory element (1.6 kb), but owing to the mechanism of binary switching, this should always be “protected” by the 1-39 5′ segment. Additionally, there is approximately 2.8 kb of information 5′ to the human IGKC coding region included in this construct, which stays intact regardless of which VK segment gets functionally recombined. The start of this region is defined by the location of the RSS (23) site. Still further, there is approximately 560 bp of information 3′ of the human IGKC segment, meant to align with coordinates of the CRISPR-edited mouse genomic IGKC locus landing pad.

The binary fixed light chain transgene is further illustrated schematically in FIG. 5, which shows how the direction of the 1-39 cassette is opposite that of the 4-1 cassette (i.e., the direction of the 4-1 cassette is antisense to that of the 1-39 cassette). The Splice Acceptor/3′UTR/polyA downstream of 1-39 should prevent any splicing or translation of a functional kappa light chain that includes the kappa C exon. Moreover, the 4-1 cassette, being in antisense orientation, is also blocked for functional splicing. The action of RAG proteins on the construct during B cell development effects RSS recombination and stochastically activates either the 1-39 or the 4-1 cassette. The nucleotide sequence of a representative binary fixed light chain transgene (as illustrated in FIG. 5) is shown in SEQ ID NO: 5.

The binary fixed light chain transgene construct was knocked into a mouse kappa locus (previously deleted of all mouse Vk, Jk and kC information) using standard technology known in the art via cre/lox mediated RMCE (Recombinase Mediated Cassette Exchange), as illustrated schematically in FIG. 6. The resultant mouse thus expressed the end product of either a fully human 1-39/J2/kC light chain or a 4-1/J4/kC light chain. Twenty-three ES clones were successfully knocked-in and four were sequence-validated by Targeted Locus Amplification (TLA) technology. Mice containing the binary fixed light chain transgene can be bred to homozygosity. Mice can be cross-bred with a full-diversity heavy chain mouse (e.g., a humanized or human HC transgenic mouse).

The final plasmid-based transgene construct, including appropriate vector sequences, is illustrated schematically in FIG. 7.

Functional validation of the binary fixed light chain mice can include analysis of genomic DNA to show that recombination occurs in B cells to generate the two different light chain alleles, RNA analysis to show in-frame, spliced transcripts can be generated in mouse B cells and examination of protein levels of the kappa light chains. The titer and immune responsiveness to test antigens can also be examined and compared to wild type mice.

### Transgenic Mice Characterization

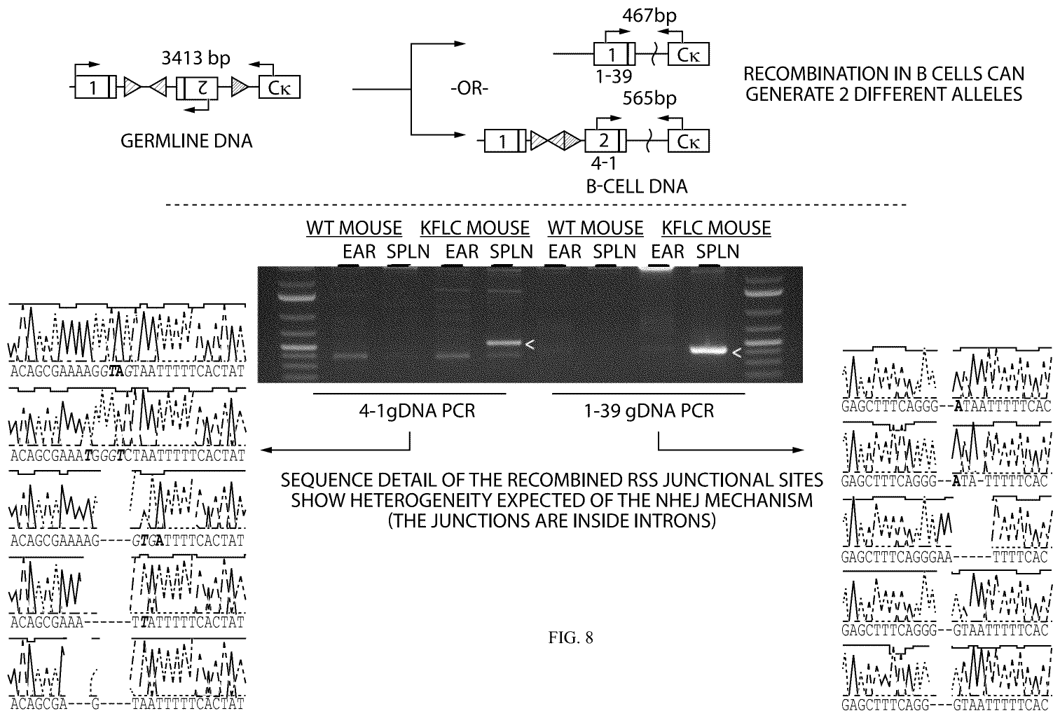

To demonstrate that recombination occurs in B cells to generate RSS-recombined fixed light chain alleles, genomic DNA was prepared from either ear or spleen biopsy, under the assumption that a high frequency of B cells (and hence recombination) could be found in the latter compared to ear tissue. As seen in FIG. 8, standard PCR was carried out with gene-specific primers (see directional arrows above the transgene schematic). PCR amplicons of the appropriate size for the two recombined alleles were only found in spleen samples (as expected; see arrows on agarose gel photograph), and not in ear DNA samples where few to no B cells should be found. In addition, the amplicons indicated by arrows were gel purified and subjected to DNA sequencing per the sequence chromatogram insets; the results show that the junctional sequences resulting from RSS-mediated recombination are heterogeneous. This is an expected outcome for such junctions which ultimately resolve via a process of non-homologous end-joining (NHEJ).

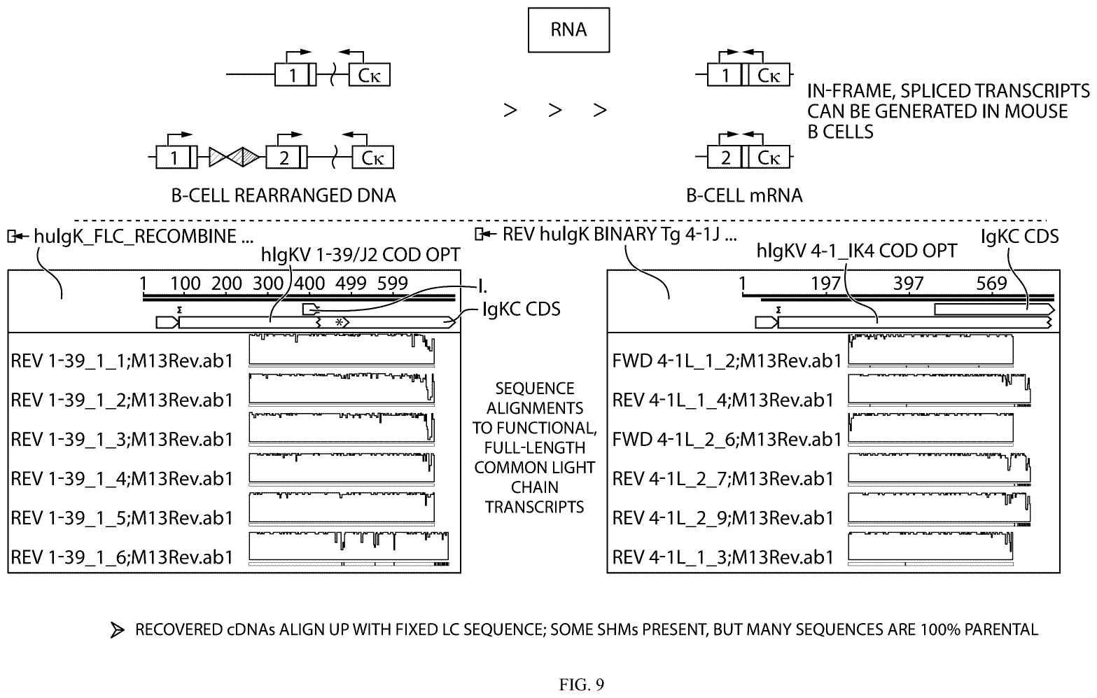

To demonstrate that such rearranged fixed light chain loci are capable of expressing properly splice mRNA transcripts, RT-PCR was carried out on spleen RNA samples from transgenic common light chain mice. As seen in FIG. 9, sequence analysis of individual cDNA clones, when compared to a hypothetical reference sequence created in silico, shows that in-frame transcripts can be recovered that correspond to the two fixed light chain alleles of fully human 1-39/J2/kC light chain or fully human 4-1/J4/kC light chain. Many of these sequences correspond 100% to the reference sequence, while others can vary due to occasional somatic hypermutation events.

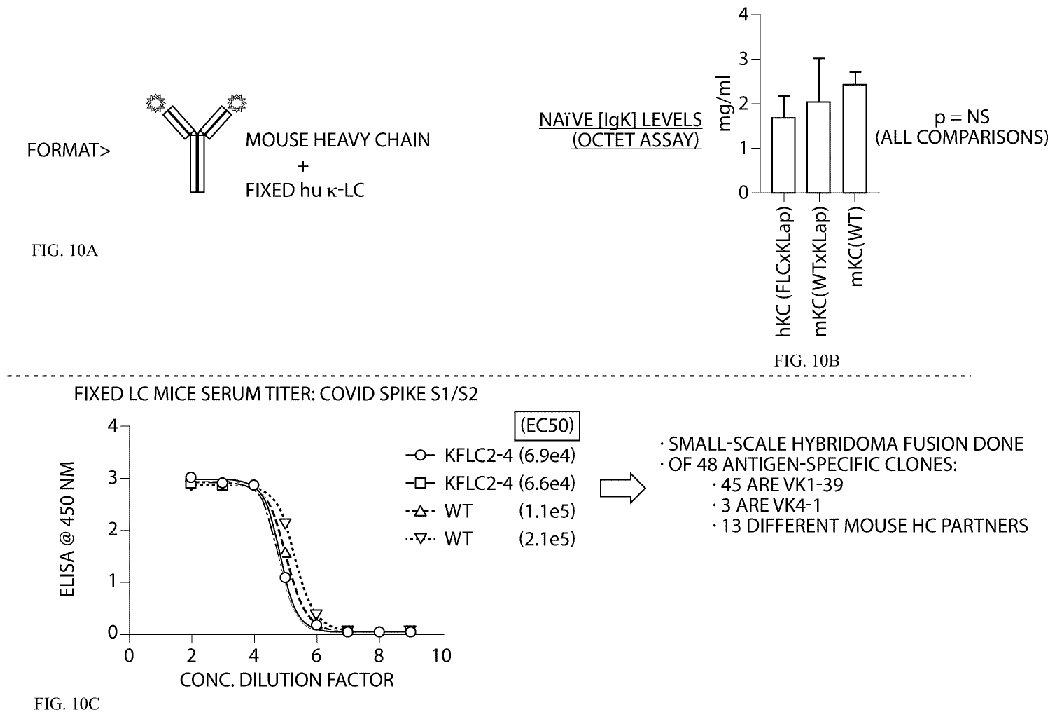

In order to establish that the recombined and expressed fixed light chain alleles can participate in a normal immune response, transgenic mice were immunized with COVID-19 spike proteins, and serum titers determined by ELISA. Prior to immunization, mice were bled to determine baseline levels of human k light chain expression. Three different mouse genotypes were analyzed. The first was a compound heterozygote consisting of one fixed light chain transgene allele and one null allele for mouse kappa LC (so-called KlaP, lacking all Vk and Jk sequences); the second was a compound heterozygote consisting of one wild-type mouse kappa light chain allele and one null allele for mouse kappa LC (KLaP); the third was a normal wild-type mouse. As can be seen in the results of FIG. 10A-FIG. 10C, naïve mice prior to immunization were indistinguishable for serum Ig kappa levels, using a non-species specific anti-kappa detection reagent. Following immunization, robust titers against COVID spike proteins were detected via sandwich ELISA using a first-stage capture of COVID spike followed by second-stage detection of all kappa light chains (non-species specific). The mid-point determination (EC50) of serum titer dilution showed that the fixed light chain-containing mice showed robust titers, and only differed from a wild-type response by an approximate factor of 2-3. Hybridoma fusions of the immunized mice followed by selection of antigen-specific clones yielded 200+ antigen specific hits; a subset of 48 randomly selected clones followed by sequence determination of the light chain component revealed that 45 of these were specific to the 1-39JK2 allele and 3 were specific for the 4-1JK4 allele. From the entire group of 48 clones, 13 distinct mouse VH heavy chain partners were identified. Together these results demonstrate that the binary transgene system is fully functional and can serve as a source of common light chains to facilitate bispecific antibody construction and therapeutic development.

### Example 2: Preparation of Human Lambda Binary Common Light Chain Constructs

In this example, the preparation of transgene constructs for expressing one of two alternate “fixed” human lambda light chains is described. Following on the approach described in Example 1 to create fixed light chain mice for the human kappa light chain, an analogous exercise was conducted to design and create a fixed human lambda light chain transgene. There were several steps to this process:
(i) Determination of the frequency of VL usage in humans, with the idea that highly expressed alleles are well tolerated, and support pairing with a wide array of potential heavy chain variable domains;
(ii) Combination of the VL usage data with JL usage data, to define specific VL-JL junctional species that can be exploited to make transgenes for a knockin;
(iii) Creation of in silico versions of the fixed or pre-recombined human lambda alleles (cassettes), in the context of the human genomic sequence surrounding them; and
(iv) Assembly of the cassettes into a functional transgene that can be used for gene targeting (i.e., also contains constant region, enhancers if needed, and engineered sites to enable site-specific gene delivery or knockin).

### Lambda Light Chain V Region Selection

The frequency of expression of lambda V regions in human populations was used as a starting point for lambda light chain V region selection. The IMGT® database provided information on V lambda frequency, leading to the determination that Vλ 2-14 and 3-19 were the most frequently used, although 3-21, 3-1 and 1-51 were also observed at high frequency. IGVL frequency as reported in human SARS2 patients (J. Exp. Med. (2022) Vol. 219, No. 9 c20220367) was also examined. Furthermore, Vλ and Jλ frequency in the human naïve repertoire also was analyzed based on data disclosed in DeKosky et al. (2015) Nat. Med. 21:86-91. Analysis of the data reported in DeKosky et al. led to the identification of the three top Vλ-Jλ pairs for each of three patients studied. The dataset was sorted for frequency of V Lambda segments and subjected to pivot analysis. Based on this analysis two candidate VL-JL pairs were identified as VL2-14/JL2 and VL1-40/JL2. The use of the J2 segments for both VL-JL pairs of the binary construct, however, would create near-identical CDR3 domains from each. Since one of the features of the binary fixed light chain approach is that the transgene provides a “choice” between two difference lambda light chains, and thus sequence divergence is an important consideration, a second VL-JL choice was made based on divergence parameters. For this reason, a non-J2 segment, such as a JL1 segment, can be used in one of the two VL-JL pairs of the final construct. Accordingly, VL-JL pairs selected for use in the binary fixed lambda light chain transgene were VL2-14/JL2+VL1-40/JL1.

Selection of the VL2-14/JL2 DNA sequence: Since there are many possible junctions between the VL-JL segments, to select the particular VL2-14/JL2 DNA sequence for use in the transgene construct it was helpful to rank different junctional pairs. Based on the DeKosky dataset, the identified CDR3 region was analyzed, as it contains the VL-JL junction within it. From the thousands of VL2-14/J2 LCDR3 sequences returned from each of the three donors, the most frequently recovered LCDR3s were identified and an alignment was run using commercially-available Geneious Prime software to generate a consensus sequence. Additionally, the CDR3 sequences were translated to derive the amino acid sequences and an alignment was run to determine the most common amino acid sequence of the CDR3. The LCDR3 sequence analysis led to the following consensus sequence (in which the sequence extends all the way to the end of the J segment): CSSYTSSSTLVVFGGGTKLTVL (SEQ ID NO: 6). This sequence was subjected to pBLAST, which confirmed it is commonly found in the NCBI database. The full-length variable domain of VL2-14/JL2 was also subjected to pBLAST, which revealed the full variable domain sequence also returns BLAST hits. Overall, the sequence analysis of the VL2-14/J2 combination confirmed it was found in GenBank and was likely to be expressed and active.

Selection of the VL2-1-40/JL1 DNA sequence: A similar exercise to the analysis of the 2-14/J2 pair was carried out on the 1-40/J1 pair. Nucleotide sequences from the DeKosky dataset were aligned to determine a ‘consensus’ LCDR3 from VL1-40/J1 transcripts. The nucleotide sequences also were translated and an alignment prepared of the translated sequence. The LCDR3 sequence analysis led to the following consensus sequence (in which the sequence extends all the way to the end of the J segment): CQSYDSSLSGYVFGTGTKVTVLG (SEQ ID NO: 7). The LCDR3 sequence, as well as that sequence incorporated into the full variable domain, were subjected to pBLAST, which confirmed the sequences were readily found in the NCBI database. Overall, similar to the VL2-14/J2 analysis, the sequence analysis of the VL1-40/J1 combination indicated that these are likely to be well tolerated and commonly found in human lambda light chain repertoires.

### Construction of the Fixed Light Chain Transgene

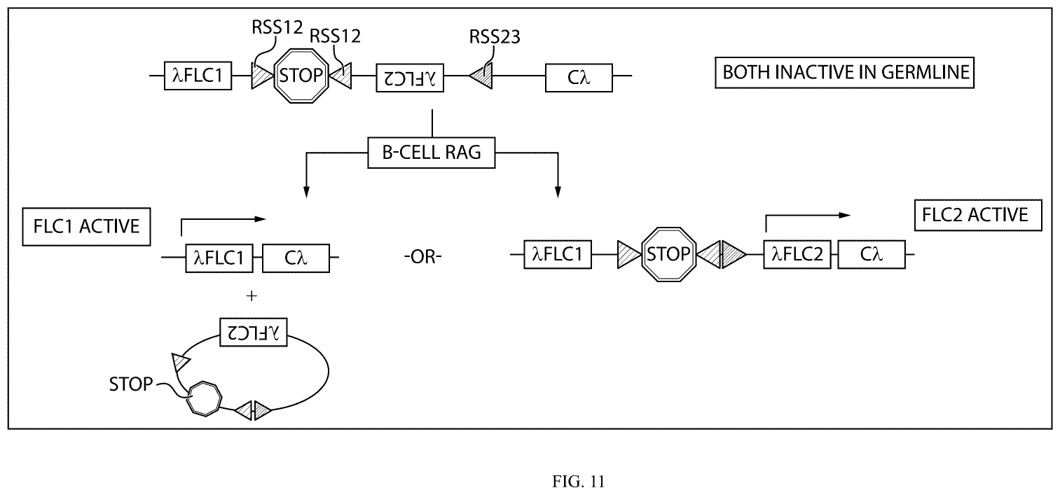

In terms of the overall strategy, the “binary” approach for a fixed light chain mouse has been validated with the human kappa light chains, as described in Example 1 and generally outlined in FIG. 2. Based on the success of the kappa fixed light chain model, a lambda fixed light chain model is created by substituting the coding sequences identified in the lambda light chain V region selection described above for the prior kappa coding sequences. This leads to a lambda binary fixed light chain construct comprising two lambda variable region cassettes (λFLC1 and λFLC2) arranged in opposite orientations, with both being inactive in the germline and one of the two cassettes becoming active upon RAG recombination, as illustrated schematically in FIG. 11. As in the kappa construct described in Example 1, the green and yellow triangles represent Recombination Signal Sequences (RSS), which are normally acted on during recombination to join a given light chain V segment to a given light chain J segment. In that context, the RSS sites are used to join coding sequences to one another; in this transgene context, they are placed in non-coding regions, but still intended to carry out the same recombination/joining function.

The two lambda fixed light chain cassettes are designed to “pre-rearrange” the Vλ and Jλ segments so there is no CDR3 variability or junctional diversity in that region. Moreover, given the success of the prior kappa approach, much of the original kappa light chain non-coding transgene sequences are used to create the lambda transgene. Furthermore, the lambda transgene can be delivered site-specifically to the mouse kappa light chain locus, as described herein (e.g., in Example 1 and FIG. 6), such as by Recombinase-Mediated Cassette Exchange (RMCE). Alternatively, CRISPR-based recombination methods can be used to deliver the lambda transgene to an endogenous light chain locus, such as the kappa locus.

Mice containing the lambda binary fixed light chain transgene can be bred to homozygosity. Mice can be cross-bred with a full-diversity heavy chain mouse (e.g., a humanized or human HC transgenic mouse).

Functional validation of the binary fixed light chain mice can include analysis of genomic DNA to show that recombination occurs in B cells to generate the two different light chain alleles, RNA analysis to show in-frame, spliced transcripts can be generated in mouse B cells and examination of protein levels of the lambda light chains. The titer and immune responsiveness to test antigens can also be examined and compared to wild type mice.

### Summary of Sequence Listing

| # | SEQUENCE |
|---|---|
| 1 | CQQSYSTPYTF |
| | (VK 1-39 + JK2 most common LCDR3) |
| 2 | CQQYYSTPLIF |
| | (VK 4-1 + JK4 most common LCDR3) |
| 3 | MDMRVPAQLLGLLLLWLRGARCDIQMTQSPSSLSASVGDRVTITCRASQSISSYLNWYQQK |
| | PGKAPKLLIYAASSLQSGVPSRFSGSGSGTDFTLTISSLQPEDFATYYCQQSYSTPYTFGQ |
| | GTKLEIKRTVAAPSVFIFPPSDEQLKSGTASVVCLLNNFYPREAKVQWKVDNALQSGNSQE |
| | SVTEQDSKDSTYSLSSTLILSKADYEKHKVYACEVTHQGLSSPVTKSFNRGEC |
| | (VK 1-39 + JK2 optimized allele) |
| 4 | MVLQTQVFISLLLWISGAYGEIVMTQSPDSLAVSLGERATINCKSSQSVLYSSNNKNYLAW |
| | YQQKPGQPPKLLIYWASTRESGVPDRFSGSGSGTDFTLTISSLQAEDFAVYYCQQYYSTPL |
| | TFGGGTKVEIKRIVAAPSVFIFPPSDEQLKSGTASVVCLLNNFYPREAKVQWKVDNALQSG |
| | NSQESVTEQDSKDSTYSLSSTLILSKADYEKHKVYACEVTHQGLSSPVIKSFNRGEC |
| | (VK 4-1 + JK4 optimized allele) |
| 5 | GCGGCCGCATAACTTCGTATAGCATACATTATACGAAGTTATGGCGCGCCATTCGCGACTC |
| | GAGCGCGCCGCACACAAAAACCAACACACAGATCATGAAAATAAAGCTCTTTTATTGGTAC |
| | CGAATTCGCCAGGGAGCTCTCAGACGTCGCTTGGTCGGTCTTTATTCGAACCCCAGAGTCC |
| | CGCTCAGGCACCGGGCTTGCGGGTCATGCACCAGGTGCGCGGTCCTTCGGGCACCTCGACG |
| | TCGGCGGTGACGGTGAAGCCGAGCCGCTCGTAGAAGGGGAGGTTGCGGGGCGCGGAGGTCT |
| | CCAGGAAGGCGGGCACCCCGGCGCGCTCGGCCGCCTCCACTCCGGGGAGCACGACGGCGCT |
| | GCCCAGACCCTTGCCCTGGTGGTCGGGCGAGACGCCGACGGTGGCCAGGAACCACGCGGGC |
| | TCCTTGGGCCGGTGCGGCGCCAGGAGGCCTTCCATCTGTTGCTGCGCGGCCAGCCGGGAAC |
| | CGCTCAACTCGGCCATGCGCGGGCCGATCTCGGCGAACACCGCCCCCGCTTCGACGCTCTC |
| | CGGCGTGGTCCAGACCGCCACCGCGGCGCCGTCGTCCGCGACCCACACCTTGCCGATGTCG |
| | AGCCCGACGCGCGTGAGGAAGAGTTCTTGCAGCTCGGTGACCCGCTCGATGTGGCGGTCCG |
| | GGTCGACGGTGTGGCGCGTGGCGGGGTAGTCGGCGAACGCGGCGGCGAGGGTGCGTACGGC |
| | CCGGGGGACGTCGTCGCGGGTGGCGAGGCGCACCGTGGGCTTGTACTCGGTCATGGTTTAG |
| | TTCCTCACCTTGTCGTATTATACTATGCCGATATACTATGCCGATGATTAATTGTCAACAC |
| | GTGCTGCTGCAGGTCGAAAGGCCCGGAGATGAGGAAGAGGAGAACAGCGCGGCAGACGTGC |
| | GCTTTTGAAGCGTGCAGAATGCCGGGCCTCCGGAGGACCTTCGGGCGCCCGCCCCGCCCCT |
| | GAGCCCGCCCCTGAGCCCGCCCCCGGACCCACCCCTTCCCAGCCTCTGAGCCCAGAAAGCG |
| | AAGGAGCAAAGCTGCTATTGGCCGCTGCCCCAAAGGCCTACCCGCTTCCATTGCTCAGCGG |
| | TGCTGTCCATCTGCACGAGACTAGTGAGACGTGCTACTTCCATTTGTCACGTCCTGCACGA |
| | CGCGAGCTGCGGGGGGGGGGGGAACTTCCTGACTAGGGGAGGAGTGGAAGGTGGCGCGAAG |
| | GGGCCACCAAAGAACGGAGCCGGTTGGCGCCTACCGGTGGATGTGGAATGTGTGCGAGCCA |
| | GAGGCCACTTGTGTAGCGCCAAGTGCCCAGCGGGGCTGCTAAAGCGCATGCTCCAGACTGC |
| | CTTGGGAAAAGCGCCTCCCCTACCCGGTAGCACGACTCGTACGTACGTACGAAGTTCCTAT |
| | TCTCTAGAAAGTATAGGAACTTCCACTGTGGCAGGTCAGCACCCCTGTTTATGTTCCTGTC |
| | ACCTGCCACAGCCTTGACTCTCATAACCAACAGGAAAATGAGGAGGTTCTAGGGCCCTGTG |
| | AGTAAAAAACTGGGATGATAGGGAAAGGAGAATGGAATCTCATCTGAATCCTCCTTCCTTG |
| | CCTACATTTGTTTAAATTTATTGAGCAAAAGGGCCAGACTACTGATTATTTCTGGCAAAAC |
| | ATGTTGAGTACATTTTAGGGTTTAACAGTTTTGGGTACCTTTCAAAGAAAATATTTGGTTA |
| | TATGTAAAATTGGTATTTTCCCACTTTTTAAATTCCTACTTCTCCTGTTTGCCATTCTTCT |
| | CCACTCCATGAGACAGTAGAGACAGGATTATTCACTAATTCTCCTCTGGCGGAGCTGGCTG |
| | AGGACAATCAGTAAGATCTTGGTTGTGAGTGTCAAATAGATTTTGTAATTTCATAGCAGAC |
| | GCAAGTTCCTAATACTAAAACTCTTTGATTACAATTACCTCTTGCTGATAGAAAAAGGGAG |
| | TTCTTGAAATTTTGAAAGTTGGTTTTAAAAATAAAATGCATACACTGGAAGATGCAGTATA |
| | CTAAAGATGTAGTATTTTCCAGGGATCACTGAGAAAATACAGGATGAGGTAAACAGTTTTA |
| | TTTTCCAAAGTTCAGAATTTGAGATTGGGCAGACTGCAGGAATGGGAGCATGAAGAGAACA |
| | TAAGAGAGATCAGCTATTGTTCAATTACACTGCCCTTGATTATGGTGGGTGGGGATGTGGC |
| | TGGTGGTGGTGATGGCAGTTGATGATGTGGATCCAAAAAGGGGCCAAATGTGTTACTTGTG |
| | AAGAACCACAGAGTTGAAAGCACTGCTGCATGGCTTCCTGGGTGGAGCCAAGTCTTCGCAC |
| | TTTCCAAACTTTCTCTCTCCTTTATTACTCACATGAAACTGCCCTCTCTAGTATTATGGTG |
| | GAAAAAGCATTCTGCACCAGCTATTTTCATGGGAGTATGGCCAAGGATAATTATTTTTACT |
| | ACTTATGGATTTTTAAAATCCAGGACAGATATAAATCCTAATACCAAAATAGTTTGATTTG |
| | CCTCAGTTGCCTTTTGCTGACTGAAAATAGAGTTCTTCCAATTCCAAAAGTGGGCTTTGAA |
| | AATAGACAAAATAATTCAGATGGAGAACATAAAGTTTACATAATGTACCACAGGAACAATG |
| | CAGAATTATATGAGATTTTTATTTCCTTCTCAAATTCTTAGAATTTTAAAAATATTTTACT |
| | GACATAGCATTTTAAAGAGAAAATTTTTAGTGCTATGTTGTCATAAGAAAATGATTCAAAG |
| | AATGAATAAATGCATTATTTTTATATGATACTATTCCTGAAAATAAATCTGAATCCTCTAT |
| | TTTAGTTGTTAATGCATAGAAAAATTATGCTCTTAATATATTCCATTGACAATAGTGCTCT |
| | AAATTTATATGCTTCCTTAATTTGAGGGCTACAGTCTGATAAATATCTATCTACATTTTGT |
| | CATGCAACTTTTAAACCTAACAGAAATGCTTTCGTTAAAAAAGCAACAGTGCTTTCTCTAC |
| | CATAATGCTAGAGAGGGCAGTTTCGTGTGAGTGGTAAAGGAGAAAGTTTGGAAAGTGGGAA |
| | GACAGCAATTTTGCAATATGACCAAGTGTTATCATGCAAAAAACTATCTCCAAACCAGTCA |
| | TTTCTAACATGTATTTCACTTGTGTTACCACTAATCTGCATCCATGATCTGATCATTCTGC |
| | CAAATAAATTTACACGAACCCGAGTAATGAGTGATGTCTGTCACATGTGATAACACGGTGT |
| | AGAGACACAAAAGACTGAATCAACAGAACAAGATCCTGATACAGGCTAAGGGTTTAAATAA |
| | TACTTGAGATGATTTATTTCAAGATAACAGATTCCATAGGAAGTGCAATTAAAATTCCAAT |
| | GTGTCATTTCTGTTAGGTTTAAAAGTTGCGTGGCAAAATGTACATAGATATTTATTAGACC |
| | GGAGCTCTCAAACTAAGAAATATGAGACACTCAATATGTTATTGAAAAATACTAATTAATG |
| | CATCATTGACCCTATATTTTATTACATAATGCAGTAAAAGAAAATAAAATGTAAGTAGCAA |
| | GAGCACGAGGTAAGCAATGCCATCGTCACCTTTAGCCCTCCTGGGATTGACAGCACCTAGT |
| | CACCTTGAGTTTCTGCTTTTCTGCGAGACAGGAGATAAAATGAAAACCCATTCAAGGTAGT |
| | TAGTTATATTTTGGAGAAAGCAAACAAAGATACCACCTACATGATGCTGATATCCCAAAGG |
| | CATATATTCTCAAGTCAAAATAGTGAAAAGTATATGATCCCCAAAACTGAAAGGGGAGAGA |
| | TACAAGGAGAATCAGAGCATGATCAAATTTAATTACAAAGAAGCCTCATAATATGGTGAAC |
| | TAAATGTAACAAGGTTTCTTTGTCTGTTGCCATGGCAGTGCAGAGGCAGGCAGGTGGCCTT |
| | GGTGGTGTAGGTGGCTCTGCTCCGTGAGGTCACTCCGGTGGGCAGGAGGCACGAACACCCT |
| | GAGAACACAGCCTTCCTCCTTCCTCACAGTCATTGCCTGCCCAGCCATCCTCAGCAGCATG |
| | AGGTGGGACAGAGTGGAGAGAAAGCTGTTTTCTTCTAAAGACTAAAAACAAAAACAGAAAA |
| | AAAAAAAACAAACCTGGAGCTTTCCATCAGTGACAAATGTACTTTTGACTCAATCACACAT |
| | TTGAGAAGTTTTCCAGTGAGTGGATCTGCAGATAAACCCACATTCGTTGTTTGTTTGTTTT |
| | AATCTGAAAATGTGTTTACATGATTCTTGAAAATATTTTTTGACTGTAAACTTATATTTGT |
| | AGTAGCCTATTTGAAGTTATCATTTACTATTTCATAATTGCTACTAAAAAGTTATTGTTAA |
| | AAAAATCTGTGACTCTAACTGTTCTTGTTTGAAAGGAATACGTCTTTTTAAGACACTCAGA |
| | CTCCTTTTAAGCTCCTCATTTGGCCCTCCTTTGTTTCTGATTCAATGTATTGTTTAATTTG |
| | TTATTTATGACTAATTAATCTATTAATTAATTTTCACAATCACAGAATCAAATGTCCTATA |
| | AGTTGCTATGTCAAAGACCTGCCTGAAGATGGCACACATGCTCCACAATGAACAAAGGACA |
| | GCACCATGGTCTCAGGAACACTGGGGAAGTAGGAGTGCTGGTGTCCCATTATCAACAGGGA |
| | GCCCTAGAGTTAGAGTCAAATCTTCCCTGTGACCTGGGCACCTGGGAGGAGCCACCCGTGT |
| | GCTGAGCTGTGGGAACCTGCCCCATGCCCTGAGACTGGAAGCACGGCCTTGGCTGTGTCCC |
| | ACCTGCTATGGACTGAATTGTGCCCTCAGATTCATGTTGAAACCCTAATTTTCAATGTGAC |
| | TGTAGAAATCAGAACCTCTAAAGATGTAATTGAGGTCATGGCAGGGGGTGTCCCTGATCCA |
| | GTAGGATTCATGATTTTGTAGGATCCAGAGACCTCTCCTTTTTCTTCTCTCTCTCTAACCG |
| | CCTTCCCCTCCATGGAAAGGCTGTGTGAAGACATGGTGAGAAGGTGGCACCTACAAACCAG |
| | GAATAAGGTCTTTAGCAGAAAACAAACTCTGCTGAACGTCGATCTGGGATATTCCAGGCAC |
| | CAGAAATGTGAAAATTAAATTCTGTGGTTTAGCCATCCAGCCCCGTGTTACGGTGTGGCAG |
| | CGGAAGCTGACTCATCCATCTTCCCCACCCTCTGTGAGCAGATCAGCTTCAGGAGGCCCTC |
| | GTGGACATGGGGTCCCAGCTTTGCTCCTCTTCCTCCTGTTTTTCCAACTCTCTGGTGAAGA |
| | GGGAGGACTCAAGCTTCATCATCAGTTTCTGTGCATTAAACATGAATGTTTCCTTCAAGAT |
| | GAAGTTTTTAGCCCATTTTGCTTTCTCAGAATTTAATCAAACTGAATGAAGTATGTACTTA |
| | CAATTAATATTTGGGGATGTTCATATTTGTTCCCCCCTTATATGACAGTTGGGATATTGTG |
| | TGGTGCTCATCTCCAGGCCCCTCCCTGTGTTCCAGGAGACAGGGTCACTGTCACCAGCAGA |
| | GCCAGTCAGGGAAATAACAGTGTCTAGCCTAGCTCCTTGTGAAATAAGGGCTGACACTGAG |
| | CTCCTGCTGGCTCCCACTGCTCCCTGGACACGATGCCTGCCTGGGTCAAGGGGGTGAGTCT |
| | GGACAGACGTCACTCTGGCCATCAGTAGCCTAACTACCTTCATGACCTCCCACTGTGAACA |
| | GAGTCCCGGAAGCTGCTGGAGCCATGAAAGTTGGACAGAGAAATCCCACATCACTGCAGTC |
| | AGAGGGGGGCTATGAAAAGACCGTGGGGGGGTTTCATACTATGACCACCCAGCACTGAGCC |
| | ATGGCTGCCACTCTGTCTGATGGGAGCCCCCAGGGGAAGATCCACTCACACTGTGCTCTAG |
| | AGGTCGTTTTTGTCACCATCTTTGTTCTAGCTGGTTTGGGCCAGTTTCTGTAGTGCATCCT |
| | GTTTTGTCCAGATCCTGTTCTGGTCAGCGTTGTCATGACCAGTGTTGTGATCAGTGCTCAG |
| | AATACAAGCCCTGATGATCTCCTACCTTATACTCACTGCCTTCTGTGAACCAGATGTTCTG |
| | ATAAGGGTCCTGTTGGATCCTACTCGAATCAGTTGCCACACAGACCCTCACTGAGGGCTGA |
| | GGGCCACAGACATCTGAAGATAAACAGAGGTCCAGAGAATGATAGCCTGTGACTGTCCTCT |
| | GTAAAGACAGCTGCTCCCCAGATGGCTGAGGGCTGTCTTTGGCTGTGTCCTTCTTTCTGAA |
| | TGATAATGAGGGATAGAGCAGGTCTCTAAGCAAAACCTCAGCAAGGCCTTCCTATGTGTGC |
| | TGCTCTAACCTGGACATAGGTGGCACTGGATACACTTAGGGAAGTGAGGAAATTTATAGTC |
| | ACAAGGAAGAGAGAGAAAGGAGGAGAGAGAGAGAAGAGCCTGTGATGTGTGTATAGTACCA |
| | ACACTGATAGTATGTTCTTTAATGGTTTATTGTTGAGTATGATGCTCAAATGCTCAGGTTC |
| | TATTCCATGCAGCCGATACATATACCTTATATAGGAAAAGGGCCTTTGCATATATAAATTA |
| | AGGATTATGAATTGGGCAGATTATCCTGTATTGACCAGGTGGGCCCTAAATGGGCCTGTCT |
| | TTATTAAATGTCTGTCTATCTCTCTTATAAGGACATCACGATTGTCCTTATAAGATAGACA |
| | TAGACAGACATTTTAACAAAGTCTGCAGAAGAGAAGGCAATTTGAAGACAGTGGCAGAGAT |
| | TGAAGTGATGTGGACTCAAGCCAAAGAAAGTTGAAGCCACCAAAAGCTGGAAGAAGCCAAA |
| | AAGTAGACTCCCCGCTGGAGTCCCTGCAGAAGCTTTGATGACAGCCTGCTCTTGACCCCTG |
| | AAACTAGTGCTGGACTTATGGCCTCCAGAACTATAGGAGGAGAATATATTTCTGTTGCTTT |
| | AAGCCACCAAAGTTTTAGTAATTTGTTATAGCAGCCCAAGAATTCTAATAAAAATGGGGCT |
| | TAGGATAAGTCCAGCCTAAAGGTAGTATGATGATTTGCACTCTCCACCTTCATTTCTCTAA |
| | TGTTACACATAATTGGTTAGAAGAAGATTTTTATGATGGAAACATTGTACAGGAAGCCACC |
| | CAGTATATACAGGGGCATCTGTTAGTTACAGAATAAATATTGACAGTTCTTAGTTGAAAAT |
| | GACATCTGAGGCTGGGCTTGGTGGCTCACGCCTATAATCCCAGCACTTTGGGAGGCCAAGG |
| | CGGGTGGATCACAAGGTCAGGAGATGGAGATCATCCTGGCTAACACAATGAAAAATCTGAA |
| | AAATACAAAATTTTTCAGAAATTCTGAAAAATACAAAAAATTAGCCAGACATGGTGGCACG |
| | TGCCTGTAGTCCAAGCTACTCGGGAGGCTGAGACAGGAGAATCCCTTAAACTCGGGAGGTG |
| | GAGGTTGCAGTGAGCCGAGACCACACCACTGCACTCAGGCCTGGGCAAGAGAGCAAGACTC |
| | TGCCTCCAAAACAAACAAACAAACAAACAAACAAAAAACCCACCAAACCAGGACTTTTTGA |
| | ACTCAGCTCTGAACCAAGTGGACCTAATAGACATCTACAGAACTCTCCATCCCAAATCAAC |
| | AAAATATACATTCTTCTCAGCAGCACATCGCACTTATTCTAAAATTGACCACAAATGCCTT |
| | ATGTAAATGACGAGTTGATGGGTACAGCAAACCCATATGGCACATGTACACCTATGTAACC |
| | TGCAAGTTGTGCACATGTACCCCAGAACTTAAAGTGTAATAATACAAAAAAATGACATGTG |
| | ACTAGTAGTATCTTATCTAGAATCTTCATTCTAAGATACTCAAGGACGCATAAAAGGGACC |
| | CTAAGTAGTCTTTTCATACATATATATGCACATATATATGTATGAAAAGCAGTCTTTTCAT |
| | CAACTAGAGAAACCCTCAGGACAGCCCTTAATACCCTTGGTGATACATTTCAGATGAGTAA |
| | ACTGTTATCAGAGCCCGTAGTTGAAACTATTCAACAGAGATGGTTTGCCCAAAGATATGTG |
| | GTCAGCAATTGTCAGGGCTGAGCTTGGAACCCAGGTCTGCATAACCTTAAATATGTTGCTT |
| | CCACATGGCCACGTTTGTTTCATATACGATTGAATGGCCTTTAAATTCAAAGAAGAGACAA |
| | AGCCAGAAGAGTGGTGTGAAATTCTCAACACAAGCTCCCTGCTACCTCTACACCTTACCGT |
| | GATTACTCCAATTATAAACTCAGGCCCTCATGCAGTTTTGTCTACAAAGCAAAACTTCCTC |
| | AAAGTCTTTACAAATACTAAATGTCTTTCTTTCAGATTCGAGGGCAAGAGCACATCTTGCA |
| | TTGCCCTGAACACTTTGCATCTTTTCTACCATTCTCATCTTTCTGTCCCAGTCCTTCCTTC |
| | TCAAATGATGTCCTGTAAATCTGATTTCTCCCCCAATATGAAAACAAATGAACAAATATTC |
| | CCCTACTTTTCTCATATCCAGAGGATACAAGAGTTAATCACATATCCAGAGAGTACGAGAG |
| | TTAATCAAGGGATTTATGCAAGAGTGTTTACACATAACAAGGATTCTGGTGCTAGCCATCT |
| | TCACAGTGAAATTTTCTGTGTGTCTTGCTAAAATTGACACTAAAAAATGACAAGATAAAAA |
| | TATTTGGAAGAACAGAGGGCAACCATGCCCTTAAGGAGGTAAAAGACACCCCTGCCCCTTG |
| | TGTTAGTTTCCTACTCCTGCTGTAACAAGTTATCAGAATCTTACTAGTTTCATACAACACA |
| | AATTTATTATCATACAGTTCTGGCAGTGAGAAGTCTCACTGATTTATAATCAAGGTATCCG |
| | TAGTTCTATATTCCTTCTGGAAGTTCCAGGGGAGAGAATCTGATTCTCAGCTTTTCATCTT |
| | CAAAGATAGCCCCATGTTCCGGGCTGCCTGGCCCCTTTCTCCATCACTGAAGCATCCCTGT |
| | CCATTGTCCCTATTCCTCTCTGACTGTTACCCCCACTCCTCCCTATTATAAAGACCCTTCT |
| | GATGACGCTGTCTTTCCTAGATAATTCAGCTGTTTCCTAAATTTTCTGAATATCCCTATGC |
| | ATGAAAAAAAAAGAATTGGCAAGTATTCAGACTATACTTTCCAAGAATGAGGGTTTGTCCA |
| | CTGTTTTAGGTTGGATCTTTCAGGGACAATGATGCCCATGCAGGCAGCATATTTATAATGC |
| | ACAGTAAACACTAGGAGGAAACAAGGCAGTGAGAGAGGAAAGAGAGCAGCGATACCGAAAA |
| | TGTCCTCAGCGAGAAGCTACCACAGAGGATGAATGGAGATCAAGCCCACGTGGAAACATGG |
| | GAAAATGTCTCAGTATTTTTCCACCTAAGAAGGGAGGGAGATGGGGTATGTATACACCTCC |
| | CTGTCCTCACTGATTGAGGGCTTTCCGAGAGGATGCTCATTCCAGGTGCTGTGATAGGCCA |
| | TGTGTACAGGCAGGGCTGCCTTCTCCAGCTTCAGATAGAGCAGTGAGGAAAAGATATGGCC |
| | ATGGGGGGTCAGCAGAAGTACAGCAAAGGGAAAAGGGAAAGGGTAGCAAGAGTGACAACTA |
| | TATTCACCCCCCCCACACACACACACACACACACGAAATTGTGTATTGCAATCCAGAACTG |
| | CTTCTCTCTGAACCTAAATCTTAGCAAGCAGTTTACCAGTAACTGCCCTTGAAATTCAGGC |
| | CCCTGGAAAGGAGCAGGGGGTTGTGTACAGGCTATACCACAGCAGTCTGCCCACCCTTAGT |
| | GATGCATGAGTAATGCTCCCTGGACTCCCCAGGTTCTAGTCTTCTCATGTCGATGTAGTTG |
| | ATTCCACTTCCCTTGCTGCACAACCAGGCTGGGATGCCTGGGCAGAGGCAGACATGTGAGG |
| | TATAGGGGTTCAAATCTGTTTCCAAGTTTTATCCAGCTTCAAAGCATTTCTCCGTGTACAT |
| | GAGCGGTGGCTTGACAGGAGATGGAGACTCTCTTTCCTGGATGTGAGGCAAGGAGGCAGGC |
| | GTCTGAGTCAGGATGATGTCCCTACTCACTGCTAAAGAGAAAAGTGGCTTTGATGGTGCAG |
| | GGCAGGGAAATGCACTGAGTGGTCGCCACCCTCACAGAAGAGAAAGTGTTCACTGACCTGG |
| | CCTTTCCCCAGGGCCTCTCCCTCCCATTGCTTTCCAGAAAGCCATGATTTTTGAGAGCCAC |
| | ACCTGAACACTCACAAACATTATGGTGGGAAAAGCAGATCAGAGCATTAGGCAAGTTGCAT |
| | TACCTTGGCCTTCTTCCTTTGGAGACAATTGATGTGGGGTTCTAGATTGACCCAGAGTTTC |
| | AAGTTTATCCTGATTCAGGCTTCAACAGCTGGAGGAAGAAACAGAGATGTTTTTTGAAGTA |
| | AACAGATCTAGCATTACTAATCAACCCTTCATACTGATGACCTATGGGAAATAATACCCAA |
| | GGGCAGAAAAATGGGCAGAATAAGGGGAGCCCCAAACCAAGACGAAGCTGCTGCCCATTGA |
| | GACCCTGGGTATTACAGAGACCTATAGCTCTGGATAATGGAAGATCTATGAGTGGCACAGG |
| | CGCTGAGGAATCACAGCATCATTATCGTGCATCTGCAGGGAATTGCTTGTAAATATACTGG |
| | TAATTACAAATGTTTAAGGTCACTACAAATACTTTGGAGTGTATTAAATATGCTTCTGATA |
| | AAGACTGTTTTTCTCACATGAAACAATGGGAACCATGTGACAATCACAGAGGTGTTGTTAC |
| | TATAGCAAAAGGGATTGTTACTCTCCACATCCCTTTAAGTAACTTGAAGGCCTGATAGACC |
| | CACCCTCTAAGACTTCATTAGACATTCCCTACGAATGGTTATACTCTCCTGTATACTCCCA |
| | ATACAACTCTAAAATATATTATTCCATATAGTCCTTAGGTTTGTATTAAAGTTTGACTTTT |
| | TTCCTTCAAAATATCTCTTGTCACAACAGCGGCTCTAGAGAGAAATACATTCCCTCCAGGC |
| | AAATCTATGCTGCGCTGGTCTGACCTGGGACCCTGGGGACATTGCCCCTGTGCTGAGTTAC |
| | TAAGATGAGCCAGCCCTGCAGCTGTGCTCAGCCTGCCCCATGCCCTGCTGATTGATTTGCA |
| | TGTTCCAGAGCACAGCCCCCTGCCCTGAAGACTTTTTTATGGGCTGGTCGCACCCTGTGCA |
| | GGAGTCAGTCTCAGTCAGGACACAGCATGGACATGAGGGTCCCCGCTCAGCTCCTGGGGCT |
| | CCTGCTACTCTGGCTCCGAGGTAAGGATGGAGAACACTAGGAATTTACTCAGCCAGTGTGC |
| | TCAGTACTGACTGGAACTTCAGGGAAGTTCTCTGATAACATGATTAATAGTAAGAATATTT |
| | GTTTTTATGTTTCCAATCTCAGGTGCCAGATGCGACATCCAGATGACGCAGAGCCCGTCCT |
| | CGTTGAGCGCCTCCGTCGGCGACCGCGTGACTATCACTTGTCGGGCGTCCCAGTCTATTAG |
| | CTCCTACCTGAACTGGTATCAGCAGAAGCCAGGAAAGGCTCCCAAGCTCCTTATCTACGCC |
| | GCATCATCGCTGCAATCCGGAGTGCCGTCCCGGTTTTCGGGCTCGGGTTCCGGCACCGACT |
| | TCACCCTGACCATTAGCTCCCTGCAACCCGAAGATTTCGCCACCTACTACTGCCAGCAGTC |
| | CTACTCAACTCCTTACACCTTCGGACAAGGGACCAAACTCGAGATCAAGCGTAAGTACTTT |
| | TTTCCACTGATTCTTCACTGTTGCTAATTAGTTTACTTTGTGTTCCTTTGTGTGGATTTTC |
| | ATTAGTCGGATGCCAGGGATCTAACAAACTTCATTCCCAGGTTAGGTACAGAGGAGGGGAA |
| | ATTGTTCCACAGGACGCTAGCTTGTGGCTAATTTTTAAGATTTCTAAATCAAAATAACTTC |
| | ATTGGGGGAAAGAGGCTTGCTGAGCTTTCAGGGACACAGTGTTACAAGTCATAACATAAAC |
| | CAAAACACCCTGACCCCTCTGCCTGGCATAGACCTTCAGACACAGAGCCCCTGAACAAGGG |
| | CACCCCAACACCTCATCATATACTGAGGTCAGGGGCTCCCCAGGTGGACACCAGGACTCTG |
| | ACCCCCTGCCCCTCATCCACCCCGCAGGTTCTCAACCCTCACCCCCCACCACGGGAGACTA |
| | GAGCTGCAGGATCCCAGGGGAGGGGTCTCTCCTCCCACCCCAAGGCATCAAGCCCTTCTCC |
| | CTGCACTCAATAAACCCTCAATAAATATTCTCATTGTCAATCAGAAATCTTGTTTTATCTC |
| | ATTTTTTCTTTTCTCACATATAATTCCTAGCCTTTCCTGGGTTCTCAATTTGTGGTGGGGT |
| | TTATGTTATGACTTGTAACACTGTGTTTCGCTGTTAATATCACTAACCTGACCGATGCAGA |
| | GAAAATCTTGCAATTGAGATGCCTCACTTAACTGGCTAGCGCTTGGCTGTTCCTTAAGATG |
| | AACTAATTTTCTATCCCTTACTCATCTGACTTTTTGAAAGAATCTGGTACTCTTTGGAATT |
| | GACCTGAGCTAATATCTCAAACACAAAAACGCTCCAAATTTAAAACCTTATAAGAAAAAGC |
| | ATTAGGAAAGTGCACTTACGCTTAATCTCGACCTTGGTTCCACCCCCAAAGGTAAGGGGAG |
| | TGGAGTAGTACTGTTGGCAGTAGTACACTGCGAAGTCCTCGGCCTGCAGGCTGCTGATCGT |
| | CAAAGTGAAGTCAGTGCCGGAGCCGCTCCCCGAGAACCGATCCGGCACTCCGGATTCGCGG |
| | GTCGAGGCCCAGTAGATGAGCAGCTTAGGCGGCTGTCCTGGCTTTTGCTGATACCAGGCGA |
| | GGTAATTCTTGTTGTTTGAGGAGTACAGCACGGACTGCGAGCTTTTGCAGTTGATGGTAGC |
| | TCTCTCTCCCAGGGACACGGCCAGGGAGTCGGGTGACTGGGTCATCACAATTTCGCCGTAC |
| | GCACCTGTAGTCAGTAATGGACAATGATTATACATAAACTTATACACGCTGTCTCAGATAT |
| | AATTGAAATATGTCATTTAAATTTGCTTTCTAGTAGTATTATAAGTCAGCAGATAATTGGA |
| | TCCATATTGGAAATAGTCATTATTTCCAACTATATCTTAATTTTTTTTATTTCTACACAGA |
| | TATTACTCTGAAAAGACTGTGGCACTTTTTAATTCCTCACCAGAGATCCAGAGCAACAGAG |
| | AAATGAAGACCTGGGTCTGCAACACCATCTTGCTGCCCCTGCCTGCCTGTTGTAGCTCAGT |
| | TCACAATGCAAACGGCCCGTTTATAAAATCCAAACAGCTGGTCGTGGCCTGGAGGGGCCTA |
| | TGCAAAAACAACCAGTGAGTACAAAAGCAAATTTCATGAGCAGTGGGTTGTGAAAGTACCC |
| | AATGTAAATCAAGAGCCAAAAATATCATCACAGAATGTGTAATTGTATGAATAGAAAAGAC |
| | AGAAGTCAACTTAGAAGCTCTAAAACTACAAACTGCTGAAAGATCTAATGACTAGGACAGC |
| | CTAGTAATTTTCATAGGGGCATAAATGTGAAACGCCTTGTGCATCGTAGAAGAAAGCAGAA |
| | GAGAAAGCATTCCCAATTTCTTAACTGCCTTTTACCTATATTAATCAGTAATATACTGGCT |
| | TTTACCTCTGTTAATCATAATAAACAAATTCTCAATAAATTTTATCGATACTCTTCAATGC |
| | CTGCTCAGCAACATTTTCCGAAGGCAGCTCAAGATATTAAATAACTCATAAGGGCCAACCT |
| | CCTATTGCAGCATTCTTTGGGATTTAACCAGTTTCCCAAGACTCTTTTCACAATGTTAAGA |
| | TGTTAGAAATAGATCCAAAACTAGGTGATATATCCCCTAGTAAAACTGTGAGGTCAAACTT |
| | GTCTGGCTAATGCTTCCATTTAAAAATTTCTCTTTCTTGATCCTTCATTGTATGTACACAA |
| | TAAATCAGGGGAAAACTTTAACTGAGTGAATCAAAGTATTCTCATTATTATAATAGGAGCT |
| | TCACACACACACAAAAAAATCAATTCTATTACTCTCAGCCTCAGTTCCTAAAGCCAAGTTA |
| | AAGTCCTGTTCTAAGATCATTGTTGCATGACCATATGTATTCCAGGTCTAATCTAAACTGT |
| | GGATAAATCCCAGCAGGACATTAGAGATTTTTGTGAGAGTAAGCATATAGGATTCAGGGTT |
| | TATGAGCTTTAGATTTTTCTTGTCAAAATGAATGAGAGTTGCCATATCTAAAAATTATTCC |
| | CAGATAAATAAAATTCACTACCTAGAATTAATTTATGCATATAAGTAGAAATGCTATCTCC |
| | CTTTTTACCATCCAAAGTGGAAAGCCTCATGGAACTAGAAATTAATATTAGAAAAATCAGT |
| | TAATAAAAGTATGTCATTTCATCAATTCAATAAGTTATAATAGCAAAAAACCATAATAAAT |
| | TATCACTTAAATGTCAATACATTTATAAACTATGGTACATAAATAGGATATTGAATAGCCA |
| | TTGATGCTCCTGATGAAAATTAGCAGGCAGTGATAAATGATAAATATGAAGCACATGTCAA |
| | TAAATAAAATAAGTTTTATGTAATTTAGGAGAAAATGGTGATAATGACACAAAATGTGAAT |
| | TATGGATGCATCTATAAAATTCTTTGTACATTTGTGAATTGTAAATATTTATCTTAGAGAC |
| | ATTATTACTTTGTATATGTTCCATTTGCTCACCTATATGTCCCAGTCTCCTTACAAATGCT |
| | ATGGCCAAAGAAATAGGCATACATACATCCTTTGCAGGCTGAGGCAGGAAAAAGATCTTAC |
| | GGAATTTTCCAGTCTATCCTTTATCTGTATAAGCAACTTAAGAGGCCATGTGCTCCAAATG |
| | GTGCAAATACAAGGGTTTTTGTTCAGCAAGACAATGGAGAGCTCTCACTGTGAGTAATTTT |
| | TCACTATTGTCTTCTGAAATTTGGGTCTGATGGCCAGTATTGACTTTTAGAGGCTTAAATA |
| | GGAGTTTGGTAAAGATTGGTAAATGAGGGCATTTAAGATTTGCCATGGGTTGCAAAAGTTA |
| | AACTCAGCTTCAAAAATGGATTTGGAGAAAAAAAGATTAAATTGCTCTAAACTGAATGACA |
| | CAAAGTAAAAAAAAAAAGTGTAACTAAAAAGGAACCCTTGTATTTCTAAGGAGCAAAAGTA |
| | AATTTATTTTTGTTCACTCTTGCCAAATATTGTATTGGTTGTTGCTGATTATGCATGATAC |
| | AGAAAAGTGGAAAAATACATTTTTTAGTCTTTCTCCCTTTTGTTTGATAAATTATTTTGTC |
| | AGACAACAATAAAAATCAATAGCACGCCCTAAGAAAAATCAGGGAAAAGTGAAGTGTACCT |
| | ATTTGCTATGTAGAAGAGGCAGCTTACTTGAAAATCAGCAGCAATGTTGTTTTTAGAGTCT |
| | GTAATAAGTAATAAACTCAAAAAGACACATTCTATAGGAATAAGGGCTTCACAGATAGAGC |
| | TCATTTTTTAAAAATCCAATTTGTACATTAGACTAAACGTGAAATTATCTCTTATTGTAAT |
| | GGTGGAAAGGTGGTTATTCCCAAAAGCTCAATCTCAAAGAAATGTGTTTAAATGAAAAAAA |
| | GTAAATAATTGCATTTTTTAATGACCGTGGGTCTGTGAAAAAAATAGGAAATATTTTAAAG |
| | AGTATGTTCTTTCATTATCCTCTGTTATTACTTGTCTACATTTTTATTCTGCCAAGAAGGC |
| | CGTGGCACCGCGAGCTGTAGACAGAGCCGCGGTCTTTCTCGATTGAGTGGCTTTGGTGGCC |
| | ATGCCACCGCGCTCTTGGGGCAGCCGCCTTGCCGCTAGTGGCCGTGGCCACCCTGTGTCTG |
| | CCCGATTGATGCTGCCGTAGCCAGCTTTCCTGATGCACAGTGATACAAATAATGCCACTAA |
| | GGGAAAGAGAACAGAAACGTAATGGGCGCTGGCGGCCGCATAACTTCGTATAGCATACATT |
| | ATACGAAGTTATGGCGCGCCATTCGCGACTCGAGCGCGCCGCACACAAAAACCAACACACA |
| | GATCATGAAAATAAAGCTCTTTTATTGGTACCGAATTCGCCAGGGAGCTCTCAGACGTCGC |
| | TTGGTCGGTCTTTATTCGAACCCCAGAGTCCCGCTCAGGCACCGGGCTTGCGGGTCATGCA |
| | CCAGGTGCGCGGTCCTTCGGGCACCTCGACGTCGGCGGTGACGGTGAAGCCGAGCCGCTCG |
| | TAGAAGGGGAGGTTGCGGGGCGCGGAGGTCTCCAGGAAGGCGGGCACCCCGGCGCGCTCGG |
| | CCGCCTCCACTCCGGGGAGCACGACGGCGCTGCCCAGACCCTTGCCCTGGTGGTCGGGCGA |
| | GACGCCGACGGTGGCCAGGAACCACGCGGGCTCCTTGGGCCGGTGCGGCGCCAGGAGGCCT |
| | TCCATCTGTTGCTGCGCGGCCAGCCGGGAACCGCTCAACTCGGCCATGCGCGGGCCGATCT |
| | CGGCGAACACCGCCCCCGCTTCGACGCTCTCCGGCGTGGTCCAGACCGCCACCGCGGCGCC |
| | GTCGTCCGCGACCCACACCTTGCCGATGTCGAGCCCGACGCGCGTGAGGAAGAGTTCTTGC |
| | AGCTCGGTGACCCGCTCGATGTGGCGGTCCGGGTCGACGGTGTGGCGCGTGGCGGGGTAGT |
| | CGGCGAACGCGGCGGCGAGGGTGCGTACGGCCCGGGGGACGTCGTCGCGGGTGGCGAGGCG |
| | CACCGTGGGCTTGTACTCGGTCATGGTTTAGTTCCTCACCTTGTCGTATTATACTATGCCG |
| | ATATACTATGCCGATGATTAATTGTCAACACGTGCTGCTGCAGGTCGAAAGGCCCGGAGAT |
| | GAGGAAGAGGAGAACAGCGCGGCAGACGTGCGCTTTTGAAGCGTGCAGAATGCCGGGCCTC |
| | CGGAGGACCTTCGGGCGCCCGCCCCGCCCCTGAGCCCGCCCCTGAGCCCGCCCCCGGACCC |
| | ACCCCTTCCCAGCCTCTGAGCCCAGAAAGCGAAGGAGCAAAGCTGCTATTGGCCGCTGCCC |
| | CAAAGGCCTACCCGCTTCCATTGCTCAGCGGTGCTGTCCATCTGCACGAGACTAGTGAGAC |
| | GTGCTACTTCCATTTGTCACGTCCTGCACGACGCGAGCTGCGGGGGGGGGGGGAACTTCCT |
| | GACTAGGGGAGGAGTGGAAGGTGGCGCGAAGGGGCCACCAAAGAACGGAGCCGGTTGGCGC |
| | CTACCGGTGGATGTGGAATGTGTGCGAGCCAGAGGCCACTTGTGTAGCGCCAAGTGCCCAG |
| | CGGGGCTGCTAAAGCGCATGCTCCAGACTGCCTTGGGAAAAGCGCCTCCCCTACCCGGTAG |
| | CACGACTCGTACGTACGTACGAAGTTCCTATTCTCTAGAAAGTATAGGAACTTCCACTGTG |
| | GCAGGTCAGCACCCCTGTTTATGTTCCTGTCACCTGCCACAGCCTTGACTCTCATAACCAA |
| | CAGGAAAATGAGGAGGTTCTAGGGCCCTGTGAGTAAAAAACTGGGATGATAGGGAAAGGAG |
| | AATGGAATCTCATCTGAATCCTCCTTCCTTGCCTACATTTGTTTAAATTTATTGAGCAAAA |
| | GGGCCAGACTACTGATTATTTCTGGCAAAACATGTTGAGTACATTTTAGGGTTTAACAGTT |
| | TTGGGTACCTTTCAAAGAAAATATTTGGTTATATGTAAAATTGGTATTTTCCCACTTTTTA |
| | AATTCCTACTTCTCCTGTTTGCCATTCTTCTCCACTCCATGAGACAGTAGAGACAGGATTA |
| | TTCACTAATTCTCCTCTGGCGGAGCTGGCTGAGGACAATCAGTAAGATCTTGGTTGTGAGT |
| | GTCAAATAGATTTTGTAATTTCATAGCAGACGCAAGTTCCTAATACTAAAACTCTTTGATT |
| | ACAATTACCTCTTGCTGATAGAAAAAGGGAGTTCTTGAAATTTTGAAAGTTGGTTTTAAAA |
| | ATAAAATGCATACACTGGAAGATGCAGTATACTAAAGATGTAGTATTTTCCAGGGATCACT |
| | GAGAAAATACAGGATGAGGTAAACAGTTTTATTTTCCAAAGTTCAGAATTTGAGATTGGGC |
| | AGACTGCAGGAATGGGAGCATGAAGAGAACATAAGAGAGATCAGCTATTGTTCAATTACAC |
| | TGCCCTTGATTATGGTGGGTGGGGATGTGGCTGGTGGTGGTGATGGCAGTTGATGATGTGG |
| | ATCCAAAAAGGGGCCAAATGTGTTACTTGTGAAGAACCACAGAGTTGAAAGCACTGCTGCA |
| | TGGCTTCCTGGGTGGAGCCAAGTCTTCGCACTTTCCAAACTTTCTCTCTCCTTTATTACTC |
| | ACATGAAACTGCCCTCTCTAGTATTATGGTGGAAAAAGCATTCTGCACCAGCTATTTTCAT |
| | GGGAGTATGGCCAAGGATAATTATTTTTACTACTTATGGATTTTTAAAATCCAGGACAGAT |
| | ATAAATCCTAATACCAAAATAGTTTGATTTGCCTCAGTTGCCTTTTGCTGACTGAAAATAG |
| | AGTTCTTCCAATTCCAAAAGTGGGCTTTGAAAATAGACAAAATAATTCAGATGGAGAACAT |
| | AAAGTTTACATAATGTACCACAGGAACAATGCAGAATTATATGAGATTTTTATTTCCTTCT |
| | CAAATTCTTAGAATTTTAAAAATATTTTACTGACATAGCATTTTAAAGAGAAAATTTTTAG |
| | TGCTATGTTGTCATAAGAAAATGATTCAAAGAATGAATAAATGCATTATTTTTATATGATA |
| | CTATTCCTGAAAATAAATCTGAATCCTCTATTTTAGTTGTTAATGCATAGAAAAATTATGC |
| | TCTTAATATATTCCATTGACAATAGTGCTCTAAATTTATATGCTTCCTTAATTTGAGGGCT |
| | ACAGTCTGATAAATATCTATCTACATTTTGTCATGCAACTTTTAAACCTAACAGAAATGCT |
| | TTCGTTAAAAAAGCAACAGTGCTTTCTCTACCATAATGCTAGAGAGGGCAGTTTCGTGTGA |
| | GTGGTAAAGGAGAAAGTTTGGAAAGTGGGAAGACAGCAATTTTGCAATATGACCAAGTGTT |
| | ATCATGCAAAAAACTATCTCCAAACCAGTCATTTCTAACATGTATTTCACTTGTGTTACCA |
| | CTAATCTGCATCCATGATCTGATCATTCTGCCAAATAAATTTACACGAACCCGAGTAATGA |
| | GTGATGTCTGTCACATGTGATAACACGGTGTAGAGACACAAAAGACTGAATCAACAGAACA |
| | AGATCCTGATACAGGCTAAGGGTTTAAATAATACTTGAGATGATTTATTTCAAGATAACAG |
| | ATTCCATAGGAAGTGCAATTAAAATTCCAATGTGTCATTTCTGTTAGGTTTAAAAGTTGCG |
| | TGGCAAAATGTACATAGATATTTATTAGACCGGAGCTCTCAAACTAAGAAATATGAGACAC |
| | TCAATATGTTATTGAAAAATACTAATTAATGCATCATTGACCCTATATTTTATTACATAAT |
| | GCAGTAAAAGAAAATAAAATGTAAGTAGCAAGAGCACGAGGTAAGCAATGCCATCGTCACC |
| | TTTAGCCCTCCTGGGATTGACAGCACCTAGTCACCTTGAGTTTCTGCTTTTCTGCGAGACA |
| | GGAGATAAAATGAAAACCCATTCAAGGTAGTTAGTTATATTTTGGAGAAAGCAAACAAAGA |
| | TACCACCTACATGATGCTGATATCCCAAAGGCATATATTCTCAAGTCAAAATAGTGAAAAG |
| | TATATGATCCCCAAAACTGAAAGGGGAGAGATACAAGGAGAATCAGAGCATGATCAAATTT |
| | AATTACAAAGAAGCCTCATAATATGGTGAACTAAATGTAACAAGGTTTCTTTGTCTGTTGC |
| | CATGGCAGTGCAGAGGCAGGCAGGTGGCCTTGGTGGTGTAGGTGGCTCTGCTCCGTGAGGT |
| | CACTCCGGTGGGCAGGAGGCACGAACACCCTGAGAACACAGCCTTCCTCCTTCCTCACAGT |
| | CATTGCCTGCCCAGCCATCCTCAGCAGCATGAGGTGGGACAGAGTGGAGAGAAAGCTGTTT |
| | TCTTCTAAAGACTAAAAACAAAAACAGAAAAAAAAAAAACAAACCTGGAGCTTTCCATCAG |
| | TGACAAATGTACTTTTGACTCAATCACACATTTGAGAAGTTTTCCAGTGAGTGGATCTGCA |
| | GATAAACCCACATTCGTTGTTTGTTTGTTTTAATCTGAAAATGTGTTTACATGATTCTTGA |
| | AAATATTTTTTGACTGTAAACTTATATTTGTAGTAGCCTATTTGAAGTTATCATTTACTAT |
| | TTCATAATTGCTACTAAAAAGTTATTGTTAAAAAAATCTGTGACTCTAACTGTTCTTGTTT |
| | GAAAGGAATACGTCTTTTTAAGACACTCAGACTCCTTTTAAGCTCCTCATTTGGCCCTCCT |
| | TTGTTTCTGATTCAATGTATTGTTTAATTTGTTATTTATGACTAATTAATCTATTAATTAA |
| | TTTTCACAATCACAGAATCAAATGTCCTATAAGTTGCTATGTCAAAGACCTGCCTGAAGAT |
| | GGCACACATGCTCCACAATGAACAAAGGACAGCACCATGGTCTCAGGAACACTGGGGAAGT |
| | AGGAGTGCTGGTGTCCCATTATCAACAGGGAGCCCTAGAGTTAGAGTCAAATCTTCCCTGT |
| | GACCTGGGCACCTGGGAGGAGCCACCCGTGTGCTGAGCTGTGGGAACCTGCCCCATGCCCT |
| | GAGACTGGAAGCACGGCCTTGGCTGTGTCCCACCTGCTATGGACTGAATTGTGCCCTCAGA |
| | TTCATGTTGAAACCCTAATTTTCAATGTGACTGTAGAAATCAGAACCTCTAAAGATGTAAT |
| | TGAGGTCATGGCAGGGGGTGTCCCTGATCCAGTAGGATTCATGATTTTGTAGGATCCAGAG |
| | ACCTCTCCTTTTTCTTCTCTCTCTCTAACCGCCTTCCCCTCCATGGAAAGGCTGTGTGAAG |
| | ACATGGTGAGAAGGTGGCACCTACAAACCAGGAATAAGGTCTTTAGCAGAAAACAAACTCT |
| | GCTGAACGTCGATCTGGGATATTCCAGGCACCAGAAATGTGAAAATTAAATTCTGTGGTTT |
| | AGCCATCCAGCCCCGTGTTACGGTGTGGCAGCGGAAGCTGACTCATCCATCTTCCCCACCC |
| | TCTGTGAGCAGATCAGCTTCAGGAGGCCCTCGTGGACATGGGGTCCCAGCTTTGCTCCTCT |
| | TCCTCCTGTTTTTCCAACTCTCTGGTGAAGAGGGAGGACTCAAGCTTCATCATCAGTTTCT |
| | GTGCATTAAACATGAATGTTTCCTTCAAGATGAAGTTTTTAGCCCATTTTGCTTTCTCAGA |
| | ATTTAATCAAACTGAATGAAGTATGTACTTACAATTAATATTTGGGGATGTTCATATTTGT |
| | TCCCCCCTTATATGACAGTTGGGATATTGTGTGGTGCTCATCTCCAGGCCCCTCCCTGTGT |
| | TCCAGGAGACAGGGTCACTGTCACCAGCAGAGCCAGTCAGGGAAATAACAGTGTCTAGCCT |
| | AGCTCCTTGTGAAATAAGGGCTGACACTGAGCTCCTGCTGGCTCCCACTGCTCCCTGGACA |
| | CGATGCCTGCCTGGGTCAAGGGGGTGAGTCTGGACAGACGTCACTCTGGCCATCAGTAGCC |
| | TAACTACCTTCATGACCTCCCACTGTGAACAGAGTCCCGGAAGCTGCTGGAGCCATGAAAG |
| | TTGGACAGAGAAATCCCACATCACTGCAGTCAGAGGGGGGCTATGAAAAGACCGTGGGGGG |
| | GTTTCATACTATGACCACCCAGCACTGAGCCATGGCTGCCACTCTGTCTGATGGGAGCCCC |
| | CAGGGGAAGATCCACTCACACTGTGCTCTAGAGGTCGTTTTTGTCACCATCTTTGTTCTAG |
| | CTGGTTTGGGCCAGTTTCTGTAGTGCATCCTGTTTTGTCCAGATCCTGTTCTGGTCAGCGT |
| | TGTCATGACCAGTGTTGTGATCAGTGCTCAGAATACAAGCCCTGATGATCTCCTACCTTAT |
| | ACTCACTGCCTTCTGTGAACCAGATGTTCTGATAAGGGTCCTGTTGGATCCTACTCGAATC |
| | AGTTGCCACACAGACCCTCACTGAGGGCTGAGGGCCACAGACATCTGAAGATAAACAGAGG |
| | TCCAGAGAATGATAGCCTGTGACTGTCCTCTGTAAAGACAGCTGCTCCCCAGATGGCTGAG |
| | GGCTGTCTTTGGCTGTGTCCTTCTTTCTGAATGATAATGAGGGATAGAGCAGGTCTCTAAG |
| | CAAAACCTCAGCAAGGCCTTCCTATGTGTGCTGCTCTAACCTGGACATAGGTGGCACTGGA |
| | TACACTTAGGGAAGTGAGGAAATTTATAGTCACAAGGAAGAGAGAGAAAGGAGGAGAGAGA |
| | GAGAAGAGCCTGTGATGTGTGTATAGTACCAACACTGATAGTATGTTCTTTAATGGTTTAT |
| | TGTTGAGTATGATGCTCAAATGCTCAGGTTCTATTCCATGCAGCCGATACATATACCTTAT |
| | ATAGGAAAAGGGCCTTTGCATATATAAATTAAGGATTATGAATTGGGCAGATTATCCTGTA |
| | TTGACCAGGTGGGCCCTAAATGGGCCTGTCTTTATTAAATGTCTGTCTATCTCTCTTATAA |
| | GGACATCACGATTGTCCTTATAAGATAGACATAGACAGACATTTTAACAAAGTCTGCAGAA |
| | GAGAAGGCAATTTGAAGACAGTGGCAGAGATTGAAGTGATGTGGACTCAAGCCAAAGAAAG |
| | TTGAAGCCACCAAAAGCTGGAAGAAGCCAAAAAGTAGACTCCCCGCTGGAGTCCCTGCAGA |
| | AGCTTTGATGACAGCCTGCTCTTGACCCCTGAAACTAGTGCTGGACTTATGGCCTCCAGAA |
| | CTATAGGAGGAGAATATATTTCTGTTGCTTTAAGCCACCAAAGTTTTAGTAATTTGTTATA |
| | GCAGCCCAAGAATTCTAATAAAAATGGGGCTTAGGATAAGTCCAGCCTAAAGGTAGTATGA |
| | TGATTTGCACTCTCCACCTTCATTTCTCTAATGTTACACATAATTGGTTAGAAGAAGATTT |
| | TTATGATGGAAACATTGTACAGGAAGCCACCCAGTATATACAGGGGCATCTGTTAGTTACA |
| | GAATAAATATTGACAGTTCTTAGTTGAAAATGACATCTGAGGCTGGGCTTGGTGGCTCACG |
| | CCTATAATCCCAGCACTTTGGGAGGCCAAGGCGGGTGGATCACAAGGTCAGGAGATGGAGA |
| | TCATCCTGGCTAACACAATGAAAAATCTGAAAAATACAAAATTTTTCAGAAATTCTGAAAA |
| | ATACAAAAAATTAGCCAGACATGGTGGCACGTGCCTGTAGTCCAAGCTACTCGGGAGGCTG |
| | AGACAGGAGAATCCCTTAAACTCGGGAGGTGGAGGTTGCAGTGAGCCGAGACCACACCACT |
| | GCACTCAGGCCTGGGCAAGAGAGCAAGACTCTGCCTCCAAAACAAACAAACAAACAAACAA |
| | ACAAAAAACCCACCAAACCAGGACTTTTTGAACTCAGCTCTGAACCAAGTGGACCTAATAG |
| | ACATCTACAGAACTCTCCATCCCAAATCAACAAAATATACATTCTTCTCAGCAGCACATCG |
| | CACTTATTCTAAAATTGACCACAAATGCCTTATGTAAATGACGAGTTGATGGGTACAGCAA |
| | ACCCATATGGCACATGTACACCTATGTAACCTGCAAGTTGTGCACATGTACCCCAGAACTT |
| | AAAGTGTAATAATACAAAAAAATGACATGTGACTAGTAGTATCTTATCTAGAATCTTCATT |
| | CTAAGATACTCAAGGACGCATAAAAGGGACCCTAAGTAGTCTTTTCATACATATATATGCA |
| | CATATATATGTATGAAAAGCAGTCTTTTCATCAACTAGAGAAACCCTCAGGACAGCCCTTA |
| | ATACCCTTGGTGATACATTTCAGATGAGTAAACTGTTATCAGAGCCCGTAGTTGAAACTAT |
| | TCAACAGAGATGGTTTGCCCAAAGATATGTGGTCAGCAATTGTCAGGGCTGAGCTTGGAAC |
| | CCAGGTCTGCATAACCTTAAATATGTTGCTTCCACATGGCCACGTTTGTTTCATATACGAT |
| | TGAATGGCCTTTAAATTCAAAGAAGAGACAAAGCCAGAAGAGTGGTGTGAAATTCTCAACA |
| | CAAGCTCCCTGCTACCTCTACACCTTACCGTGATTACTCCAATTATAAACTCAGGCCCTCA |
| | TGCAGTTTTGTCTACAAAGCAAAACTTCCTCAAAGTCTTTACAAATACTAAATGTCTTTCT |
| | TTCAGATTCGAGGGCAAGAGCACATCTTGCATTGCCCTGAACACTTTGCATCTTTTCTACC |
| | ATTCTCATCTTTCTGTCCCAGTCCTTCCTTCTCAAATGATGTCCTGTAAATCTGATTTCTC |
| | CCCCAATATGAAAACAAATGAACAAATATTCCCCTACTTTTCTCATATCCAGAGGATACAA |
| | GAGTTAATCACATATCCAGAGAGTACGAGAGTTAATCAAGGGATTTATGCAAGAGTGTTTA |
| | CACATAACAAGGATTCTGGTGCTAGCCATCTTCACAGTGAAATTTTCTGTGTGTCTTGCTA |
| | AAATTGACACTAAAAAATGACAAGATAAAAATATTTGGAAGAACAGAGGGCAACCATGCCC |
| | TTAAGGAGGTAAAAGACACCCCTGCCCCTTGTGTTAGTTTCCTACTCCTGCTGTAACAAGT |
| | TATCAGAATCTTACTAGTTTCATACAACACAAATTTATTATCATACAGTTCTGGCAGTGAG |
| | AAGTCTCACTGATTTATAATCAAGGTATCCGTAGTTCTATATTCCTTCTGGAAGTTCCAGG |
| | GGAGAGAATCTGATTCTCAGCTTTTCATCTTCAAAGATAGCCCCATGTTCCGGGCTGCCTG |
| | GCCCCTTTCTCCATCACTGAAGCATCCCTGTCCATTGTCCCTATTCCTCTCTGACTGTTAC |
| | CCCCACTCCTCCCTATTATAAAGACCCTTCTGATGACGCTGTCTTTCCTAGATAATTCAGC |
| | TGTTTCCTAAATTTTCTGAATATCCCTATGCATGAAAAAAAAAGAATTGGCAAGTATTCAG |
| | ACTATACTTTCCAAGAATGAGGGTTTGTCCACTGTTTTAGGTTGGATCTTTCAGGGACAAT |
| | GATGCCCATGCAGGCAGCATATTTATAATGCACAGTAAACACTAGGAGGAAACAAGGCAGT |
| | GAGAGAGGAAAGAGAGCAGCGATACCGAAAATGTCCTCAGCGAGAAGCTACCACAGAGGAT |
| | GAATGGAGATCAAGCCCACGTGGAAACATGGGAAAATGTCTCAGTATTTTTCCACCTAAGA |
| | AGGGAGGGAGATGGGGTATGTATACACCTCCCTGTCCTCACTGATTGAGGGCTTTCCGAGA |
| | GGATGCTCATTCCAGGTGCTGTGATAGGCCATGTGTACAGGCAGGGCTGCCTTCTCCAGCT |
| | TCAGATAGAGCAGTGAGGAAAAGATATGGCCATGGGGGGTCAGCAGAAGTACAGCAAAGGG |
| | AAAAGGGAAAGGGTAGCAAGAGTGACAACTATATTCACCCCCCCCACACACACACACACAC |
| | ACACGAAATTGTGTATTGCAATCCAGAACTGCTTCTCTCTGAACCTAAATCTTAGCAAGCA |
| | GTTTACCAGTAACTGCCCTTGAAATTCAGGCCCCTGGAAAGGAGCAGGGGGTTGTGTACAG |
| | GCTATACCACAGCAGTCTGCCCACCCTTAGTGATGCATGAGTAATGCTCCCTGGACTCCCC |
| | AGGTTCTAGTCTTCTCATGTCGATGTAGTTGATTCCACTTCCCTTGCTGCACAACCAGGCT |
| | GGGATGCCTGGGCAGAGGCAGACATGTGAGGTATAGGGGTTCAAATCTGTTTCCAAGTTTT |
| | ATCCAGCTTCAAAGCATTTCTCCGTGTACATGAGCGGTGGCTTGACAGGAGATGGAGACTC |
| | TCTTTCCTGGATGTGAGGCAAGGAGGCAGGCGTCTGAGTCAGGATGATGTCCCTACTCACT |
| | GCTAAAGAGAAAAGTGGCTTTGATGGTGCAGGGCAGGGAAATGCACTGAGTGGTCGCCACC |
| | CTCACAGAAGAGAAAGTGTTCACTGACCTGGCCTTTCCCCAGGGCCTCTCCCTCCCATTGC |
| | TTTCCAGAAAGCCATGATTTTTGAGAGCCACACCTGAACACTCACAAACATTATGGTGGGA |
| | AAAGCAGATCAGAGCATTAGGCAAGTTGCATTACCTTGGCCTTCTTCCTTTGGAGACAATT |
| | GATGTGGGGTTCTAGATTGACCCAGAGTTTCAAGTTTATCCTGATTCAGGCTTCAACAGCT |
| | GGAGGAAGAAACAGAGATGTTTTTTGAAGTAAACAGATCTAGCATTACTAATCAACCCTTC |
| | ATACTGATGACCTATGGGAAATAATACCCAAGGGCAGAAAAATGGGCAGAATAAGGGGAGC |
| | CCCAAACCAAGACGAAGCTGCTGCCCATTGAGACCCTGGGTATTACAGAGACCTATAGCTC |
| | TGGATAATGGAAGATCTATGAGTGGCACAGGCGCTGAGGAATCACAGCATCATTATCGTGC |
| | ATCTGCAGGGAATTGCTTGTAAATATACTGGTAATTACAAATGTTTAAGGTCACTACAAAT |
| | ACTTTGGAGTGTATTAAATATGCTTCTGATAAAGACTGTTTTTCTCACATGAAACAATGGG |
| | AACCATGTGACAATCACAGAGGTGTTGTTACTATAGCAAAAGGGATTGTTACTCTCCACAT |
| | CCCTTTAAGTAACTTGAAGGCCTGATAGACCCACCCTCTAAGACTTCATTAGACATTCCCT |
| | ACGAATGGTTATACTCTCCTGTATACTCCCAATACAACTCTAAAATATATTATTCCATATA |
| | GTCCTTAGGTTTGTATTAAAGTTTGACTTTTTTCCTTCAAAATATCTCTTGTCACAACAGC |
| | GGCTCTAGAGAGAAATACATTCCCTCCAGGCAAATCTATGCTGCGCTGGTCTGACCTGGGA |
| | CCCTGGGGACATTGCCCCTGTGCTGAGTTACTAAGATGAGCCAGCCCTGCAGCTGTGCTCA |
| | GCCTGCCCCATGCCCTGCTGATTGATTTGCATGTTCCAGAGCACAGCCCCCTGCCCTGAAG |
| | ACTTTTTTATGGGCTGGTCGCACCCTGTGCAGGAGTCAGTCTCAGTCAGGACACAGCATGG |
| | ACATGAGGGTCCCCGCTCAGCTCCTGGGGCTCCTGCTACTCTGGCTCCGAGGTAAGGATGG |
| | AGAACACTAGGAATTTACTCAGCCAGTGTGCTCAGTACTGACTGGAACTTCAGGGAAGTTC |
| | TCTGATAACATGATTAATAGTAAGAATATTTGTTTTTATGTTTCCAATCTCAGGTGCCAGA |
| | TGCGACATCCAGATGACGCAGAGCCCGTCCTCGTTGAGCGCCTCCGTCGGCGACCGCGTGA |
| | CTATCACTTGTCGGGCGTCCCAGTCTATTAGCTCCTACCTGAACTGGTATCAGCAGAAGCC |
| | AGGAAAGGCTCCCAAGCTCCTTATCTACGCCGCATCATCGCTGCAATCCGGAGTGCCGTCC |
| | CGGTTTTCGGGCTCGGGTTCCGGCACCGACTTCACCCTGACCATTAGCTCCCTGCAACCCG |
| | AAGATTTCGCCACCTACTACTGCCAGCAGTCCTACTCAACTCCTTACACCTTCGGACAAGG |
| | GACCAAACTCGAGATCAAGCGTAAGTACTTTTTTCCACTGATTCTTCACTGTTGCTAATTA |
| | GTTTACTTTGTGTTCCTTTGTGTGGATTTTCATTAGTCGGATGCCAGGGATCTAACAAACT |
| | TCATTCCCAGGTTAGGTACAGAGGAGGGGAAATTGTTCCACAGGACGCTAGCTTGTGGCTA |
| | ATTTTTAAGATTTCTAAATCAAAATAACTTCATTGGGGGAAAGAGGCTTGCTGAGCTTTCA |
| | GGGACACAGTGTTACAAGTCATAACATAAACCAAAACACCCTGACCCCTCTGCCTGGCATA |
| | GACCTTCAGACACAGAGCCCCTGAACAAGGGCACCCCAACACCTCATCATATACTGAGGTC |
| | AGGGGCTCCCCAGGTGGACACCAGGACTCTGACCCCCTGCCCCTCATCCACCCCGCAGGTT |
| | CTCAACCCTCACCCCCCACCACGGGAGACTAGAGCTGCAGGATCCCAGGGGAGGGGTCTCT |
| | CCTCCCACCCCAAGGCATCAAGCCCTTCTCCCTGCACTCAATAAACCCTCAATAAATATTC |
| | TCATTGTCAATCAGAAATCTTGTTTTATCTCATTTTTTCTTTTCTCACATATAATTCCTAG |
| | CCTTTCCTGGGTTCTCAATTTGTGGTGGGGTTTATGTTATGACTTGTAACACTGTGTTTCG |
| | CTGTTAATATCACTAACCTGACCGATGCAGAGAAAATCTTGCAATTGAGATGCCTCACTTA |
| | ACTGGCTAGCGCTTGGCTGTTCCTTAAGATGAACTAATTTTCTATCCCTTACTCATCTGAC |
| | TTTTTGAAAGAATCTGGTACTCTTTGGAATTGACCTGAGCTAATATCTCAAACACAAAAAC |
| | GCTCCAAATTTAAAACCTTATAAGAAAAAGCATTAGGAAAGTGCACTTACGCTTAATCTCG |
| | ACCTTGGTTCCACCCCCAAAGGTAAGGGGAGTGGAGTAGTACTGTTGGCAGTAGTACACTG |
| | CGAAGTCCTCGGCCTGCAGGCTGCTGATCGTCAAAGTGAAGTCAGTGCCGGAGCCGCTCCC |
| | CGAGAACCGATCCGGCACTCCGGATTCGCGGGTCGAGGCCCAGTAGATGAGCAGCTTAGGC |
| | GGCTGTCCTGGCTTTTGCTGATACCAGGCGAGGTAATTCTTGTTGTTTGAGGAGTACAGCA |
| | CGGACTGCGAGCTTTTGCAGTTGATGGTAGCTCTCTCTCCCAGGGACACGGCCAGGGAGTC |
| | GGGTGACTGGGTCATCACAATTTCGCCGTACGCACCTGTAGTCAGTAATGGACAATGATTA |
| | TACATAAACTTATACACGCTGTCTCAGATATAATTGAAATATGTCATTTAAATTTGCTTTC |
| | TAGTAGTATTATAAGTCAGCAGATAATTGGATCCATATTGGAAATAGTCATTATTTCCAAC |
| | TATATCTTAATTTTTTTTATTTCTACACAGATATTACTCTGAAAAGACTGTGGCACTTTTT |
| | AATTCCTCACCAGAGATCCAGAGCAACAGAGAAATGAAGACCTGGGTCTGCAACACCATCT |
| | TGCTGCCCCTGCCTGCCTGTTGTAGCTCAGTTCACAATGCAAACGGCCCGTTTATAAAATC |
| | CAAACAGCTGGTCGTGGCCTGGAGGGGCCTATGCAAAAACAACCAGTGAGTACAAAAGCAA |
| | ATTTCATGAGCAGTGGGTTGTGAAAGTACCCAATGTAAATCAAGAGCCAAAAATATCATCA |
| | CAGAATGTGTAATTGTATGAATAGAAAAGACAGAAGTCAACTTAGAAGCTCTAAAACTACA |
| | AACTGCTGAAAGATCTAATGACTAGGACAGCCTAGTAATTTTCATAGGGGCATAAATGTGA |
| | AACGCCTTGTGCATCGTAGAAGAAAGCAGAAGAGAAAGCATTCCCAATTTCTTAACTGCCT |
| | TTTACCTATATTAATCAGTAATATACTGGCTTTTACCTCTGTTAATCATAATAAACAAATT |
| | CTCAATAAATTTTATCGATACTCTTCAATGCCTGCTCAGCAACATTTTCCGAAGGCAGCTC |
| | AAGATATTAAATAACTCATAAGGGCCAACCTCCTATTGCAGCATTCTTTGGGATTTAACCA |
| | GTTTCCCAAGACTCTTTTCACAATGTTAAGATGTTAGAAATAGATCCAAAACTAGGTGATA |
| | TATCCCCTAGTAAAACTGTGAGGTCAAACTTGTCTGGCTAATGCTTCCATTTAAAAATTTC |
| | TCTTTCTTGATCCTTCATTGTATGTACACAATAAATCAGGGGAAAACTTTAACTGAGTGAA |
| | TCAAAGTATTCTCATTATTATAATAGGAGCTTCACACACACACAAAAAAATCAATTCTATT |
| | ACTCTCAGCCTCAGTTCCTAAAGCCAAGTTAAAGTCCTGTTCTAAGATCATTGTTGCATGA |
| | CCATATGTATTCCAGGTCTAATCTAAACTGTGGATAAATCCCAGCAGGACATTAGAGATTT |
| | TTGTGAGAGTAAGCATATAGGATTCAGGGTTTATGAGCTTTAGATTTTTCTTGTCAAAATG |
| | AATGAGAGTTGCCATATCTAAAAATTATTCCCAGATAAATAAAATTCACTACCTAGAATTA |
| | ATTTATGCATATAAGTAGAAATGCTATCTCCCTTTTTACCATCCAAAGTGGAAAGCCTCAT |
| | GGAACTAGAAATTAATATTAGAAAAATCAGTTAATAAAAGTATGTCATTTCATCAATTCAA |
| | TAAGTTATAATAGCAAAAAACCATAATAAATTATCACTTAAATGTCAATACATTTATAAAC |
| | TATGGTACATAAATAGGATATTGAATAGCCATTGATGCTCCTGATGAAAATTAGCAGGCAG |
| | TGATAAATGATAAATATGAAGCACATGTCAATAAATAAAATAAGTTTTATGTAATTTAGGA |
| | GAAAATGGTGATAATGACACAAAATGTGAATTATGGATGCATCTATAAAATTCTTTGTACA |
| | TTTGTGAATTGTAAATATTTATCTTAGAGACATTATTACTTTGTATATGTTCCATTTGCTC |
| | ACCTATATGTCCCAGTCTCCTTACAAATGCTATGGCCAAAGAAATAGGCATACATACATCC |
| | TTTGCAGGCTGAGGCAGGAAAAAGATCTTACGGAATTTTCCAGTCTATCCTTTATCTGTAT |
| | AAGCAACTTAAGAGGCCATGTGCTCCAAATGGTGCAAATACAAGGGTTTTTGTTCAGCAAG |
| | ACAATGGAGAGCTCTCACTGTGAGTAATTTTTCACTATTGTCTTCTGAAATTTGGGTCTGA |
| | TGGCCAGTATTGACTTTTAGAGGCTTAAATAGGAGTTTGGTAAAGATTGGTAAATGAGGGC |
| | ATTTAAGATTTGCCATGGGTTGCAAAAGTTAAACTCAGCTTCAAAAATGGATTTGGAGAAA |
| | AAAAGATTAAATTGCTCTAAACTGAATGACACAAAGTAAAAAAAAAAAGTGTAACTAAAAA |
| | GGAACCCTTGTATTTCTAAGGAGCAAAAGTAAATTTATTTTTGTTCACTCTTGCCAAATAT |
| | TGTATTGGTTGTTGCTGATTATGCATGATACAGAAAAGTGGAAAAATACATTTTTTAGTCT |
| | TTCTCCCTTTTGTTTGATAAATTATTTTGTCAGACAACAATAAAAATCAATAGCACGCCCT |
| | AAGAAAAATCAGGGAAAAGTGAAGTGTACCTATTTGCTATGTAGAAGAGGCAGCTTACTTG |
| | AAAATCAGCAGCAATGTTGTTTTTAGAGTCTGTAATAAGTAATAAACTCAAAAAGACACAT |
| | TCTATAGGAATAAGGGCTTCACAGATAGAGCTCATTTTTTAAAAATCCAATTTGTACATTA |
| | GACTAAACGTGAAATTATCTCTTATTGTAATGGTGGAAAGGTGGTTATTCCCAAAAGCTCA |
| | ATCTCAAAGAAATGTGTTTAAATGAAAAAAAGTAAATAATTGCATTTTTTAATGACCGTGG |
| | GTCTGTGAAAAAAATAGGAAATATTTTAAAGAGTATGTTCTTTCATTATCCTCTGTTATTA |
| | CTTGTCTACATTTTTATTCTGCCAAGAAGGCCGTGGCACCGCGAGCTGTAGACAGAGCCGC |
| | GGTCTTTCTCGATTGAGTGGCTTTGGTGGCCATGCCACCGCGCTCTTGGGGCAGCCGCCTT |
| | GCCGCTAGTGGCCGTGGCCACCCTGTGTCTGCCCGATTGATGCTGCCGTAGCCAGCTTTCC |
| | TGATGCACAGTGATACAAATAATGCCACTAAGGGAAAGAGAACAGAAACGTAATGGGCGCT |
| | GAGCTGGGAAAACCAGGGAGAAGACTGATTTATTAGAGATTTCAGAAATAAAATTCACATT |
| | CATTATGATATCTCATTAGTGAAAATTTCCATTAGGGGATTGTAAATAATTTAAAGCTTTT |
| | TTTTTTTTCAGTGCTATTTAATTATTTCAATATCCTCTCATCAAATGTATTTAAATAACAA |
| | AAGCTCAACCAAAAAGAAAGAAATATGTAATTCTTTCAGAGTAAAAATCACACCCATGACC |
| | TGGCCACTGAGGGCTTGATCAATTCACTTTGAATTTGGCATTAAATACCATTAAGGTATAT |
| | TAACTGATTTTAAAATAAGATATATTCGTGACCATGTTTTTAACTTTCAAAAATGTAGCTG |
| | CCAGTGTGTGATTTTATTTCAGTTGTACAAAATATCTAAACCTATAGCAATGTGATTAATA |
| | AAAACTTAAACATATTTTCCAGTACCTTAATTCTGTGATAGGAAAATTTTAATCTGAGTAT |
| | TTTAATTTCATAATCTCTAAAATAGTTTAATGATTTGTCATTGTGTTGCTGTCGTTTACCC |
| | CAGCTGATCTCAAAAGTGATATTTAAGGAGATTATTTTGGTCTGCAACAACTTGATAGGAC |
| | TATTTTAGGGCCTTTTTAAAGCTCTATTAAAACTAACTTACAACGATTCAAAACTGTTTTA |
| | AACTATTTCAAAATGATTTTAGAGCCTTTTGAAAACTCTTTTAAACACTTTTTAAACTCTA |
| | TTAAAACTAATAAGATAACTTGAAATAATTTTCATGTCAAATACATTAACTGTTTAATGTT |
| | TAAATGCCAGATGAAAAATGTAAAGCTATCAAGAATTCACCCAGATAGGAGTATCTTCATA |
| | GCATGTTTTTCCCTGCTTATTTTCCAGTGATCACATTATTTTGCTACCATGGTTATTTTAT |
| | ACAATTATCTGAAAAAAATTAGTTATGAAGATTAAAAGAGAAGAAAATATTAAACATAAGA |
| | GATTCAGTCTTTCATGTTGAACTGCTTGGTTAACAGTGAAGTTAGTTTTAAAAAAAAAAAA |
| | AACTATTTCTGTTATCAGCTGACTTCTCCCTATCTGTTGACTTCTCCCAGCAAAAGATTCT |
| | TATTTTACATTTTAACTACTGCTCTCCCACCCAACGGGTGGAATCCCCCAGAGGGGGATTT |
| | CCAAGAGGCCACCTGGCAGTTGCTGAGGGTCAGAAGTGAAGCTAGCCACTTCCTCTTAGGC |
| | AGGTGGCCAAGATTACAGTTGACCTCTCCTGGTATGGCTGAAAATTGCTGCATATGGTTAC |
| | AGGCCTTGAGGCCTTTGGGAGGGCTTAGAGAGTTGCTGGAACAGTCAGAAGGTGGAGGGGC |
| | TGACACCACCCAGGCGCAGAGGCAGGGCTCAGGGCCTGCTCTGCAGGGAGGTTTTAGCCCA |
| | GCCCAGCCAAAGTAACCCCCGGGAGCCTGTTATCCCAGCACAGTCCTGGAAGAGGCACAGG |
| | GGAAATAAAAGCGGACGGAGGCTTTCCTTGACTCAGCCGCTGCCTGGTCTTCTTCAGACCT |
| | GTTCTGAATTCTAAACTCTGAGGGGGTCGGATGACGTGGCCATTCTTTGCCTAAAGCATTG |
| | AGTTTACTGCAAGGTCAGAAAAGCATGCAAAGCCCTCAGAATGGCTGCAAAGAGCTCCAAC |
| | AAAACAATTTAGAACTTTATTAAGGAATAGGGGGAAGCTAGGAAGAAACTCAAAACATCAA |
| | GATTTTAAATACGCTTCTTGGTCTCCTTGCTATAATTATCTGGGATAAGCATGCTGTTTTC |
| | TGTCTGTCCCTAACATGCCCTGTGATTATCCGCAAACAACACACCCAAGGGCAGAACTTTG |
| | TTACTTAAACACCATCCTGTTTGCTTCTTTCCTCAGGAACTGTGGCTGCACCATCTGTCTT |
| | CATCTTCCCGCCATCTGATGAGCAGTTGAAATCTGGAACTGCCTCTGTTGTGTGCCTGCTG |
| | AATAACTTCTATCCCAGAGAGGCCAAAGTACAGTGGAAGGTGGATAACGCCCTCCAATCGG |
| | GTAACTCCCAGGAGAGTGTCACAGAGCAGGACAGCAAGGACAGCACCTACAGCCTCAGCAG |
| | CACCCTGACGCTGAGCAAAGCAGACTACGAGAAACACAAAGTCTACGCCTGCGAAGTCACC |
| | CATCAGGGCCTGAGCTCGCCCGTCACAAAGAGCTTCAACAGGGGAGAGTGTTAGAGGGAGA |
| | AGTGCCCCCACCTGCTCCTCAGTTCCAGCCTGACCCCCTCCCATCCTTTGGCCTCTGACCC |
| | TTTTTCCACAGGGGACCTACCCCTATTGCGGTCCTCCAGCTCATCTTTCACCTCACCCCCC |
| | TCCTCCTCCTTGGCTTTAATTATGCTAATGTTGGAGGAGAATGAATAAATAAAGTGAATCT |
| | TTGCACCTGTGGTTTCTCTCTTTCCTCATTTAATAATTATTATCTGTTGTTTTACCAACTA |
| | CTCAATTTCTCTTATAAGGGACTAAATATGTAGTCATCCTAAGGCGCATAACCATTTATAA |
| | AAATCATCCTTCATTCTATTTTACCCTATCATCCTCTGCAAGACAGTCCTCCCTCAAACCC |
| | ACAAGCCTTCTGTCCTCACAGTCCCCTGGGCCATGGTAGGAGAGACTTGCTTCCTTGTTTT |
| | CCCCTCCTCAGCAAGCCCTCATAGTCCTTTTTAAGGGTGACAGGTCTTACAGTCATATATC |
| | CTTTGATTCAATTCCCTGAGAATCAACCAAAGCAAATTTTTCAAAAGAAGAAACCTGCTAT |
| | AAAGAGAATCATTCATTGCAACATGATATAAAATAACAACACAATAAAAGCAATTAAATAA |
| | ACAAACAATAGGGAAATGTTTAAGTTCATCATGGTACTTAGACTTAATGGAATGTCATGCC |
| | TTATTTACATTTTTAAACAGGTACTGAGGGACTCCTGTCTGCCAAGGGCCGTATTGAGTAC |
| | TTTCCACAACCTAATTTAATCCACACTATACTGTGAGATTAAAAACATTCATTAAAATGTT |
| | GCAAAGGTTCTATAAAGCTGAGAGACAAATATATTCTATAACTCAGCAATCCCACTTCTAG |
| | ATGACTGAGTGTCCCCACCCACCAAAAAACTATGCAAGAATGTTCAAAGCAGCTTTATTTA |
| | CAAAAGCCAAAAATTGGAAATAGCCCGATTGTCCAACAATAGAATGAGTTATTAAACTGTG |
| | GTATGTTTATACATTAGAATACCCAATGAGGAGAATTAACAAGCTACAACTATACCTACTC |
| | ACACAGATGAATCTCATAAAAATAATGTTACATAAGAGAAACTCAATGCAAAAGATATGTT |
| | CTGTATGTTTTCATCCATATAAAGTTCAAAACCAGGTAAAAATAAAGTTAGAAATTTGGAT |
| | GGAAATTACTCTTAGCTGGGGGTGGGCGAGTTAGTGCCTGGGAGAAGACAAGAAGGGGCTT |
| | CTGGGGTCTTGGTAATGTTCTGTTCCTCGTGTGGGGTTGTGCAGTTATGATCTGTGCACTG |
| | TTCTGTATACACATTATGCTTCAAAATAACTTCACATAAAGAACATCTTATACCCAGTTAA |
| | TAGATAGAAGAGGAATAAGTAATAGGTCAAGACCACGCAGCTGGTAAGTGGGGGGGCCTGG |
| | GATCAAATAGCTACCTGCCTAATCCTGCCCTCTTGAGCCCTGAATGAGTCTGCCTTCCAGG |
| | GCTCAAGGTGCTCAACAAAACAACAGGCCTGCTATTTTCCTGGCATCTGTGCCCTGTTTGG |
| | CTAGCTAGGAGCACACATACATAGAAATTAAACGCGTATAACTTCGTATAAAGTATCCTAT |
| | ACGAAGTTATGCGGCCGC |
| | (full length binary LC construct) |
| 6 | CSSYTSSSTLVVFGGGTKLIVL |
| | (Lambda VL2-14/JL2 Consensus) |
| 7 | CQSYDSSLSGYVFGTGTKVTVLG |
| | (Lambda VL1-40/JL1 Consensus |

1. A transgene construct comprising:
(a) a first immunoglobulin light chain variable cassette (VL1) and a second immunoglobulin light chain variable cassette (VL2), wherein each of VL1 and VL2 comprise a promoter, a light chain V region, a light chain J region and a splice donor site;
(b) a stop cassette (SC) comprising a splice acceptor site and a polyadenylation signal; and
(c) a first recombination signal sequence (RSS) 12 mer (RSS1), a second recombination signal sequence 12 mer (RSS2) and an RSS 23mer (RSS3);
wherein the transgene construct comprises, from 5′ to 3′
VL1-RSS1-SC-RSS2-VL2-RSS3
and wherein VL2 is in antisense orientation relative to VL1.

2. The transgene construct of claim 1, wherein in a B cell carrying the transgene construct, prior to RAG-mediated recombination VL1 and VL2 are inactive and after RAG-mediated recombination either VL1 or VL2 is active.

3. The transgene construct of claim 1, wherein the light chain V regions and J regions are human kappa sequences.

4. The transgene construct of claim 3, wherein VL1 or VL2 comprises a Vκ 1-39 region.

5. The transgene construct of claim 3, wherein VL1 or VL2 comprises a Jκ JK2 region.

6. The transgene construct of claim 3, wherein VL1 or VL2 comprises a Vκ 1-39 region and a Jκ JK2 region.

7. The transgene construct of claim 3, wherein VL1 or VL2 comprises a Vκ 4-1 region.

8. The transgene construct of claim 3, wherein VL1 or VL2 comprises a Jκ JK4 region.

9. The transgene construct of claim 3, wherein VL1 or VL2 comprises a Vκ 4-1 region and a Jκ JK4 region.

10. The transgene construct of claim 3, wherein VL1 comprises a Vκ 1-39 region and VL2 comprises a Vκ 4-1 region.

11. The transgene construct of claim 3, wherein VL1 comprises a Vκ 1-39 region and a Jκ JK2 region and VL2 comprises a Vκ 4-1 region and Jκ JK4 region.

12. The transgene construct of claim 3, wherein VL1 comprises a Vκ 4-1 region and VL2 comprises a VK 1-39 region.

13. The transgene construct of claim 3, wherein VL1 comprises a Vκ 4-1 region and a Jκ JK4 region and VL2 comprises a Vκ 1-39 region and a Jκ JK2 region.

14. The transgene construct of claim 1, which further comprises a light chain constant region downstream of RSS3.

15. The transgene construct of claim 14, wherein the light chain constant region is a human kappa constant region.

16. The transgene construct of claim 14, which further comprises an enhancer downstream of RSS3 and upstream of the light chain constant region.

17. The transgene construct of claim 16, wherein the enhancer comprises an intronic human kappa enhancer (mEKi).

18. The transgene construct of claim 1, wherein VL1 or VL2 comprises a CDR3 comprising the sequence shown in SEQ ID NO: 1.

19. The transgene construct of claim 1, wherein VL1 or VL2 comprises a CDR3 comprising the sequence shown in SEQ ID NO: 2.

20. The transgene construct of claim 1, wherein VL1 or VL2 comprises the sequence shown in SEQ ID NO: 3.

21. The transgene construct of claim 1, wherein VL1 or VL2 comprises the sequence shown in SEQ ID NO: 4.

22. The transgene construct of claim 1, which comprises the sequence shown in SEQ ID NO: 5.

23. A transgenic animal comprising the transgene construct of claim 1.

24. The transgenic animal of claim 23, which is a mouse.

25. The transgenic mouse of claim 24, which further comprises a transgene construct encoding an immunoglobulin heavy chain such that the mouse expresses antibodies comprising the heavy chain paired with a light chain comprising the light chain V region of either VL1 or VL2.

26. A method of generating antibodies to an antigen of interest, the method comprising administering the antigen of interest to the transgenic mouse of claim 25, such that antibodies that bind to the antigen of interest are generated.

27. (canceled)

28. The transgene construct of claim 1, wherein the light chain V regions and J regions are human lambda sequences.

29-42. (canceled)

43. A transgenic animal comprising the transgene construct of claim 28.

44. The transgenic animal of claim 43, which is a mouse.

45. The transgenic mouse of claim 44, which further comprises a transgene construct encoding an immunoglobulin heavy chain such that the mouse expresses antibodies comprising the heavy chain paired with a light chain comprising the light chain V region of either VL1 or VL2.

46. A method of generating antibodies to an antigen of interest, the method comprising administering the antigen of interest to the transgenic mouse of claim 45, such that antibodies that bind to the antigen of interest are generated.

47. (canceled)


SEQUENCE LISTING (5 sequences)

SEQ ID NO: 1 - protein, 11 aa, synthetic construct [sequence omitted]

SEQ ID NO: 2 - protein, 11 aa, synthetic construct [sequence omitted]

SEQ ID NO: 3 - protein, 236 aa, synthetic construct
MDMRVPAQLLGLLLLWLRGARCDIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 240 aa, synthetic construct
MVLQTQVFISLLLWISGAYGEIVMTQSPDSLAVSLGERATINCKSSQSVLYSSNNKNYLAWYQQKPGQPPKLLIYWASTRESGVPDRFSGSGSGTDFTLTISSLQAEDFAVYYCQQYYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 35459 bp, synthetic construct
  source 1..35459: mol_type = other DNA
gcggccgcataacttcgtatagcatacattatacgaagttatggcgcgccattcgcgactcgagcgcgccgcacacaaaaaccaacacacagatcatgaaaataaagctcttttattggtaccgaattcgccagggagctctcagacgtcgcttggtcggtctttattcgaaccccagagtcccgctcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtttagttcctcaccttgtcgtattatactatgccgatatactatgccgatgattaattgtcaacacgtgctgctgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtggaaggtggcgcgaaggggccaccaaagaacggagccggttggcgcctaccggtggatgtggaatgtgtgcgagccagaggccacttgtgtagcgccaagtgcccagcggggctgctaaagcgcatgctccagactgccttgggaaaagcgcctcccctacccggtagcacgactcgtacgtacgtacgaagttcctattctctagaaagtataggaacttccactgtggcaggtcagcacccctgtttatgttcctgtcacctgccacagccttgactctcataaccaacaggaaaatgaggaggttctagggccctgtgagtaaaaaactgggatgatagggaaaggagaatggaatctcatctgaatcctccttccttgcctacatttgtttaaatttattgagcaaaagggccagactactgattatttctggcaaaacatgttgagtacattttagggtttaacagttttgggtacctttcaaagaaaatatttggttatatgtaaaattggtattttcccactttttaaattcctacttctcctgtttgccattcttctccactccatgagacagtagagacaggattattcactaattctcctctggcggagctggctgaggacaatcagtaagatcttggttgtgagtgtcaaatagattttgtaatttcatagcagacgcaagttcctaatactaaaactctttgattacaattacctcttgctgatagaaaaagggagttcttgaaattttgaaagttggttttaaaaataaaatgcatacactggaagatgcagtatactaaagatgtagtattttccagggatcactgagaaaatacaggatgaggtaaacagttttattttccaaagttcagaatttgagattgggcagactgcaggaatgggagcatgaagagaacataagagagatcagctattgttcaattacactgcccttgattatggtgggtggggatgtggctggtggtggtgatggcagttgatgatgtggatccaaaaaggggccaaatgtgttacttgtgaagaaccacagagttgaaagcactgctgcatggcttcctgggtggagccaagtcttcgcactttccaaactttctctctcctttattactcacatgaaactgccctctctagtattatggtggaaaaagcattctgcaccagctattttcatgggagtatggccaaggataattatttttactacttatggatttttaaaatccaggacagatataaatcctaataccaaaatagtttgatttgcctcagttgccttttgctgactgaaaatagagttcttccaattccaaaagtgggctttgaaaatagacaaaataattcagatggagaacataaagtttacataatgtaccacaggaacaatgcagaattatatgagatttttatttccttctcaaattcttagaattttaaaaatattttactgacatagcattttaaagagaaaatttttagtgctatgttgtcataagaaaatgattcaaagaatgaataaatgcattatttttatatgatactattcctgaaaataaatctgaatcctctattttagttgttaatgcatagaaaaattatgctcttaatatattccattgacaatagtgctctaaatttatatgcttccttaatttgagggctacagtctgataaatatctatctacattttgtcatgcaacttttaaacctaacagaaatgctttcgttaaaaaagcaacagtgctttctctaccataatgctagagagggcagtttcgtgtgagtggtaaaggagaaagtttggaaagtgggaagacagcaattttgcaatatgaccaagtgttatcatgcaaaaaactatctccaaaccagtcatttctaacatgtatttcacttgtgttaccactaatctgcatccatgatctgatcattctgccaaataaatttacacgaacccgagtaatgagtgatgtctgtcacatgtgataacacggtgtagagacacaaaagactgaatcaacagaacaagatcctgatacaggctaagggtttaaataatacttgagatgatttatttcaagataacagattccataggaagtgcaattaaaattccaatgtgtcatttctgttaggtttaaaagttgcgtggcaaaatgtacatagatatttattagaccggagctctcaaactaagaaatatgagacactcaatatgttattgaaaaatactaattaatgcatcattgaccctatattttattacataatgcagtaaaagaaaataaaatgtaagtagcaagagcacgaggtaagcaatgccatcgtcacctttagccctcctgggattgacagcacctagtcaccttgagtttctgcttttctgcgagacaggagataaaatgaaaacccattcaaggtagttagttatattttggagaaagcaaacaaagataccacctacatgatgctgatatcccaaaggcatatattctcaagtcaaaatagtgaaaagtatatgatccccaaaactgaaaggggagagatacaaggagaatcagagcatgatcaaatttaattacaaagaagcctcataatatggtgaactaaatgtaacaaggtttctttgtctgttgccatggcagtgcagaggcaggcaggtggccttggtggtgtaggtggctctgctccgtgaggtcactccggtgggcaggaggcacgaacaccctgagaacacagccttcctccttcctcacagtcattgcctgcccagccatcctcagcagcatgaggtgggacagagtggagagaaagctgttttcttctaaagactaaaaacaaaaacagaaaaaaaaaaaacaaacctggagctttccatcagtgacaaatgtacttttgactcaatcacacatttgagaagttttccagtgagtggatctgcagataaacccacattcgttgtttgtttgttttaatctgaaaatgtgtttacatgattcttgaaaatattttttgactgtaaacttatatttgtagtagcctatttgaagttatcatttactatttcataattgctactaaaaagttattgttaaaaaaatctgtgactctaactgttcttgtttgaaaggaatacgtctttttaagacactcagactccttttaagctcctcatttggccctcctttgtttctgattcaatgtattgtttaatttgttatttatgactaattaatctattaattaattttcacaatcacagaatcaaatgtcctataagttgctatgtcaaagacctgcctgaagatggcacacatgctccacaatgaacaaaggacagcaccatggtctcaggaacactggggaagtaggagtgctggtgtcccattatcaacagggagccctagagttagagtcaaatcttccctgtgacctgggcacctgggaggagccacccgtgtgctgagctgtgggaacctgccccatgccctgagactggaagcacggccttggctgtgtcccacctgctatggactgaattgtgccctcagattcatgttgaaaccctaattttcaatgtgactgtagaaatcagaacctctaaagatgtaattgaggtcatggcagggggtgtccctgatccagtaggattcatgattttgtaggatccagagacctctcctttttcttctctctctctaaccgccttcccctccatggaaaggctgtgtgaagacatggtgagaaggtggcacctacaaaccaggaataaggtctttagcagaaaacaaactctgctgaacgtcgatctgggatattccaggcaccagaaatgtgaaaattaaattctgtggtttagccatccagccccgtgttacggtgtggcagcggaagctgactcatccatcttccccaccctctgtgagcagatcagcttcaggaggccctcgtggacatggggtcccagctttgctcctcttcctcctgtttttccaactctctggtgaagagggaggactcaagcttcatcatcagtttctgtgcattaaacatgaatgtttccttcaagatgaagtttttagcccattttgctttctcagaatttaatcaaactgaatgaagtatgtacttacaattaatatttggggatgttcatatttgttccccccttatatgacagttgggatattgtgtggtgctcatctccaggcccctccctgtgttccaggagacagggtcactgtcaccagcagagccagtcagggaaataacagtgtctagcctagctccttgtgaaataagggctgacactgagctcctgctggctcccactgctccctggacacgatgcctgcctgggtcaagggggtgagtctggacagacgtcactctggccatcagtagcctaactaccttcatgacctcccactgtgaacagagtcccggaagctgctggagccatgaaagttggacagagaaatcccacatcactgcagtcagaggggggctatgaaaagaccgtgggggggtttcatactatgaccacccagcactgagccatggctgccactctgtctgatgggagcccccaggggaagatccactcacactgtgctctagaggtcgtttttgtcaccatctttgttctagctggtttgggccagtttctgtagtgcatcctgttttgtccagatcctgttctggtcagcgttgtcatgaccagtgttgtgatcagtgctcagaatacaagccctgatgatctcctaccttatactcactgccttctgtgaaccagatgttctgataagggtcctgttggatcctactcgaatcagttgccacacagaccctcactgagggctgagggccacagacatctgaagataaacagaggtccagagaatgatagcctgtgactgtcctctgtaaagacagctgctccccagatggctgagggctgtctttggctgtgtccttctttctgaatgataatgagggatagagcaggtctctaagcaaaacctcagcaaggccttcctatgtgtgctgctctaacctggacataggtggcactggatacacttagggaagtgaggaaatttatagtcacaaggaagagagagaaaggaggagagagagagaagagcctgtgatgtgtgtatagtaccaacactgatagtatgttctttaatggtttattgttgagtatgatgctcaaatgctcaggttctattccatgcagccgatacatataccttatataggaaaagggcctttgcatatataaattaaggattatgaattgggcagattatcctgtattgaccaggtgggccctaaatgggcctgtctttattaaatgtctgtctatctctcttataaggacatcacgattgtccttataagatagacatagacagacattttaacaaagtctgcagaagagaaggcaatttgaagacagtggcagagattgaagtgatgtggactcaagccaaagaaagttgaagccaccaaaagctggaagaagccaaaaagtagactccccgctggagtccctgcagaagctttgatgacagcctgctcttgacccctgaaactagtgctggacttatggcctccagaactataggaggagaatatatttctgttgctttaagccaccaaagttttagtaatttgttatagcagcccaagaattctaataaaaatggggcttaggataagtccagcctaaaggtagtatgatgatttgcactctccaccttcatttctctaatgttacacataattggttagaagaagatttttatgatggaaacattgtacaggaagccacccagtatatacaggggcatctgttagttacagaataaatattgacagttcttagttgaaaatgacatctgaggctgggcttggtggctcacgcctataatcccagcactttgggaggccaaggcgggtggatcacaaggtcaggagatggagatcatcctggctaacacaatgaaaaatctgaaaaatacaaaatttttcagaaattctgaaaaatacaaaaaattagccagacatggtggcacgtgcctgtagtccaagctactcgggaggctgagacaggagaatcccttaaactcgggaggtggaggttgcagtgagccgagaccacaccactgcactcaggcctgggcaagagagcaagactctgcctccaaaacaaacaaacaaacaaacaaacaaaaaacccaccaaaccaggactttttgaactcagctctgaaccaagtggacctaatagacatctacagaactctccatcccaaatcaacaaaatatacattcttctcagcagcacatcgcacttattctaaaattgaccacaaatgccttatgtaaatgacgagttgatgggtacagcaaacccatatggcacatgtacacctatgtaacctgcaagttgtgcacatgtaccccagaacttaaagtgtaataatacaaaaaaatgacatgtgactagtagtatcttatctagaatcttcattctaagatactcaaggacgcataaaagggaccctaagtagtcttttcatacatatatatgcacatatatatgtatgaaaagcagtcttttcatcaactagagaaaccctcaggacagcccttaatacccttggtgatacatttcagatgagtaaactgttatcagagcccgtagttgaaactattcaacagagatggtttgcccaaagatatgtggtcagcaattgtcagggctgagcttggaacccaggtctgcataaccttaaatatgttgcttccacatggccacgtttgtttcatatacgattgaatggcctttaaattcaaagaagagacaaagccagaagagtggtgtgaaattctcaacacaagctccctgctacctctacaccttaccgtgattactccaattataaactcaggccctcatgcagttttgtctacaaagcaaaacttcctcaaagtctttacaaatactaaatgtctttctttcagattcgagggcaagagcacatcttgcattgccctgaacactttgcatcttttctaccattctcatctttctgtcccagtccttccttctcaaatgatgtcctgtaaatctgatttctcccccaatatgaaaacaaatgaacaaatattcccctacttttctcatatccagaggatacaagagttaatcacatatccagagagtacgagagttaatcaagggatttatgcaagagtgtttacacataacaaggattctggtgctagccatcttcacagtgaaattttctgtgtgtcttgctaaaattgacactaaaaaatgacaagataaaaatatttggaagaacagagggcaaccatgcccttaaggaggtaaaagacacccctgccccttgtgttagtttcctactcctgctgtaacaagttatcagaatcttactagtttcatacaacacaaatttattatcatacagttctggcagtgagaagtctcactgatttataatcaaggtatccgtagttctatattccttctggaagttccaggggagagaatctgattctcagcttttcatcttcaaagatagccccatgttccgggctgcctggcccctttctccatcactgaagcatccctgtccattgtccctattcctctctgactgttacccccactcctccctattataaagacccttctgatgacgctgtctttcctagataattcagctgtttcctaaattttctgaatatccctatgcatgaaaaaaaaagaattggcaagtattcagactatactttccaagaatgagggtttgtccactgttttaggttggatctttcagggacaatgatgcccatgcaggcagcatatttataatgcacagtaaacactaggaggaaacaaggcagtgagagaggaaagagagcagcgataccgaaaatgtcctcagcgagaagctaccacagaggatgaatggagatcaagcccacgtggaaacatgggaaaatgtctcagtatttttccacctaagaagggagggagatggggtatgtatacacctccctgtcctcactgattgagggctttccgagaggatgctcattccaggtgctgtgataggccatgtgtacaggcagggctgccttctccagcttcagatagagcagtgaggaaaagatatggccatggggggtcagcagaagtacagcaaagggaaaagggaaagggtagcaagagtgacaactatattcaccccccccacacacacacacacacacacgaaattgtgtattgcaatccagaactgcttctctctgaacctaaatcttagcaagcagtttaccagtaactgcccttgaaattcaggcccctggaaaggagcagggggttgtgtacaggctataccacagcagtctgcccacccttagtgatgcatgagtaatgctccctggactccccaggttctagtcttctcatgtcgatgtagttgattccacttcccttgctgcacaaccaggctgggatgcctgggcagaggcagacatgtgaggtataggggttcaaatctgtttccaagttttatccagcttcaaagcatttctccgtgtacatgagcggtggcttgacaggagatggagactctctttcctggatgtgaggcaaggaggcaggcgtctgagtcaggatgatgtccctactcactgctaaagagaaaagtggctttgatggtgcagggcagggaaatgcactgagtggtcgccaccctcacagaagagaaagtgttcactgacctggcctttccccagggcctctccctcccattgctttccagaaagccatgatttttgagagccacacctgaacactcacaaacattatggtgggaaaagcagatcagagcattaggcaagttgcattaccttggccttcttcctttggagacaattgatgtggggttctagattgacccagagtttcaagtttatcctgattcaggcttcaacagctggaggaagaaacagagatgttttttgaagtaaacagatctagcattactaatcaacccttcatactgatgacctatgggaaataatacccaagggcagaaaaatgggcagaataaggggagccccaaaccaagacgaagctgctgcccattgagaccctgggtattacagagacctatagctctggataatggaagatctatgagtggcacaggcgctgaggaatcacagcatcattatcgtgcatctgcagggaattgcttgtaaatatactggtaattacaaatgtttaaggtcactacaaatactttggagtgtattaaatatgcttctgataaagactgtttttctcacatgaaacaatgggaaccatgtgacaatcacagaggtgttgttactatagcaaaagggattgttactctccacatccctttaagtaacttgaaggcctgatagacccaccctctaagacttcattagacattccctacgaatggttatactctcctgtatactcccaatacaactctaaaatatattattccatatagtccttaggtttgtattaaagtttgacttttttccttcaaaatatctcttgtcacaacagcggctctagagagaaatacattccctccaggcaaatctatgctgcgctggtctgacctgggaccctggggacattgcccctgtgctgagttactaagatgagccagccctgcagctgtgctcagcctgccccatgccctgctgattgatttgcatgttccagagcacagccccctgccctgaagacttttttatgggctggtcgcaccctgtgcaggagtcagtctcagtcaggacacagcatggacatgagggtccccgctcagctcctggggctcctgctactctggctccgaggtaaggatggagaacactaggaatttactcagccagtgtgctcagtactgactggaacttcagggaagttctctgataacatgattaatagtaagaatatttgtttttatgtttccaatctcaggtgccagatgcgacatccagatgacgcagagcccgtcctcgttgagcgcctccgtcggcgaccgcgtgactatcacttgtcgggcgtcccagtctattagctcctacctgaactggtatcagcagaagccaggaaaggctcccaagctccttatctacgccgcatcatcgctgcaatccggagtgccgtcccggttttcgggctcgggttccggcaccgacttcaccctgaccattagctccctgcaacccgaagatttcgccacctactactgccagcagtcctactcaactccttacaccttcggacaagggaccaaactcgagatcaagcgtaagtacttttttccactgattcttcactgttgctaattagtttactttgtgttcctttgtgtggattttcattagtcggatgccagggatctaacaaacttcattcccaggttaggtacagaggaggggaaattgttccacaggacgctagcttgtggctaatttttaagatttctaaatcaaaataacttcattgggggaaagaggcttgctgagctttcagggacacagtgttacaagtcataacataaaccaaaacaccctgacccctctgcctggcatagaccttcagacacagagcccctgaacaagggcaccccaacacctcatcatatactgaggtcaggggctccccaggtggacaccaggactctgaccccctgcccctcatccaccccgcaggttctcaaccctcaccccccaccacgggagactagagctgcaggatcccaggggaggggtctctcctcccaccccaaggcatcaagcccttctccctgcactcaataaaccctcaataaatattctcattgtcaatcagaaatcttgttttatctcattttttcttttctcacatataattcctagcctttcctgggttctcaatttgtggtggggtttatgttatgacttgtaacactgtgtttcgctgttaatatcactaacctgaccgatgcagagaaaatcttgcaattgagatgcctcacttaactggctagcgcttggctgttccttaagatgaactaattttctatcccttactcatctgactttttgaaagaatctggtactctttggaattgacctgagctaatatctcaaacacaaaaacgctccaaatttaaaaccttataagaaaaagcattaggaaagtgcacttacgcttaatctcgaccttggttccacccccaaaggtaaggggagtggagtagtactgttggcagtagtacactgcgaagtcctcggcctgcaggctgctgatcgtcaaagtgaagtcagtgccggagccgctccccgagaaccgatccggcactccggattcgcgggtcgaggcccagtagatgagcagcttaggcggctgtcctggcttttgctgataccaggcgaggtaattcttgttgtttgaggagtacagcacggactgcgagcttttgcagttgatggtagctctctctcccagggacacggccagggagtcgggtgactgggtcatcacaatttcgccgtacgcacctgtagtcagtaatggacaatgattatacataaacttatacacgctgtctcagatataattgaaatatgtcatttaaatttgctttctagtagtattataagtcagcagataattggatccatattggaaatagtcattatttccaactatatcttaattttttttatttctacacagatattactctgaaaagactgtggcactttttaattcctcaccagagatccagagcaacagagaaatgaagacctgggtctgcaacaccatcttgctgcccctgcctgcctgttgtagctcagttcacaatgcaaacggcccgtttataaaatccaaacagctggtcgtggcctggaggggcctatgcaaaaacaaccagtgagtacaaaagcaaatttcatgagcagtgggttgtgaaagtacccaatgtaaatcaagagccaaaaatatcatcacagaatgtgtaattgtatgaatagaaaagacagaagtcaacttagaagctctaaaactacaaactgctgaaagatctaatgactaggacagcctagtaattttcataggggcataaatgtgaaacgccttgtgcatcgtagaagaaagcagaagagaaagcattcccaatttcttaactgccttttacctatattaatcagtaatatactggcttttacctctgttaatcataataaacaaattctcaataaattttatcgatactcttcaatgcctgctcagcaacattttccgaaggcagctcaagatattaaataactcataagggccaacctcctattgcagcattctttgggatttaaccagtttcccaagactcttttcacaatgttaagatgttagaaatagatccaaaactaggtgatatatcccctagtaaaactgtgaggtcaaacttgtctggctaatgcttccatttaaaaatttctctttcttgatccttcattgtatgtacacaataaatcaggggaaaactttaactgagtgaatcaaagtattctcattattataataggagcttcacacacacacaaaaaaatcaattctattactctcagcctcagttcctaaagccaagttaaagtcctgttctaagatcattgttgcatgaccatatgtattccaggtctaatctaaactgtggataaatcccagcaggacattagagatttttgtgagagtaagcatataggattcagggtttatgagctttagatttttcttgtcaaaatgaatgagagttgccatatctaaaaattattcccagataaataaaattcactacctagaattaatttatgcatataagtagaaatgctatctccctttttaccatccaaagtggaaagcctcatggaactagaaattaatattagaaaaatcagttaataaaagtatgtcatttcatcaattcaataagttataatagcaaaaaaccataataaattatcacttaaatgtcaatacatttataaactatggtacataaataggatattgaatagccattgatgctcctgatgaaaattagcaggcagtgataaatgataaatatgaagcacatgtcaataaataaaataagttttatgtaatttaggagaaaatggtgataatgacacaaaatgtgaattatggatgcatctataaaattctttgtacatttgtgaattgtaaatatttatcttagagacattattactttgtatatgttccatttgctcacctatatgtcccagtctccttacaaatgctatggccaaagaaataggcatacatacatcctttgcaggctgaggcaggaaaaagatcttacggaattttccagtctatcctttatctgtataagcaacttaagaggccatgtgctccaaatggtgcaaatacaagggtttttgttcagcaagacaatggagagctctcactgtgagtaatttttcactattgtcttctgaaatttgggtctgatggccagtattgacttttagaggcttaaataggagtttggtaaagattggtaaatgagggcatttaagatttgccatgggttgcaaaagttaaactcagcttcaaaaatggatttggagaaaaaaagattaaattgctctaaactgaatgacacaaagtaaaaaaaaaaagtgtaactaaaaaggaacccttgtatttctaaggagcaaaagtaaatttatttttgttcactcttgccaaatattgtattggttgttgctgattatgcatgatacagaaaagtggaaaaatacattttttagtctttctcccttttgtttgataaattattttgtcagacaacaataaaaatcaatagcacgccctaagaaaaatcagggaaaagtgaagtgtacctatttgctatgtagaagaggcagcttacttgaaaatcagcagcaatgttgtttttagagtctgtaataagtaataaactcaaaaagacacattctataggaataagggcttcacagatagagctcattttttaaaaatccaatttgtacattagactaaacgtgaaattatctcttattgtaatggtggaaaggtggttattcccaaaagctcaatctcaaagaaatgtgtttaaatgaaaaaaagtaaataattgcattttttaatgaccgtgggtctgtgaaaaaaataggaaatattttaaagagtatgttctttcattatcctctgttattacttgtctacatttttattctgccaagaaggccgtggcaccgcgagctgtagacagagccgcggtctttctcgattgagtggctttggtggccatgccaccgcgctcttggggcagccgccttgccgctagtggccgtggccaccctgtgtctgcccgattgatgctgccgtagccagctttcctgatgcacagtgatacaaataatgccactaagggaaagagaacagaaacgtaatgggcgctggcggccgcataacttcgtatagcatacattatacgaagttatggcgcgccattcgcgactcgagcgcgccgcacacaaaaaccaacacacagatcatgaaaataaagctcttttattggtaccgaattcgccagggagctctcagacgtcgcttggtcggtctttattcgaaccccagagtcccgctcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtttagttcctcaccttgtcgtattatactatgccgatatactatgccgatgattaattgtcaacacgtgctgctgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtggaaggtggcgcgaaggggccaccaaagaacggagccggttggcgcctaccggtggatgtggaatgtgtgcgagccagaggccacttgtgtagcgccaagtgcccagcggggctgctaaagcgcatgctccagactgccttgggaaaagcgcctcccctacccggtagcacgactcgtacgtacgtacgaagttcctattctctagaaagtataggaacttccactgtggcaggtcagcacccctgtttatgttcctgtcacctgccacagccttgactctcataaccaacaggaaaatgaggaggttctagggccctgtgagtaaaaaactgggatgatagggaaaggagaatggaatctcatctgaatcctccttccttgcctacatttgtttaaatttattgagcaaaagggccagactactgattatttctggcaaaacatgttgagtacattttagggtttaacagttttgggtacctttcaaagaaaatatttggttatatgtaaaattggtattttcccactttttaaattcctacttctcctgtttgccattcttctccactccatgagacagtagagacaggattattcactaattctcctctggcggagctggctgaggacaatcagtaagatcttggttgtgagtgtcaaatagattttgtaatttcatagcagacgcaagttcctaatactaaaactctttgattacaattacctcttgctgatagaaaaagggagttcttgaaattttgaaagttggttttaaaaataaaatgcatacactggaagatgcagtatactaaagatgtagtattttccagggatcactgagaaaatacaggatgaggtaaacagttttattttccaaagttcagaatttgagattgggcagactgcaggaatgggagcatgaagagaacataagagagatcagctattgttcaattacactgcccttgattatggtgggtggggatgtggctggtggtggtgatggcagttgatgatgtggatccaaaaaggggccaaatgtgttacttgtgaagaaccacagagttgaaagcactgctgcatggcttcctgggtggagccaagtcttcgcactttccaaactttctctctcctttattactcacatgaaactgccctctctagtattatggtggaaaaagcattctgcaccagctattttcatgggagtatggccaaggataattatttttactacttatggatttttaaaatccaggacagatataaatcctaataccaaaatagtttgatttgcctcagttgccttttgctgactgaaaatagagttcttccaattccaaaagtgggctttgaaaatagacaaaataattcagatggagaacataaagtttacataatgtaccacaggaacaatgcagaattatatgagatttttatttccttctcaaattcttagaattttaaaaatattttactgacatagcattttaaagagaaaatttttagtgctatgttgtcataagaaaatgattcaaagaatgaataaatgcattatttttatatgatactattcctgaaaataaatctgaatcctctattttagttgttaatgcatagaaaaattatgctcttaatatattccattgacaatagtgctctaaatttatatgcttccttaatttgagggctacagtctgataaatatctatctacattttgtcatgcaacttttaaacctaacagaaatgctttcgttaaaaaagcaacagtgctttctctaccataatgctagagagggcagtttcgtgtgagtggtaaaggagaaagtttggaaagtgggaagacagcaattttgcaatatgaccaagtgttatcatgcaaaaaactatctccaaaccagtcatttctaacatgtatttcacttgtgttaccactaatctgcatccatgatctgatcattctgccaaataaatttacacgaacccgagtaatgagtgatgtctgtcacatgtgataacacggtgtagagacacaaaagactgaatcaacagaacaagatcctgatacaggctaagggtttaaataatacttgagatgatttatttcaagataacagattccataggaagtgcaattaaaattccaatgtgtcatttctgttaggtttaaaagttgcgtggcaaaatgtacatagatatttattagaccggagctctcaaactaagaaatatgagacactcaatatgttattgaaaaatactaattaatgcatcattgaccctatattttattacataatgcagtaaaagaaaataaaatgtaagtagcaagagcacgaggtaagcaatgccatcgtcacctttagccctcctgggattgacagcacctagtcaccttgagtttctgcttttctgcgagacaggagataaaatgaaaacccattcaaggtagttagttatattttggagaaagcaaacaaagataccacctacatgatgctgatatcccaaaggcatatattctcaagtcaaaatagtgaaaagtatatgatccccaaaactgaaaggggagagatacaaggagaatcagagcatgatcaaatttaattacaaagaagcctcataatatggtgaactaaatgtaacaaggtttctttgtctgttgccatggcagtgcagaggcaggcaggtggccttggtggtgtaggtggctctgctccgtgaggtcactccggtgggcaggaggcacgaacaccctgagaacacagccttcctccttcctcacagtcattgcctgcccagccatcctcagcagcatgaggtgggacagagtggagagaaagctgttttcttctaaagactaaaaacaaaaacagaaaaaaaaaaaacaaacctggagctttccatcagtgacaaatgtacttttgactcaatcacacatttgagaagttttccagtgagtggatctgcagataaacccacattcgttgtttgtttgttttaatctgaaaatgtgtttacatgattcttgaaaatattttttgactgtaaacttatatttgtagtagcctatttgaagttatcatttactatttcataattgctactaaaaagttattgttaaaaaaatctgtgactctaactgttcttgtttgaaaggaatacgtctttttaagacactcagactccttttaagctcctcatttggccctcctttgtttctgattcaatgtattgtttaatttgttatttatgactaattaatctattaattaattttcacaatcacagaatcaaatgtcctataagttgctatgtcaaagacctgcctgaagatggcacacatgctccacaatgaacaaaggacagcaccatggtctcaggaacactggggaagtaggagtgctggtgtcccattatcaacagggagccctagagttagagtcaaatcttccctgtgacctgggcacctgggaggagccacccgtgtgctgagctgtgggaacctgccccatgccctgagactggaagcacggccttggctgtgtcccacctgctatggactgaattgtgccctcagattcatgttgaaaccctaattttcaatgtgactgtagaaatcagaacctctaaagatgtaattgaggtcatggcagggggtgtccctgatccagtaggattcatgattttgtaggatccagagacctctcctttttcttctctctctctaaccgccttcccctccatggaaaggctgtgtgaagacatggtgagaaggtggcacctacaaaccaggaataaggtctttagcagaaaacaaactctgctgaacgtcgatctgggatattccaggcaccagaaatgtgaaaattaaattctgtggtttagccatccagccccgtgttacggtgtggcagcggaagctgactcatccatcttccccaccctctgtgagcagatcagcttcaggaggccctcgtggacatggggtcccagctttgctcctcttcctcctgtttttccaactctctggtgaagagggaggactcaagcttcatcatcagtttctgtgcattaaacatgaatgtttccttcaagatgaagtttttagcccattttgctttctcagaatttaatcaaactgaatgaagtatgtacttacaattaatatttggggatgttcatatttgttccccccttatatgacagttgggatattgtgtggtgctcatctccaggcccctccctgtgttccaggagacagggtcactgtcaccagcagagccagtcagggaaataacagtgtctagcctagctccttgtgaaataagggctgacactgagctcctgctggctcccactgctccctggacacgatgcctgcctgggtcaagggggtgagtctggacagacgtcactctggccatcagtagcctaactaccttcatgacctcccactgtgaacagagtcccggaagctgctggagccatgaaagttggacagagaaatcccacatcactgcagtcagaggggggctatgaaaagaccgtgggggggtttcatactatgaccacccagcactgagccatggctgccactctgtctgatgggagcccccaggggaagatccactcacactgtgctctagaggtcgtttttgtcaccatctttgttctagctggtttgggccagtttctgtagtgcatcctgttttgtccagatcctgttctggtcagcgttgtcatgaccagtgttgtgatcagtgctcagaatacaagccctgatgatctcctaccttatactcactgccttctgtgaaccagatgttctgataagggtcctgttggatcctactcgaatcagttgccacacagaccctcactgagggctgagggccacagacatctgaagataaacagaggtccagagaatgatagcctgtgactgtcctctgtaaagacagctgctccccagatggctgagggctgtctttggctgtgtccttctttctgaatgataatgagggatagagcaggtctctaagcaaaacctcagcaaggccttcctatgtgtgctgctctaacctggacataggtggcactggatacacttagggaagtgaggaaatttatagtcacaaggaagagagagaaaggaggagagagagagaagagcctgtgatgtgtgtatagtaccaacactgatagtatgttctttaatggtttattgttgagtatgatgctcaaatgctcaggttctattccatgcagccgatacatataccttatataggaaaagggcctttgcatatataaattaaggattatgaattgggcagattatcctgtattgaccaggtgggccctaaatgggcctgtctttattaaatgtctgtctatctctcttataaggacatcacgattgtccttataagatagacatagacagacattttaacaaagtctgcagaagagaaggcaatttgaagacagtggcagagattgaagtgatgtggactcaagccaaagaaagttgaagccaccaaaagctggaagaagccaaaaagtagactccccgctggagtccctgcagaagctttgatgacagcctgctcttgacccctgaaactagtgctggacttatggcctccagaactataggaggagaatatatttctgttgctttaagccaccaaagttttagtaatttgttatagcagcccaagaattctaataaaaatggggcttaggataagtccagcctaaaggtagtatgatgatttgcactctccaccttcatttctctaatgttacacataattggttagaagaagatttttatgatggaaacattgtacaggaagccacccagtatatacaggggcatctgttagttacagaataaatattgacagttcttagttgaaaatgacatctgaggctgggcttggtggctcacgcctataatcccagcactttgggaggccaaggcgggtggatcacaaggtcaggagatggagatcatcctggctaacacaatgaaaaatctgaaaaatacaaaatttttcagaaattctgaaaaatacaaaaaattagccagacatggtggcacgtgcctgtagtccaagctactcgggaggctgagacaggagaatcccttaaactcgggaggtggaggttgcagtgagccgagaccacaccactgcactcaggcctgggcaagagagcaagactctgcctccaaaacaaacaaacaaacaaacaaacaaaaaacccaccaaaccaggactttttgaactcagctctgaaccaagtggacctaatagacatctacagaactctccatcccaaatcaacaaaatatacattcttctcagcagcacatcgcacttattctaaaattgaccacaaatgccttatgtaaatgacgagttgatgggtacagcaaacccatatggcacatgtacacctatgtaacctgcaagttgtgcacatgtaccccagaacttaaagtgtaataatacaaaaaaatgacatgtgactagtagtatcttatctagaatcttcattctaagatactcaaggacgcataaaagggaccctaagtagtcttttcatacatatatatgcacatatatatgtatgaaaagcagtcttttcatcaactagagaaaccctcaggacagcccttaatacccttggtgatacatttcagatgagtaaactgttatcagagcccgtagttgaaactattcaacagagatggtttgcccaaagatatgtggtcagcaattgtcagggctgagcttggaacccaggtctgcataaccttaaatatgttgcttccacatggccacgtttgtttcatatacgattgaatggcctttaaattcaaagaagagacaaagccagaagagtggtgtgaaattctcaacacaagctccctgctacctctacaccttaccgtgattactccaattataaactcaggccctcatgcagttttgtctacaaagcaaaacttcctcaaagtctttacaaatactaaatgtctttctttcagattcgagggcaagagcacatcttgcattgccctgaacactttgcatcttttctaccattctcatctttctgtcccagtccttccttctcaaatgatgtcctgtaaatctgatttctcccccaatatgaaaacaaatgaacaaatattcccctacttttctcatatccagaggatacaagagttaatcacatatccagagagtacgagagttaatcaagggatttatgcaagagtgtttacacataacaaggattctggtgctagccatcttcacagtgaaattttctgtgtgtcttgctaaaattgacactaaaaaatgacaagataaaaatatttggaagaacagagggcaaccatgcccttaaggaggtaaaagacacccctgccccttgtgttagtttcctactcctgctgtaacaagttatcagaatcttactagtttcatacaacacaaatttattatcatacagttctggcagtgagaagtctcactgatttataatcaaggtatccgtagttctatattccttctggaagttccaggggagagaatctgattctcagcttttcatcttcaaagatagccccatgttccgggctgcctggcccctttctccatcactgaagcatccctgtccattgtccctattcctctctgactgttacccccactcctccctattataaagacccttctgatgacgctgtctttcctagataattcagctgtttcctaaattttctgaatatccctatgcatgaaaaaaaaagaattggcaagtattcagactatactttccaagaatgagggtttgtccactgttttaggttggatctttcagggacaatgatgcccatgcaggcagcatatttataatgcacagtaaacactaggaggaaacaaggcagtgagagaggaaagagagcagcgataccgaaaatgtcctcagcgagaagctaccacagaggatgaatggagatcaagcccacgtggaaacatgggaaaatgtctcagtatttttccacctaagaagggagggagatggggtatgtatacacctccctgtcctcactgattgagggctttccgagaggatgctcattccaggtgctgtgataggccatgtgtacaggcagggctgccttctccagcttcagatagagcagtgaggaaaagatatggccatggggggtcagcagaagtacagcaaagggaaaagggaaagggtagcaagagtgacaactatattcaccccccccacacacacacacacacacacgaaattgtgtattgcaatccagaactgcttctctctgaacctaaatcttagcaagcagtttaccagtaactgcccttgaaattcaggcccctggaaaggagcagggggttgtgtacaggctataccacagcagtctgcccacccttagtgatgcatgagtaatgctccctggactccccaggttctagtcttctcatgtcgatgtagttgattccacttcccttgctgcacaaccaggctgggatgcctgggcagaggcagacatgtgaggtataggggttcaaatctgtttccaagttttatccagcttcaaagcatttctccgtgtacatgagcggtggcttgacaggagatggagactctctttcctggatgtgaggcaaggaggcaggcgtctgagtcaggatgatgtccctactcactgctaaagagaaaagtggctttgatggtgcagggcagggaaatgcactgagtggtcgccaccctcacagaagagaaagtgttcactgacctggcctttccccagggcctctccctcccattgctttccagaaagccatgatttttgagagccacacctgaacactcacaaacattatggtgggaaaagcagatcagagcattaggcaagttgcattaccttggccttcttcctttggagacaattgatgtggggttctagattgacccagagtttcaagtttatcctgattcaggcttcaacagctggaggaagaaacagagatgttttttgaagtaaacagatctagcattactaatcaacccttcatactgatgacctatgggaaataatacccaagggcagaaaaatgggcagaataaggggagccccaaaccaagacgaagctgctgcccattgagaccctgggtattacagagacctatagctctggataatggaagatctatgagtggcacaggcgctgaggaatcacagcatcattatcgtgcatctgcagggaattgcttgtaaatatactggtaattacaaatgtttaaggtcactacaaatactttggagtgtattaaatatgcttctgataaagactgtttttctcacatgaaacaatgggaaccatgtgacaatcacagaggtgttgttactatagcaaaagggattgttactctccacatccctttaagtaacttgaaggcctgatagacccaccctctaagacttcattagacattccctacgaatggttatactctcctgtatactcccaatacaactctaaaatatattattccatatagtccttaggtttgtattaaagtttgacttttttccttcaaaatatctcttgtcacaacagcggctctagagagaaatacattccctccaggcaaatctatgctgcgctggtctgacctgggaccctggggacattgcccctgtgctgagttactaagatgagccagccctgcagctgtgctcagcctgccccatgccctgctgattgatttgcatgttccagagcacagccccctgccctgaagacttttttatgggctggtcgcaccctgtgcaggagtcagtctcagtcaggacacagcatggacatgagggtccccgctcagctcctggggctcctgctactctggctccgaggtaaggatggagaacactaggaatttactcagccagtgtgctcagtactgactggaacttcagggaagttctctgataacatgattaatagtaagaatatttgtttttatgtttccaatctcaggtgccagatgcgacatccagatgacgcagagcccgtcctcgttgagcgcctccgtcggcgaccgcgtgactatcacttgtcgggcgtcccagtctattagctcctacctgaactggtatcagcagaagccaggaaaggctcccaagctccttatctacgccgcatcatcgctgcaatccggagtgccgtcccggttttcgggctcgggttccggcaccgacttcaccctgaccattagctccctgcaacccgaagatttcgccacctactactgccagcagtcctactcaactccttacaccttcggacaagggaccaaactcgagatcaagcgtaagtacttttttccactgattcttcactgttgctaattagtttactttgtgttcctttgtgtggattttcattagtcggatgccagggatctaacaaacttcattcccaggttaggtacagaggaggggaaattgttccacaggacgctagcttgtggctaatttttaagatttctaaatcaaaataacttcattgggggaaagaggcttgctgagctttcagggacacagtgttacaagtcataacataaaccaaaacaccctgacccctctgcctggcatagaccttcagacacagagcccctgaacaagggcaccccaacacctcatcatatactgaggtcaggggctccccaggtggacaccaggactctgaccccctgcccctcatccaccccgcaggttctcaaccctcaccccccaccacgggagactagagctgcaggatcccaggggaggggtctctcctcccaccccaaggcatcaagcccttctccctgcactcaataaaccctcaataaatattctcattgtcaatcagaaatcttgttttatctcattttttcttttctcacatataattcctagcctttcctgggttctcaatttgtggtggggtttatgttatgacttgtaacactgtgtttcgctgttaatatcactaacctgaccgatgcagagaaaatcttgcaattgagatgcctcacttaactggctagcgcttggctgttccttaagatgaactaattttctatcccttactcatctgactttttgaaagaatctggtactctttggaattgacctgagctaatatctcaaacacaaaaacgctccaaatttaaaaccttataagaaaaagcattaggaaagtgcacttacgcttaatctcgaccttggttccacccccaaaggtaaggggagtggagtagtactgttggcagtagtacactgcgaagtcctcggcctgcaggctgctgatcgtcaaagtgaagtcagtgccggagccgctccccgagaaccgatccggcactccggattcgcgggtcgaggcccagtagatgagcagcttaggcggctgtcctggcttttgctgataccaggcgaggtaattcttgttgtttgaggagtacagcacggactgcgagcttttgcagttgatggtagctctctctcccagggacacggccagggagtcgggtgactgggtcatcacaatttcgccgtacgcacctgtagtcagtaatggacaatgattatacataaacttatacacgctgtctcagatataattgaaatatgtcatttaaatttgctttctagtagtattataagtcagcagataattggatccatattggaaatagtcattatttccaactatatcttaattttttttatttctacacagatattactctgaaaagactgtggcactttttaattcctcaccagagatccagagcaacagagaaatgaagacctgggtctgcaacaccatcttgctgcccctgcctgcctgttgtagctcagttcacaatgcaaacggcccgtttataaaatccaaacagctggtcgtggcctggaggggcctatgcaaaaacaaccagtgagtacaaaagcaaatttcatgagcagtgggttgtgaaagtacccaatgtaaatcaagagccaaaaatatcatcacagaatgtgtaattgtatgaatagaaaagacagaagtcaacttagaagctctaaaactacaaactgctgaaagatctaatgactaggacagcctagtaattttcataggggcataaatgtgaaacgccttgtgcatcgtagaagaaagcagaagagaaagcattcccaatttcttaactgccttttacctatattaatcagtaatatactggcttttacctctgttaatcataataaacaaattctcaataaattttatcgatactcttcaatgcctgctcagcaacattttccgaaggcagctcaagatattaaataactcataagggccaacctcctattgcagcattctttgggatttaaccagtttcccaagactcttttcacaatgttaagatgttagaaatagatccaaaactaggtgatatatcccctagtaaaactgtgaggtcaaacttgtctggctaatgcttccatttaaaaatttctctttcttgatccttcattgtatgtacacaataaatcaggggaaaactttaactgagtgaatcaaagtattctcattattataataggagcttcacacacacacaaaaaaatcaattctattactctcagcctcagttcctaaagccaagttaaagtcctgttctaagatcattgttgcatgaccatatgtattccaggtctaatctaaactgtggataaatcccagcaggacattagagatttttgtgagagtaagcatataggattcagggtttatgagctttagatttttcttgtcaaaatgaatgagagttgccatatctaaaaattattcccagataaataaaattcactacctagaattaatttatgcatataagtagaaatgctatctccctttttaccatccaaagtggaaagcctcatggaactagaaattaatattagaaaaatcagttaataaaagtatgtcatttcatcaattcaataagttataatagcaaaaaaccataataaattatcacttaaatgtcaatacatttataaactatggtacataaataggatattgaatagccattgatgctcctgatgaaaattagcaggcagtgataaatgataaatatgaagcacatgtcaataaataaaataagttttatgtaatttaggagaaaatggtgataatgacacaaaatgtgaattatggatgcatctataaaattctttgtacatttgtgaattgtaaatatttatcttagagacattattactttgtatatgttccatttgctcacctatatgtcccagtctccttacaaatgctatggccaaagaaataggcatacatacatcctttgcaggctgaggcaggaaaaagatcttacggaattttccagtctatcctttatctgtataagcaacttaagaggccatgtgctccaaatggtgcaaatacaagggtttttgttcagcaagacaatggagagctctcactgtgagtaatttttcactattgtcttctgaaatttgggtctgatggccagtattgacttttagaggcttaaataggagtttggtaaagattggtaaatgagggcatttaagatttgccatgggttgcaaaagttaaactcagcttcaaaaatggatttggagaaaaaaagattaaattgctctaaactgaatgacacaaagtaaaaaaaaaaagtgtaactaaaaaggaacccttgtatttctaaggagcaaaagtaaatttatttttgttcactcttgccaaatattgtattggttgttgctgattatgcatgatacagaaaagtggaaaaatacattttttagtctttctcccttttgtttgataaattattttgtcagacaacaataaaaatcaatagcacgccctaagaaaaatcagggaaaagtgaagtgtacctatttgctatgtagaagaggcagcttacttgaaaatcagcagcaatgttgtttttagagtctgtaataagtaataaactcaaaaagacacattctataggaataagggcttcacagatagagctcattttttaaaaatccaatttgtacattagactaaacgtgaaattatctcttattgtaatggtggaaaggtggttattcccaaaagctcaatctcaaagaaatgtgtttaaatgaaaaaaagtaaataattgcattttttaatgaccgtgggtctgtgaaaaaaataggaaatattttaaagagtatgttctttcattatcctctgttattacttgtctacatttttattctgccaagaaggccgtggcaccgcgagctgtagacagagccgcggtctttctcgattgagtggctttggtggccatgccaccgcgctcttggggcagccgccttgccgctagtggccgtggccaccctgtgtctgcccgattgatgctgccgtagccagctttcctgatgcacagtgatacaaataatgccactaagggaaagagaacagaaacgtaatgggcgctgagctgggaaaaccagggagaagactgatttattagagatttcagaaataaaattcacattcattatgatatctcattagtgaaaatttccattaggggattgtaaataatttaaagcttttttttttttcagtgctatttaattatttcaatatcctctcatcaaatgtatttaaataacaaaagctcaaccaaaaagaaagaaatatgtaattctttcagagtaaaaatcacacccatgacctggccactgagggcttgatcaattcactttgaatttggcattaaataccattaaggtatattaactgattttaaaataagatatattcgtgaccatgtttttaactttcaaaaatgtagctgccagtgtgtgattttatttcagttgtacaaaatatctaaacctatagcaatgtgattaataaaaacttaaacatattttccagtaccttaattctgtgataggaaaattttaatctgagtattttaatttcataatctctaaaatagtttaatgatttgtcattgtgttgctgtcgtttaccccagctgatctcaaaagtgatatttaaggagattattttggtctgcaacaacttgataggactattttagggcctttttaaagctctattaaaactaacttacaacgattcaaaactgttttaaactatttcaaaatgattttagagccttttgaaaactcttttaaacactttttaaactctattaaaactaataagataacttgaaataattttcatgtcaaatacattaactgtttaatgtttaaatgccagatgaaaaatgtaaagctatcaagaattcacccagataggagtatcttcatagcatgtttttccctgcttattttccagtgatcacattattttgctaccatggttattttatacaattatctgaaaaaaattagttatgaagattaaaagagaagaaaatattaaacataagagattcagtctttcatgttgaactgcttggttaacagtgaagttagttttaaaaaaaaaaaaaactatttctgttatcagctgacttctccctatctgttgacttctcccagcaaaagattcttattttacattttaactactgctctcccacccaacgggtggaatcccccagagggggatttccaagaggccacctggcagttgctgagggtcagaagtgaagctagccacttcctcttaggcaggtggccaagattacagttgacctctcctggtatggctgaaaattgctgcatatggttacaggccttgaggcctttgggagggcttagagagttgctggaacagtcagaaggtggaggggctgacaccacccaggcgcagaggcagggctcagggcctgctctgcagggaggttttagcccagcccagccaaagtaacccccgggagcctgttatcccagcacagtcctggaagaggcacaggggaaataaaagcggacggaggctttccttgactcagccgctgcctggtcttcttcagacctgttctgaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagaagtgcccccacctgctcctcagttccagcctgaccccctcccatcctttggcctctgaccctttttccacaggggacctacccctattgcggtcctccagctcatctttcacctcacccccctcctcctccttggctttaattatgctaatgttggaggagaatgaataaataaagtgaatctttgcacctgtggtttctctctttcctcatttaataattattatctgttgttttaccaactactcaatttctcttataagggactaaatatgtagtcatcctaaggcgcataaccatttataaaaatcatccttcattctattttaccctatcatcctctgcaagacagtcctccctcaaacccacaagccttctgtcctcacagtcccctgggccatggtaggagagacttgcttccttgttttcccctcctcagcaagccctcatagtcctttttaagggtgacaggtcttacagtcatatatcctttgattcaattccctgagaatcaaccaaagcaaatttttcaaaagaagaaacctgctataaagagaatcattcattgcaacatgatataaaataacaacacaataaaagcaattaaataaacaaacaatagggaaatgtttaagttcatcatggtacttagacttaatggaatgtcatgccttatttacatttttaaacaggtactgagggactcctgtctgccaagggccgtattgagtactttccacaacctaatttaatccacactatactgtgagattaaaaacattcattaaaatgttgcaaaggttctataaagctgagagacaaatatattctataactcagcaatcccacttctagatgactgagtgtccccacccaccaaaaaactatgcaagaatgttcaaagcagctttatttacaaaagccaaaaattggaaatagcccgattgtccaacaatagaatgagttattaaactgtggtatgtttatacattagaatacccaatgaggagaattaacaagctacaactatacctactcacacagatgaatctcataaaaataatgttacataagagaaactcaatgcaaaagatatgttctgtatgttttcatccatataaagttcaaaaccaggtaaaaataaagttagaaatttggatggaaattactcttagctgggggtgggcgagttagtgcctgggagaagacaagaaggggcttctggggtcttggtaatgttctgttcctcgtgtggggttgtgcagttatgatctgtgcactgttctgtatacacattatgcttcaaaataacttcacataaagaacatcttatacccagttaatagatagaagaggaataagtaataggtcaagaccacgcagctggtaagtgggggggcctgggatcaaatagctacctgcctaatcctgccctcttgagccctgaatgagtctgccttccagggctcaaggtgctcaacaaaacaacaggcctgctattttcctggcatctgtgccctgtttggctagctaggagcacacatacatagaaattaaacgcgtataacttcgtataaagtatcctatacgaagttatgcggccgc